(19) United States
(12) Patent Application Publication
(10) Pub. No.: US 2004/0014676 A1
(43) Pub. Date: Jan. 22, 2004

(54) SRC kinase inhibitors useful for treating osteoporosis

(76) Inventors: Ramalinga Dharanipragada, Belle Mead, NJ (US); Fahad Al-Obeidi, Tucson, AZ (US); Gary Flynn, Tucson, AZ (US); Dasha Cabel, Tucson, AZ (US)

Correspondence Address: David L. Berstein, ARIAD Pharmaceuticals, Inc., 26 Landsdowne Street, Cambridge, MA, 02139-4234, US

(21) Appl. No.: 10/191,171

(22) Filed: Jul. 9, 2002

Related U.S. Application Data
(60) Provisional application No. 60/303,853, filed on Jul. 9, 2001.

(51) Int. Cl. A61K 38/05; A61K 31/198; A61K 31/165

(52) U.S. Cl. 514/19; 514/563; 514/616; 562/450; 564/157

(57) ABSTRACT

Amide containing aromatic compounds having an inhibitory effect on Src kinase including enantiomers, stereoisomers and tautomers thereof, as well as pharmaceutically acceptable salts or solvates of said compound, said compound having the general structure shown in Formulae I through XVIII.

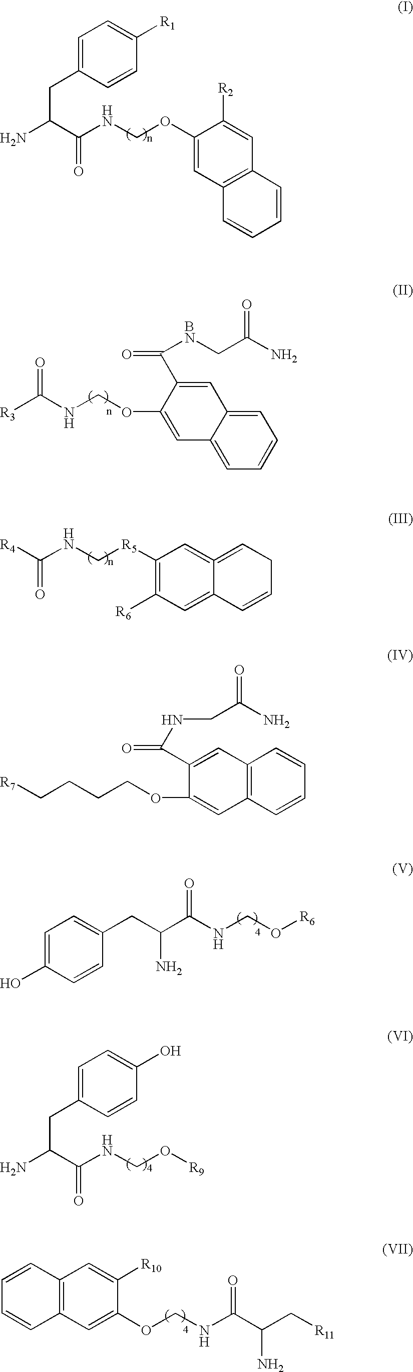

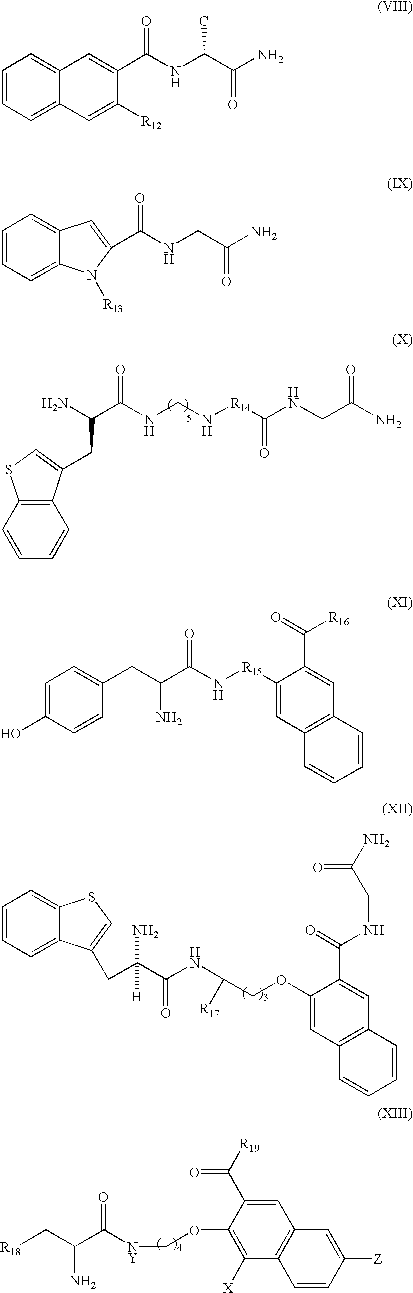

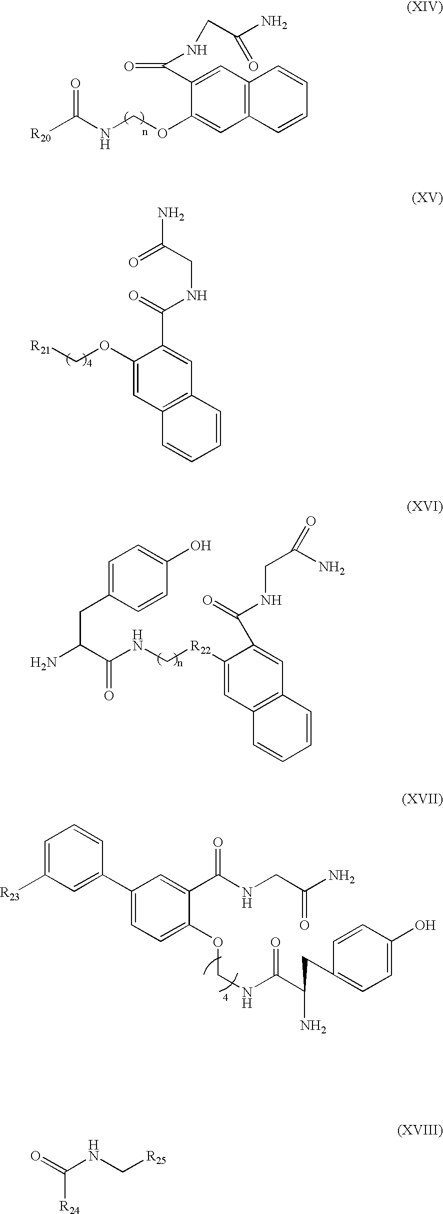

### PRIORITY INFORMATION

This application claims priority to U.S. provisional application No. 60/303,853, filed Jul. 9, 2001, the entire contents of which are hereby incorporated by reference.

### FIELD OF THE INVENTION

The present invention discloses novel compounds containing naphthalene and other aromatic moieties having enzyme inhibiting properties especially for inhibiting protein tyrosine kinases.

### BACKGROUND OF THE INVENTION

The novel compounds of the present invention may have general therapeutic value for the treatment of such diseases as cancer of the bone and osteoporosis. The inventive compounds have been found to inhibit the Src protein tyrosine kinase, a member of the Src family. The Src family consists of the following nine members—Src, Yes, Fgr, Yrk, Fyn, Lyn, Hck, Lck and Blk—which also share the same domain structure. The N-terminal, unique domain contains a myristylation site and frequently a palmitoylation site. It is followed by the regulatory SH3 and SH2 domains, a catalytic domain that is bilobal and has its active site wedged between the two lobes, and a C-terminal regulatory tail that contains the hallmark regulatory tyrosine residue (Tyr527 in Src). Kinase activity is reduced when the latter is phosphorylated and bound to the SH2 domain. The SH2 and SH3 domains bind phosphotyrosyl and proline rich peptides, respectively: through these interactions, they participate in intra and intermolecular regulation of kinase activity, as well as localization and substrate recognition.

There is a wealth of evidence that tyrosine phosphorylation plays a crucial role in many cell regulatory processes. Relevant information is compiled in Fahad Al-Obeidi et al., “Biopolymers” (Peptide Science), 47, 157-223 (1998). Researchers have found that functional perturbation of the kinases results in many illnesses. Thus, there has been a great deal of effort applied in attempts to develop potent and selective inhibitors for these enzymes.

The Src protein kinase plays a role in osteoporosis and other bone diseases. Osteoporosis is defined as a systemic skeletal disease which is characterized by low bone mass and microarchitectural deterioration of bone tissue resulting in an increase in bone fragility and susceptibility to fracture, W. A. Peck, et al., Am. J. Med., 94, 646, (1993) Conference Report. It is estimated that osteoporosis causes 1.5 million fractures annually with a total medical cost of $13.8 billion, National Osteoporosis Foundation, August 1997. The most typical sites of such fractures are the hip, spine, wrist, and ribs. It is also estimated that one out of every two women and one in eight men will have an osteoporosis related fracture in their lifetime. Osteoporosis is most commonly associated with postmenopause and age-related bone tissue loss. In addition, osteoporosis can occur secondarily to various drugs and diseases such as corticosteroids, anticonvulsants, alcohol, malabsorption syndromes, primary biliary cirrhosis, myeloma, thalassemia, thyrotoxicosis, Cushing's syndrome, Turner's syndrome, and primary hyperparathyroidism. Drugs used in the treatment of osteoporosis are generally classified as antiresorptive or formation stimulating. In normal bone tissue, there is a balance between bone formation by osteoblasts and bone resorption by osteoclasts. When the balance of this ongoing process is upset, bone resorption can exceed bone formation resulting in quantitative bone loss. Most of the treatments have involved those that act through inhibition of bone resorption, such as calcium supplements, estrogen, calcitonin, and vitamin D, L. Riggs, West. J. Med., 154, 63 (1991).

Examples of treatments which act through stimulation of bone formation are sodium fluoride, low intermittent dosage of parathyroid hormone, M. Missbach, et al., Rech. Chimie Med., July, 1997, London.

Several reports have disclosed compelling evidence that the protein tyrosine kinase (PTK)p60c-Src (sometimes referred to as c-Src) plays a critical role in osteoclastic function, M. Missbach, et al., ibid. It was reported that, in vitro, kinase inhibitors of c-Src are capable of reducing osteoclastic bone resorption, Ibid. Osteoclasts are bone marrow cells that are responsible for breaking down or remodeling bone. Once an osteoclast comes into contact with the bone surface, it adheres tightly to the bone, flattens out, and begins the process of secreting materials which results in dissolution of the bone. This fundamental action of osteoclasts is dependent on Src kinase. In this case it is clear that at least one of the roles for Src kinase is in the regulation of cytoskeletal changes involved in establishing the close bone cell interface and in polarizing cellular secretion toward the bone surface. Thus, animals genetically engineered to lack Src kinase show abnormalities that indicate a general inability to resorb bone.

In addition, osteoclasts derived from these animals are unable to flatten on bone, nor are they able to dissolve it. Consistent with these results, small molecule inhibitors of Src kinase have been shown to be useful in countering bone loss in animal models of osteoporosis, such as IL-1-induced hypercalcemia, and bone loss in ovariectomized rats. Src kinase inhibitors would be useful for the treatment of disorders marked by inappropriate bone resorption like osteoporosis.

### SUMMARY OF THE INVENTION

The present invention provides aromatic compounds having inhibitory activity against osteoporosis and related tissue loss. The presently disclosed compounds generally contain about one or more aromatic groups, one or more amino groups and one or more amide groups. Usually the compounds also contain an ether moiety. The inventive aromatic compounds include naphthalene carboxamides, hydroxy benzene amides, aminobenzothiophene amines, aminodibenzofuran carboxamides, carboline carboxamides, pyridine carboxamide, benzene carboxamide, and indole carboxamide.

The inventive aromatic compounds have the general structure showing Formulae I through XVIII.

The compound of Formula I has the following structure:

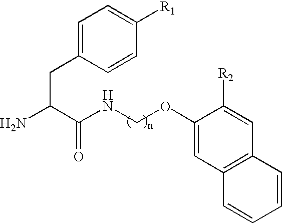

Wherein R₁ is OH or hydrogen,

wherein R₂ is

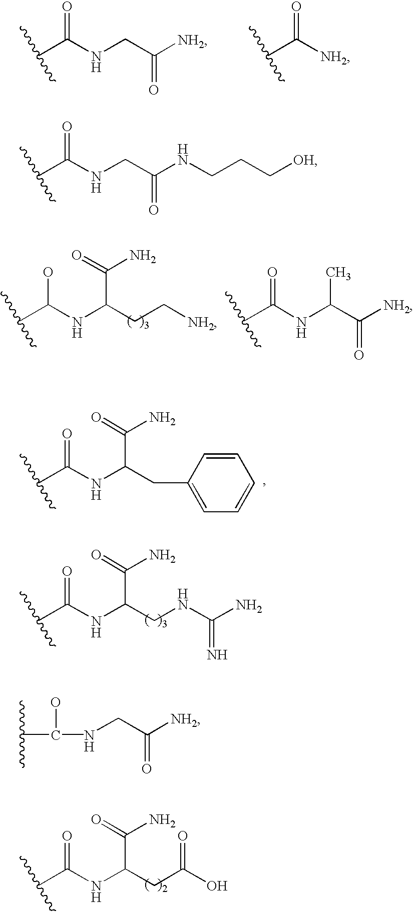

n is an integer having a value of 3 or 4.

The compound of Formula II has the following structure:

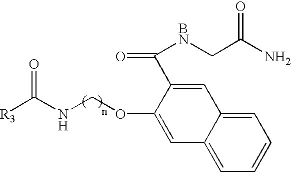

wherein R₃ is

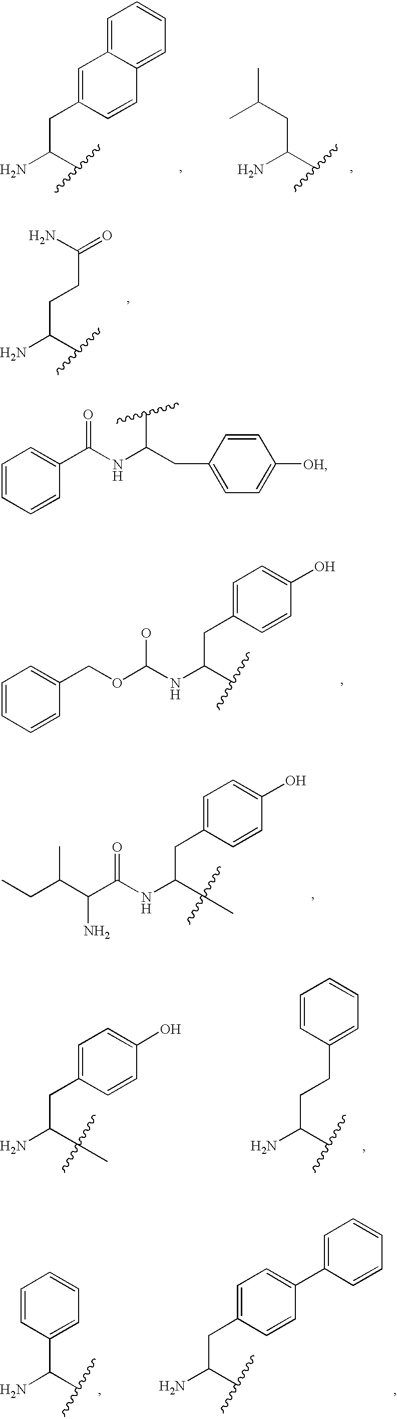

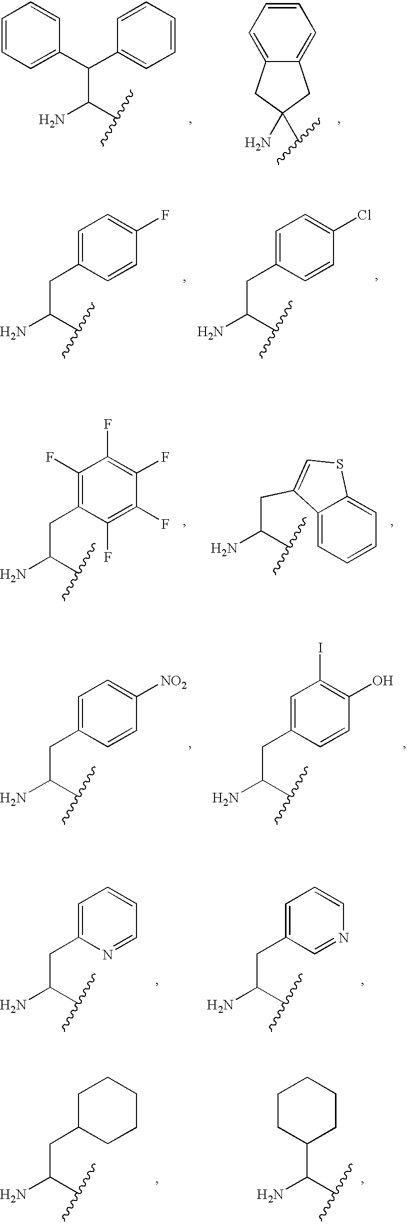

B is —CH₃ or hydrogen;

n is an integer having a value of 3 or 4.

The compound of Formula III has the following structure:

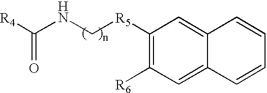

R₄ is

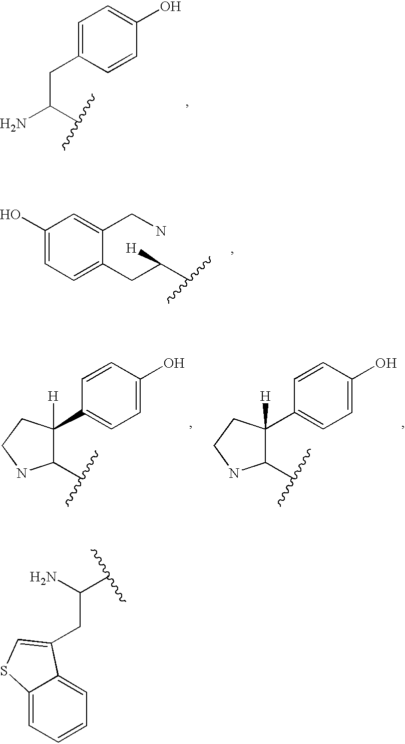

R₅ is oxygen, nitroen,

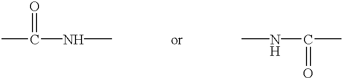

R₆ is

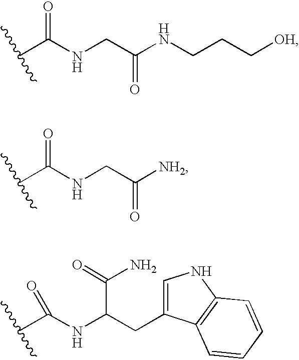

n is an integer having a value of 2 to 6.

The compound of Formula IV has the following structure:

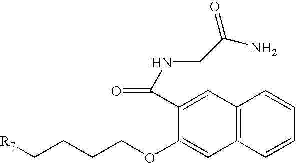

wherein R₇ is

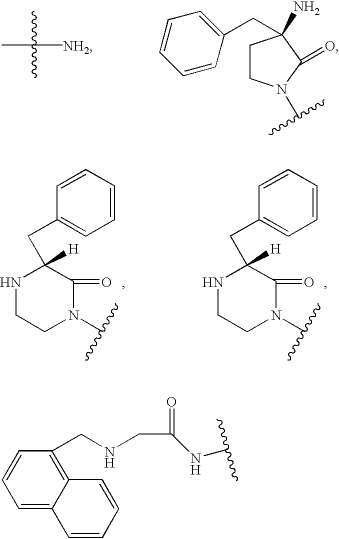

The compound of Formula V has the following structure:

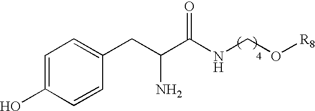

wherein R₈ is

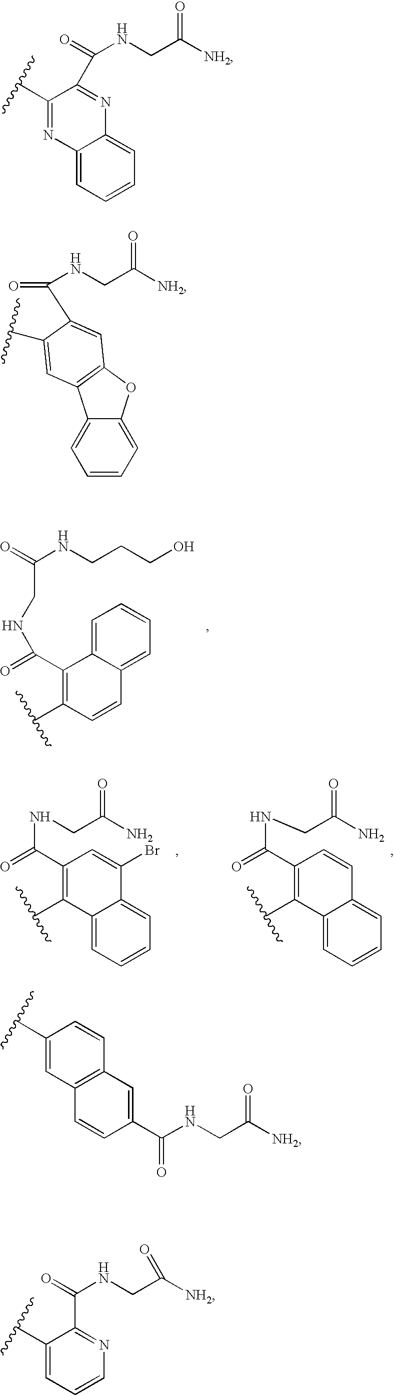

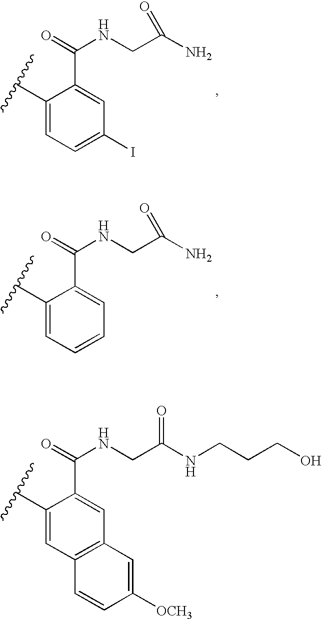

The compound of Formula VI has the following structure:

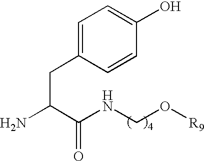

wherein R₉ is

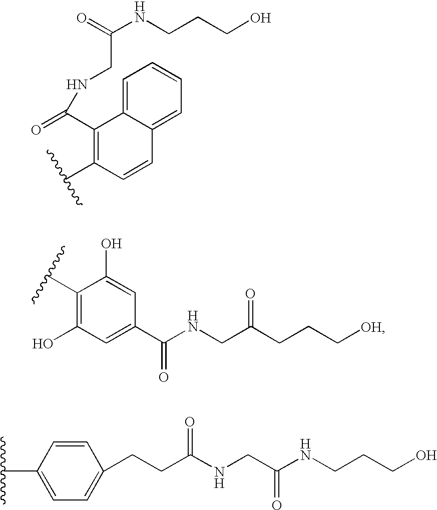

The compound of Formula VII has the following structure:

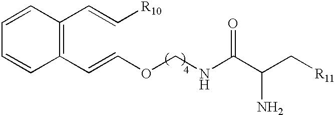

wherein R₁₀ is

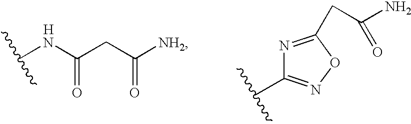

wherein R₁₁ is

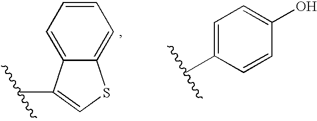

The compound of Formula VIII has the following structure:

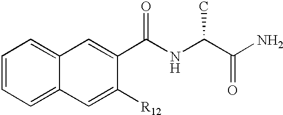

wherein C is hydrogen or methyl, and R₁₂ is

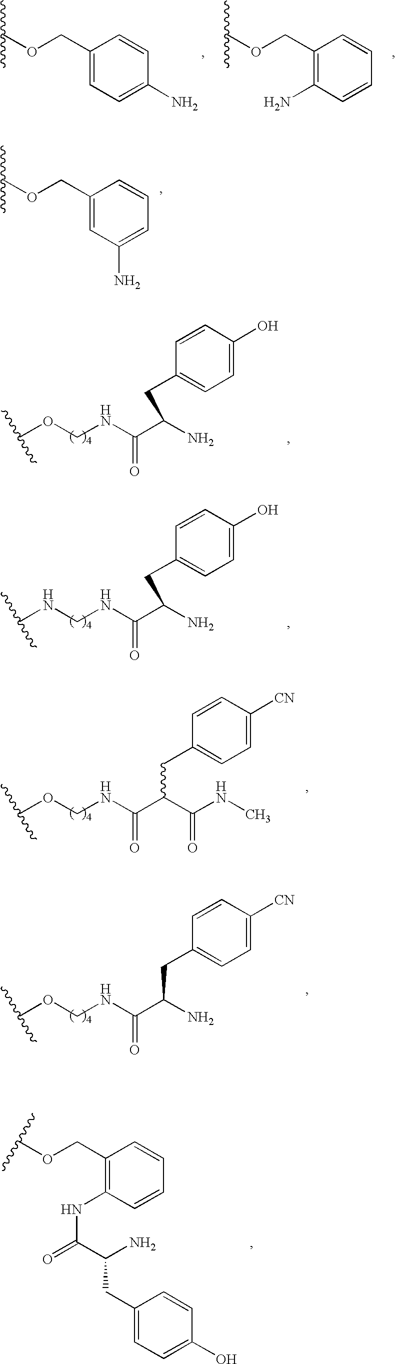

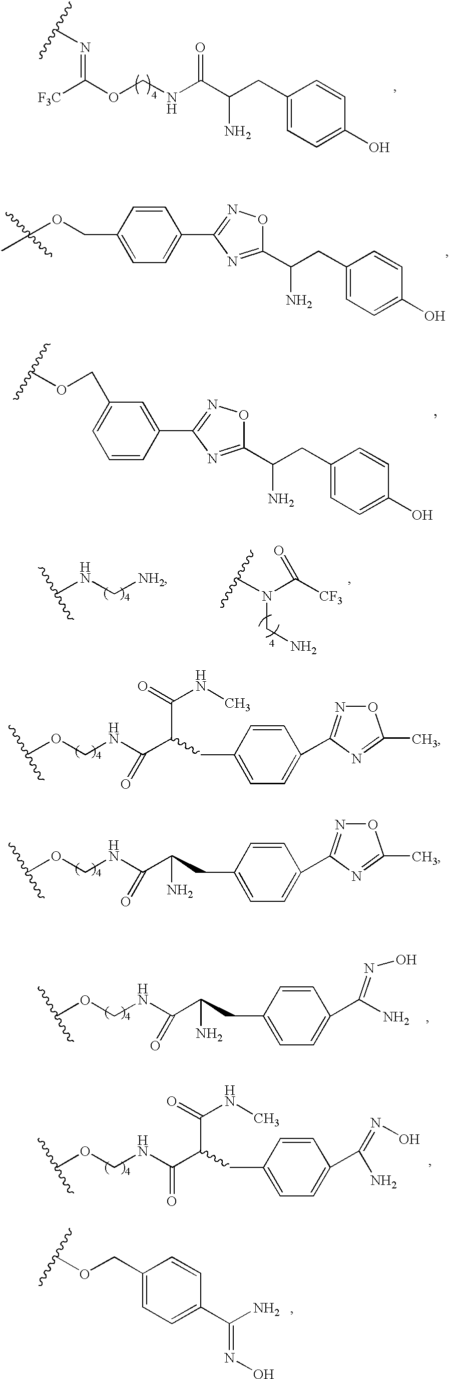

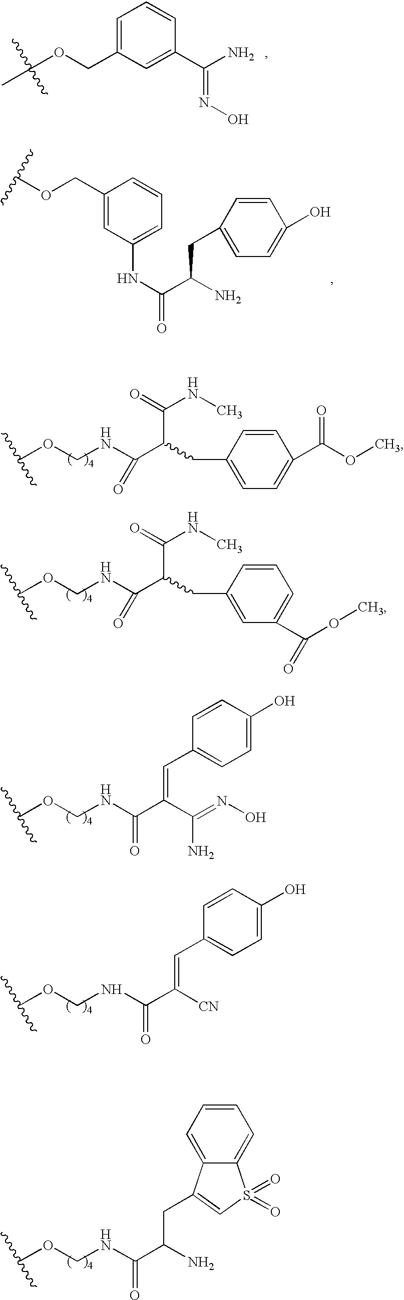

The compound of Formula IX has the following structure:

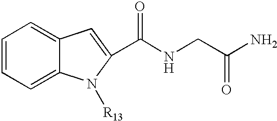

R₁₃ is

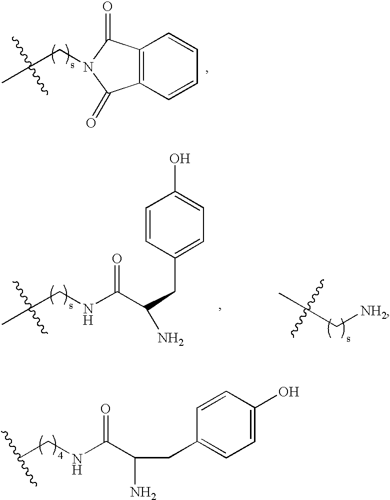

The compound of Formula X has the following structure:

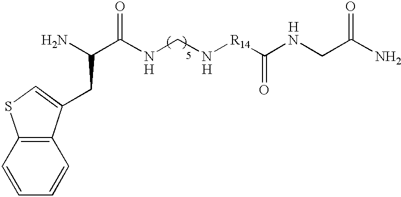

R₁₄ is

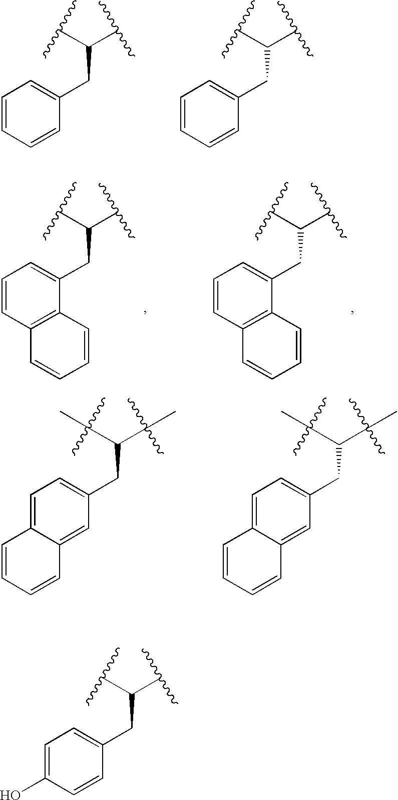

The compound of Formula XI has the following structure:

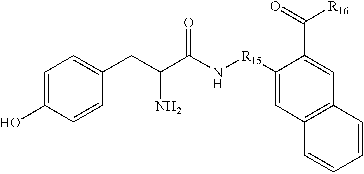

R₁₅ is

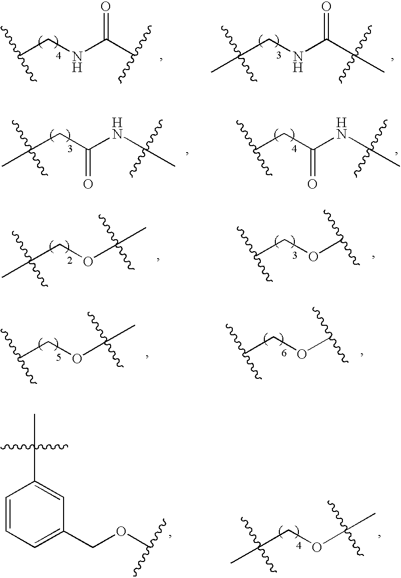

R₁₆ is

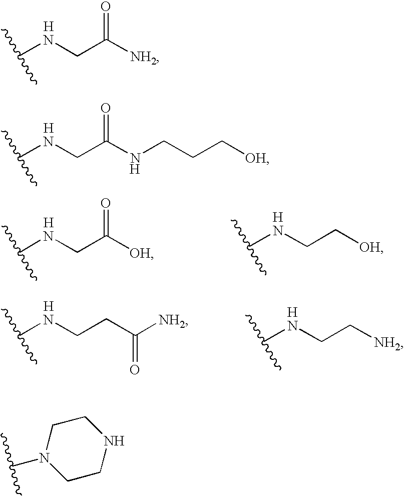

The compound of Formula XII has the following structure:

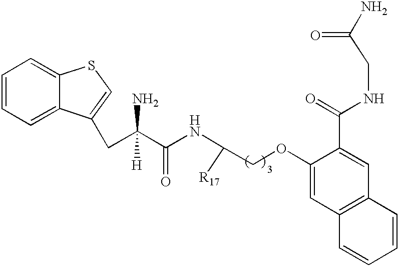

R₁₇ is:

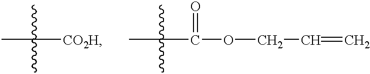

The compound of Formula XIII has the following structure:

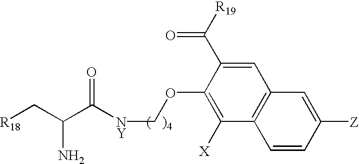

wherein R₁₈ is

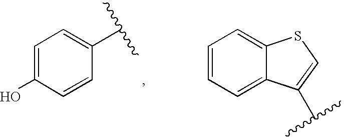

wherein R₁₉ is

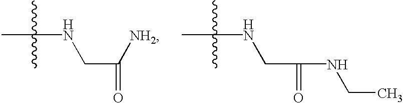

Y is —CH₃ or hydrogen

X is hydrogen or bromine

Z is bromine, —OCH₃, hydrogen.

The compound of Formula XIV has the following structure:

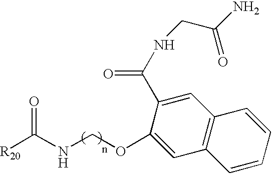

n is an integer having a value of 3 or 4.

wherein R₂₀ is

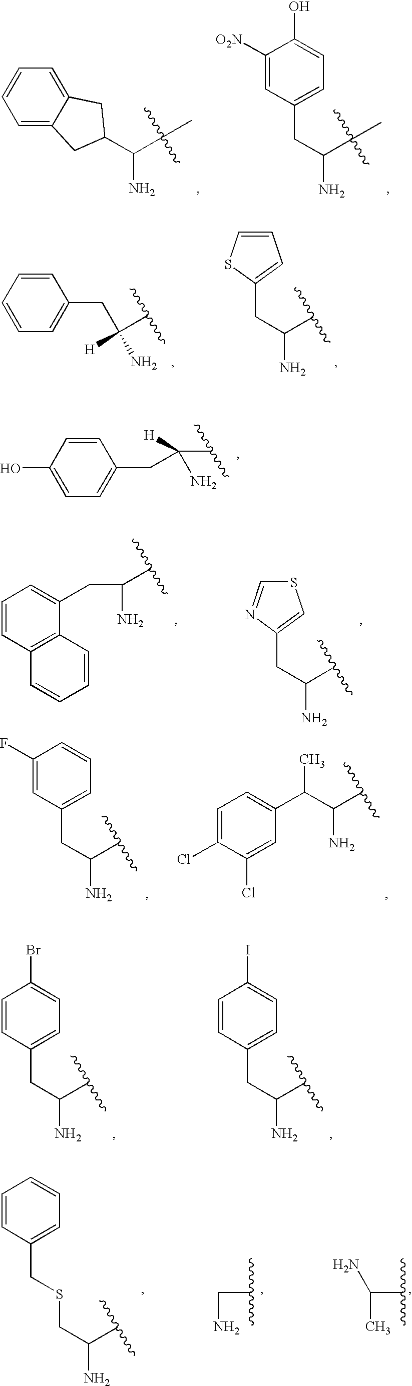

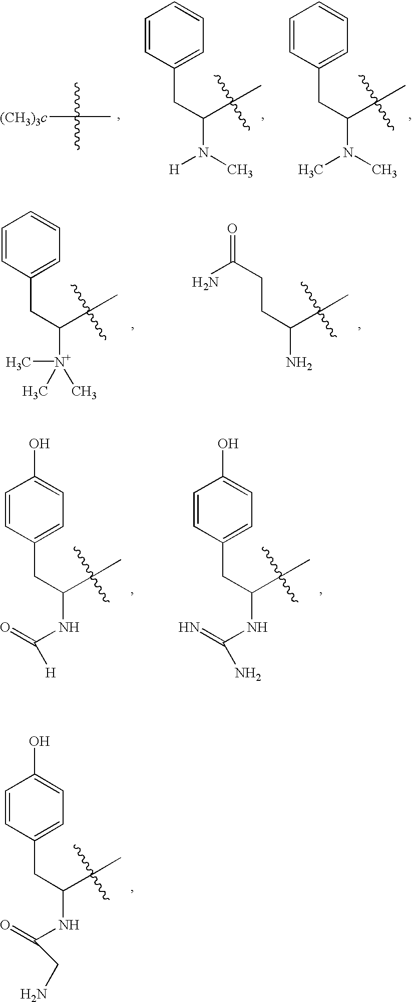

The compound of Formula XV has the following structure:

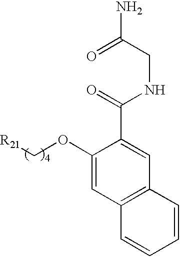

wherein R₂₁ is

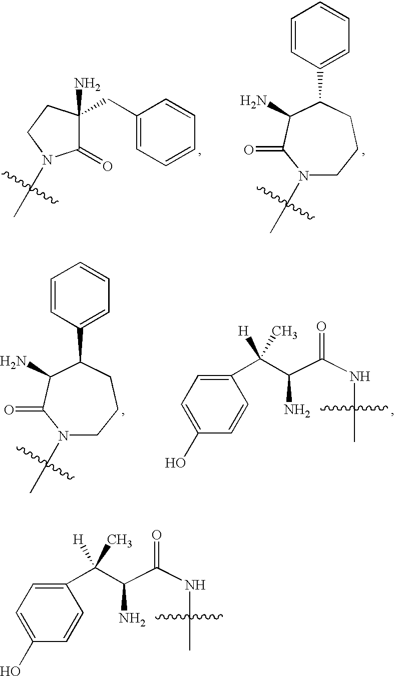

The compound of Formula XVI has the following structure:

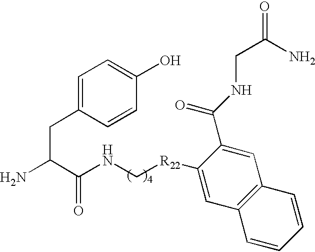

wherein R₂₂ is

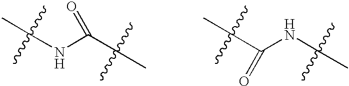

The compound of Formula XVII has the following structure:

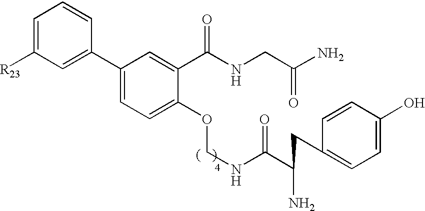

wherein R₂₃ is

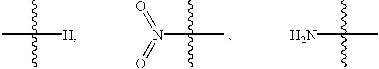

The compound of Formula XVIII has the following structure:

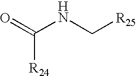

wherein R₂₄

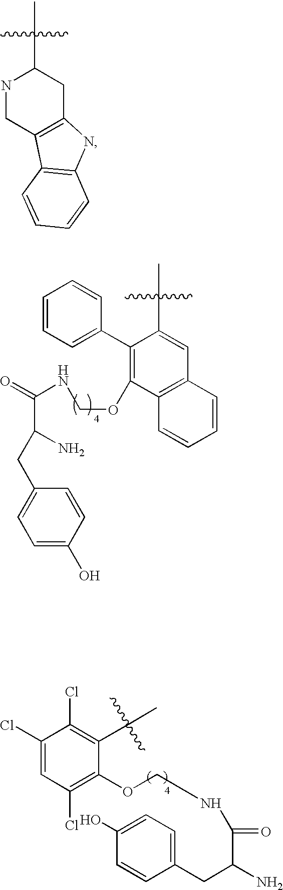

R₂₅ is

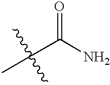

When used herein, unless otherwise defined, the following terms have the given meanings;

alkyl (including the alkyl portions of lower alkoxy)-represents a straight or branched, saturated hydrocarbon chain having from 1 to 10 carbon atoms, preferably from 1 to 6;

aryl—represents a carbocyclic group having from 6 to 14 carbon atoms and having at least one benzenoid ring, with all available substitutable aromatic carbon atoms of the carbocyclic group being intended as possible points of attachment. Preferred aryl groups include 1-naphthyl, 2-naphthyl and indanyl, and phenyl and substituted phenyl;

aralkyl—represents a moiety containing an aryl group linked via a lower alkyl;

alkylaryl—represents a moiety containing a lower alkyl linked via an aryl group;

cycloalkyl—represents a saturated carbocyclic ring having from 3 to 8 carbon atoms, preferably 5 or 6, optionally substituted.

heterocyclic—represents, in addition to the heteroaryl groups defined below, saturated and unsaturated cyclic organic groups having at least one O, S and/or N atom interrupting a carbocyclic ring structure that consists of one ring or two fused rings, wherein each ring is 5-, 6- or 7-membered and may or may not have double bonds that lack delocalized pi electrons, which ring structure has from 2 to 8;

halogen—represents fluorine, chlorine, bromine and iodine;

heteroaryl—represents a cyclic organic group having at least one O, S and/or N atom interrupting a carbocyclic ring structure and having a sufficient number of delocalized pi electrons to provide aromatic character, with the aromatic heterocyclic group having from 2 to 14.

The term “pharmaceutically acceptable salt” is a non-toxic organic or inorganic acid addition salt of the base compounds represented by Formulae I through XVIII.

Included within the scope of the present invention are the individual stereoisomers, diastereomers, tautomers and geometric isomers of Formulae I through XVIII, and enantiomers thereof. The term “stereoisomers” is a general term for all isomers of individual molecules that differ only in the orientation of their atoms in space. It includes geometric (cis/trans) isomers, and isomers of compounds with more than one chiral center that are not mirror images of one another (diastereomers). The term “enantiomer” or “enantiomeric” refers to a molecule that is nonsuperimposable on its mirror image and hence optically active wherein the enantiomer rotates the plane of polarized light in one direction and its mirror image rotates the plane of polarized light in the opposite direction. The term “racemic mixture” or “racemic modification” refers to a mixture of equal parts of enantiomers and which is optically inactive. As used herein the prefixes “(+)” and “(−)” are employed to designate the sign of rotation of the plane of polarized light by the compound, with (+) meaning the compound is dextrorotatory and (−) meaning the compound is levorotatory. For amino acids, the designations L/D, or R/S can be used as described in IUPAC-IUB Joint Commission on Biochemical Nomenclature, Eur. J. Biochem. 138, 9-37 (1984).

A further feature of the invention is pharmaceutical compositions containing as active ingredient a compound of Formulae I through XVIII (or its salt, solvate or isomers) together with a pharmaceutically acceptable carrier or excipient.

The invention also provides methods for administering to a patient suffering from one or more of the aforesaid diseases a therapeutically effective inhibitory amount of a compound of Formulae I through XVIII, or pharmaceutical compositions comprising a compound of Formulae I through XVIII.

### DETAILED DESCRIPTION OF THE INVENTION

The present invention provides novel compounds of Formulae I through XVIII discussed above. The following compounds which fall within Formulae I through XVIII are characterized by structure, IUPAC name and activity using phosphorylation assay for protein tyrosine Src kinase.

| | | | IC50 |
|---|---|---|---|
| Ex. # | Compound Structure | Compound Name | (μM) |
| 1 | 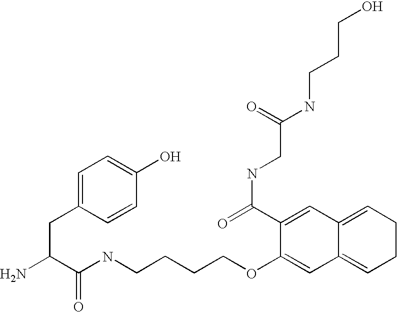 | 3-[4-[[2(S)-AMINO-3-(4-HYDROXY- PHENYL)-1-OXOPROPYL]AMINO]- BUTOXY]-N-[2-[(3-HYDROXYPROPYL)- AMINO]-2-OXOETHYL]-2- NAPHTHALENECARBOXAMIDE | 0.49 |
| 2 | 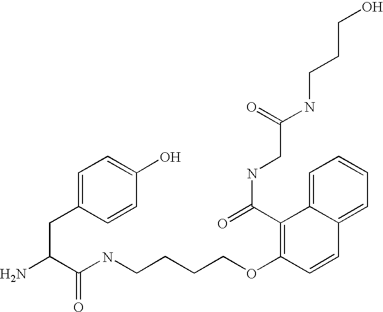 | 3-[4-[[2(S)-AMINO-3-(4-HYDROXY- PHENYL)-1-OXOPROPYL]AMINO]- BUTOXY]-N-[2-[(3-HYDROXYPROPYL)- AMINO]-2-OXOETHYL]-1- NAPHTHALENECARBOXAMIDE | 47 |
| 3 | 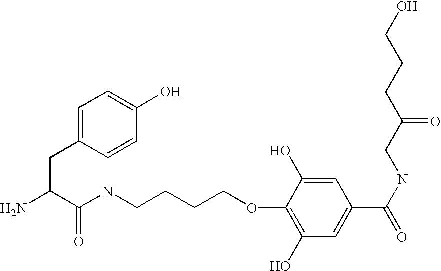 | ALPHA(S)-AMINO-N-[4-[2,6-DI- HYDROXY-4-[[(5-HYDROXY-2-OXO- PENTYL)AMINO]CARBONYL]- PHENOXY] BUTYL]-4- HYDROXYBENZENEPROPANAMIDE | 500 |
| 4 | 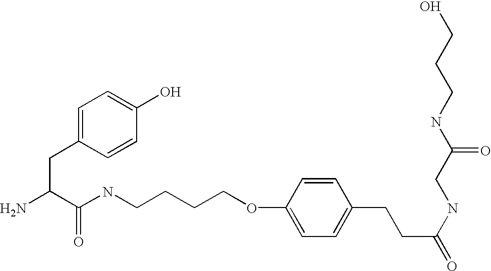 | ALPHA(S)-AMINO-N-[4-[4-[[[(5- HYDROXY-2-OXOPENTYL)AMINO]- CARBONYL]ETHYL]PHENOXY]- BUTYL]-4-HYDROXYBENZENE- PROPANAMIDE | 500 |
| 5 | 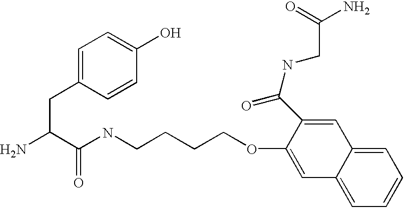 | 3-[4-[[2(S)-AMINO-3-(4-HYDROXY- PHENYL)-1-OXOPROPYL]AMINO]- BUTOXY]-2-[N-ACETAMIDO]- NAPHTHALENECARBOXAMIDE | 0.47 |
| 6 | 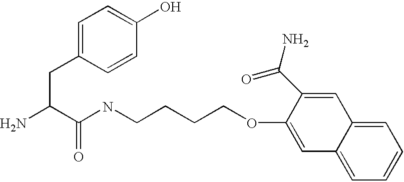 | 3-[4-[[2(S)-AMINO-3-(4-HYDROXY- PHENYL)-1-OXOPROPYL]AMINO]- BUTOXY]-NAPHTHALENE-2- CARBOXAMIDE | 47 |
| 7 | 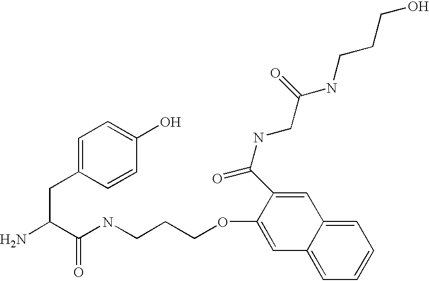 | 3-[3-[[2(S)-AMINO-3-(4-HYDROXY- PHENYL)-1-OXOPROPYL]AMINO]- PROPOXY]-N-[2-[(3-HYDROXY- PROPYL)AMINO]-2-OXOETHYL]-2- NAPHTHALENECARBOXAMIDE | 45 |
| 8 | 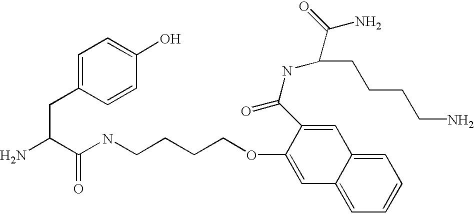 | 3-[4-[[2(S)-AMINO-3-(4-HYDROXY- PHENYL)-1-OXOPROPYL]AMINO]- BUTOXY]-NAPHTHALENE-2-[N-[[(4- AMINOBUTYL)(AMIDO)]METHYL]- ACETAMIDO]CARBOXAMIDE | 348 |
| 9 | 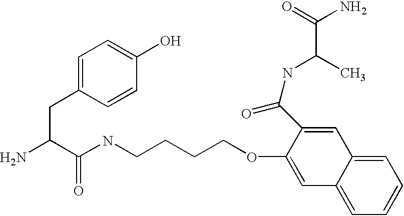 | 3-[4-[[2(S)-AMINO-3-(4-HYDROXY- PHENYL)-1-OXOPROPYL]AMINO]- BUTOXY]-NAPHTHALENE-2-N-[(1- AMIDO)ETHYL]CARBOXAMIDE | 25 |
| 10 | 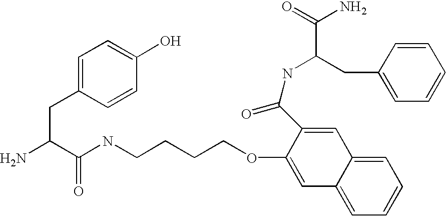 | 3-[4-[[2(S)-AMINO-3-(4-HYDROXY- PHENYL)-1-OXOPROPYL]AMINO]- BUTOXY]-NAPHTHALENE-2-[N- [(AMIDO)(BENZYL)METHYL]]- CARBOXAMIDE | 500 |
| 11 | 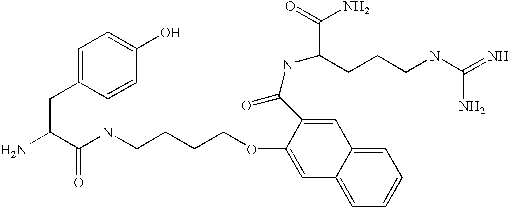 | 3-[4-[[2(S)-AMINO-3-(4-HYDROXY- PHENYL)-1-OXOPROPYL]AMINO]- BUTOXY]-NAPHTHALENE-2-[N- [[AMIDO][(3-AMINOIMINOMETHYL)- PROPYLAMINO]METHYL]]- CARBOXAMIDE | 113 |
| 12 | 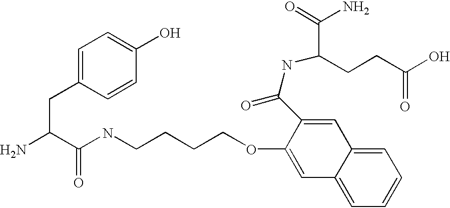 | 3-[4-[[2(S)-AMINO-3-(4-HYDROXY- PHENYL)-1-OXOPROPYL]AMINO]- BUTOXY]-NAPHTHALENE-2-[N- [(AMIDO)(2-CARBOXYEHTYL)- METHYL]]CARBOXAMIDE | 308 |
| 13 | 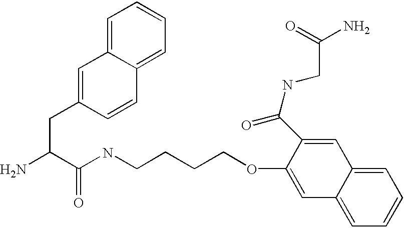 | 3-[4-[[2(S)-AMINO-3-(NAPTHYL-2-YL)-1- OXOPROPYL]AMINO]BUTOXY]- NAPHTHALENE-2-[N-(2- AMIDOMETHYL)]CARBOXAMIDE | 0.45 |
| 14 | 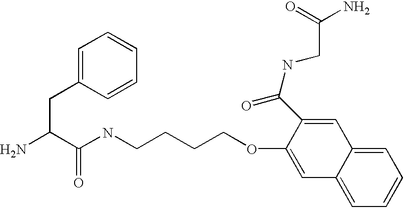 | 3-[4-[[2(S)-AMINO-3-(PHENYL)-1- OXOPROPYL]AMINO]BUTOXY]- NAPHTHALENE-2-[N-(2- AMIDOMETHYL)]CARBOXAMIDE | 0.98 |
| 15 | 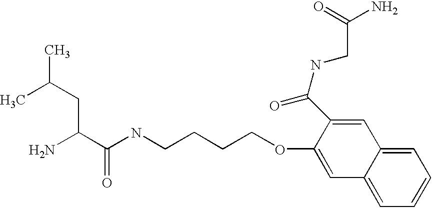 | 3-[4-[[2(S)-AMINO-3-(ISOPROPYL)-1- OXOPROPYL]AMINO]BUTOXY]- NAPHTHALENE-2-[N-(2- AMIDOMETHYL)]CARBOXAMIDE | 4.8 |
| 16 | 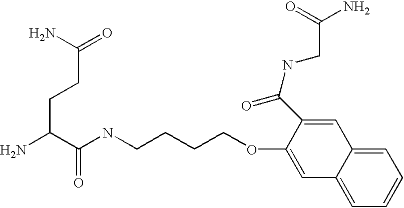 | 3-[4-[[2(S)-AMINO-3-(ACETAMIDO)-1- OXOPROPYL]AMINO]BUTOXY]- NAPHTHALENE-2-[N-(2- AMIDOMETHYL)]CARBOXAMIDE | 16 |
| 17 | 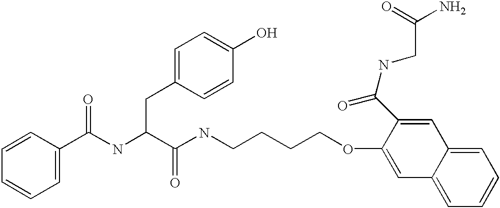 | 3-[4-[[2(S)-(N-BENZOYLAMINO)-3-(4- HYDROXYPHENYL)-1-OXOPROPYL]- AMINO]BUTOXY]-NAPHTHALENE-2- [N-(2-AMIDOMETHYL)]CARBOXAMIDE | 23 |
| 18 | 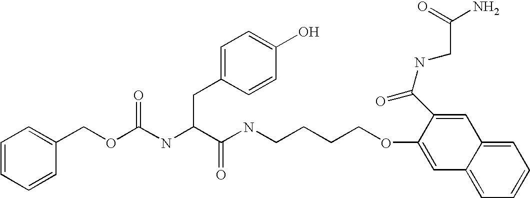 | 3-[4-[[2(S)-(N-BENZYLOXY- CARBONYL)AMINO-3-(4-HYDROXY- PHENYL)-1-OXOPROPYL]AMINO]- BUTOXY]-NAPHTHALENE-2-[N-(2- AMIDOMETHYL)]CARBOXAMIDE | 32 |
| 19 | 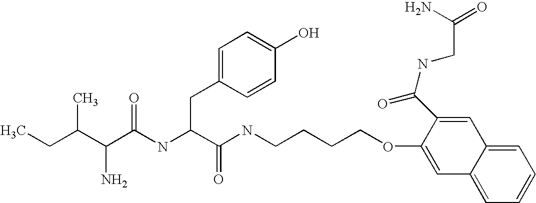 | 2-[4-[[2-(2(S)-AMINO-3-METHYL- PENTANOYL)AMINO-2-(4-HYDROXY- PHENYL)-1-OXOETHYL]AMINO]- BUTOXY]-NAPHTHALENE-2-[N-(2- AMIDOMETHYL)]CARBOXAMIDE | 12 |
| 20 | 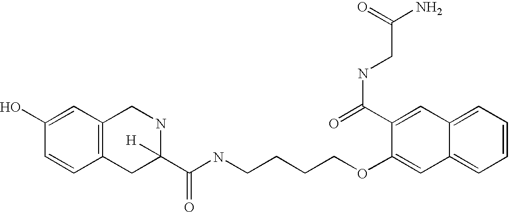 | 3-[N-[[(7-HYDROXY)-1,2,3,4-TETRA- HYDROISOQUINOLIN-3-YL]- CARBONYL]AMINO]BUTOXY]- NAPHTHALENE-2-[N-(2- AMIDOMETHYL)]CARBOXAMIDE | 3.8 |
| 21 | 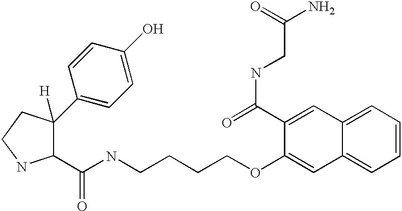 | 3-[N-[[[(3-(R)4-HYDROXYPHENYL)- TETRAHYDROPYRROL-2-YL]- CARBONYL]AMINO]BUTOXY]- NAPHTHALENE-2-[N-(2- AMIDOMETHYL)]CARBOXAMIDE | 4.6 |
| 22 | 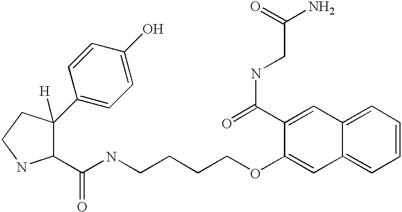 | 3-[N-[[[(3-(S)4-HYDROXYPHENYL)- TETRAHYDROPYRROL-2-YL]- CARBONYL]AMINO]BUTOXY]- NAPHTHALENE-2-[N-(2- AMIDOMETHYL)]CARBOXAMIDE | 11 |
| 23 | 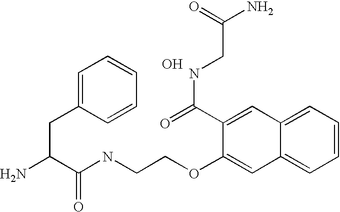 | 3-[4-[[2(S)-AMINO-3-(4-HYDROXY- PHENYL)-1-OXOPROPYL]AMINO]- ETHOXY]-2-[N-ACETAMIDO]- NAPHTHALENECARBOXAMIDE | 3.4 |
| 24 | 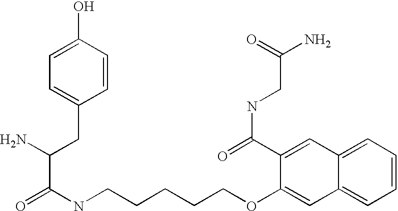 | 3-[4-[[2(S)-AMINO-3-(4-HYDROXY- PHENYL)-1-OXOPROPYL]AMINO]- PENTOXY]-NAPHTHALENE-2-[N- AETAMIDO]CARBOXAMIDE | 6.6 |
| 25 | 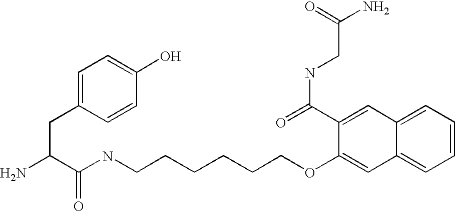 | 3-[4-[[2(S)-AMINO-3-(4-HYDROXY- PHENYL)-1-OXOPROPYL]AMINO]- HEXOXY]-2-[N-ACETAMIDO]- NAPHTHALENECARBOXAMIDE | 7.1 |
| 26 | 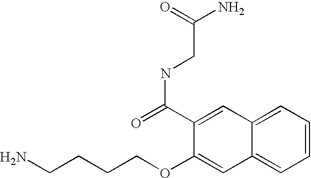 | 3-(4-AMINOBUTOXY)-NAPHTHALENE- 2-[N-ACETAMIDO]CARBOXAMIDE | 25 |
| 27 | 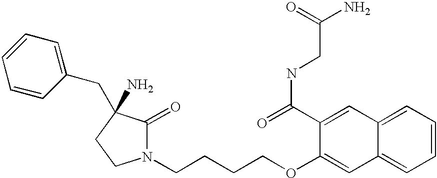 | 3-[4-[(2-OXO-3(R)-AMINO-3-BENZYL)- PYRROLINDIN-1-YL]BUTOXY]- NAPHTHALENE-2-(N-ACETAMIDO)- CARBOXAMIDE | 27 |
| 28 | 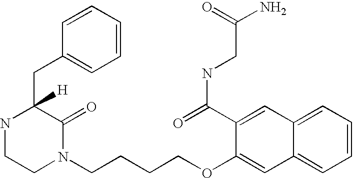 | 3-[4-[(2-OXO-3(S)-BENZYL)-PIPERAZIN- 1-YL]BUTOXY]-NAPHTHALENE-2-(N- ACETAMIDO)CARBOXAMIDE | 27 |
| 29 | 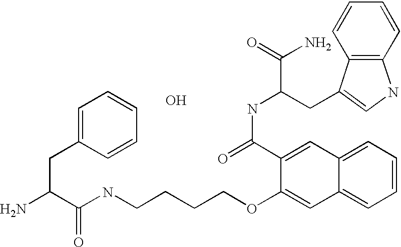 | 3-[[[2(S)-AMINO-3-(4-HYDROXY- PHENYL)-1-OXO]PROPYL]AMINO]- BUTOXY]NAPHTHALENE-2-[N- [(AMIDO)[2-(INDOL-3-YL)METHYL]- METHYL]CARBOXAMIDE | 60 |
| 30 | 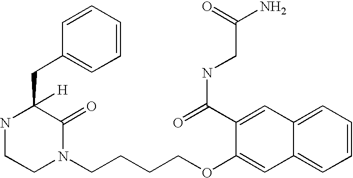 | 3-[4-[(2-OXO-3-(R)-BENZYL)- PIPERAZIN-1-YL]BUTOXY]- NAPHTHALENE-2-(N- ACETAMIDO)CARBOXAMIDE | 8.8 |
| 31 | 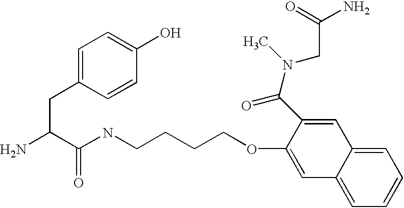 | 3-[[[[2(S)-AMINO-3-(4-HYDROXY- PHENYL)-1-OXO]PROPYL]AMINO]- BUTOXY]NAPHTHALENE-2-[N- ACETAMIDO-N- METHYL]CARBOXAMIDE | 400 |
| 32 | 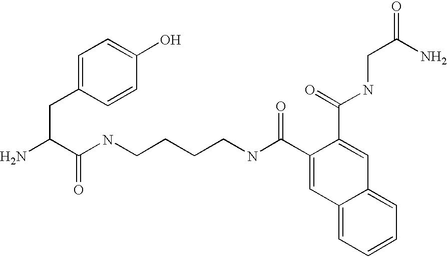 | 3-[[[[[[2(S)-AMINO-3-(4-HYDROXY- PHENYL)-1-OXO]PROPYL]AMINO]- BUTYL]AMINO]CARBONYL]- NAPHTHALENE-2-(N- ACETAMIDO)CARBOXAMIDE | 400 |
| 33 | 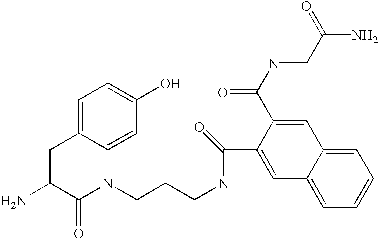 | 3-[[[[[[2(S)-AMINO-3-(4-HYDROXY- PHENYL)-1-OXO]PROPYL]AMINO]- PROPYL]-AMINO]CARBONYL]- NAPHTHALENE-2-(N- ACETAMIDO)CARBOXAMIDE | 400 |
| 34 | 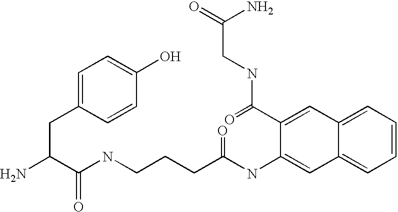 | 3-[[[[[[2(S)-AMINO-3-(4-HYDROXY- PHENYL)-1-OXO]PROPYL]AMINO]- PROPYL]CARBONYL] AMINO]- NAPHTHALENE-2-(N-ACETAMIDO)- CARBOXAMIDE | 252 |
| 35 | 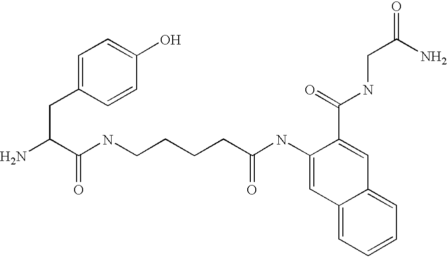 | 3-[[[[[[2(S)-AMINO-3-(4-HYDROXY- PHENYL)-1-OXO]PROPYL]AMINO]- BUTYL]CARBONYL] AMINO]- NAPHTHALENE-2-(N- ACETAMIDO)CARBOXAMIDE | 209 |
| 36 | 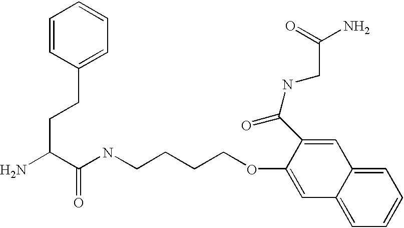 | 3-[[4-[(2(S)-AMINO-3-(BENZYL-1- OXO)PROPYL]AMINO]BUTOXY]- NAPHTHALENE-2-(N- ACETAMIDO)CARBOXAMIDE | 7.9 |
| 37 | 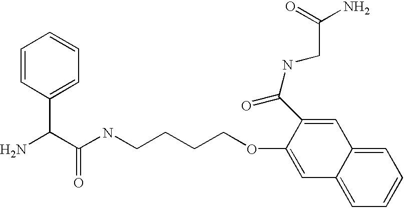 | 3-[[4-[(2(S)-AMINO-2-PHENYL-1- OXO)ETHYL]AMINO]BUTOXY]- NAPHTHALENE-2-(N- ACETAMIDO)CARBOXAMIDE | 3.3 |
| 38 | 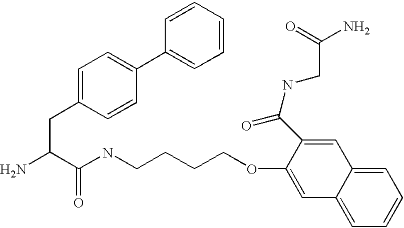 | 3-[[4-[[2(S)-AMINO-3-(BIPHEN-1-YL)-1- OXO]PROPYL]AMINO]BUTOXY]- NAPHTHALENE-2-(N- ACETAMIDO)CARBOXAMIDE | 30 |
| 39 | 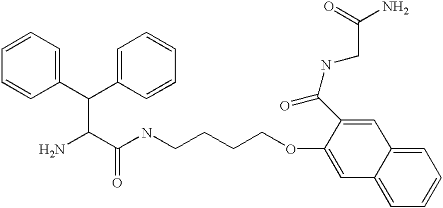 | 3-[[4-[(2(S)-AMINO-3,3-BISPHENYL-1- OXO)PROPYL]AMINO]BUTOXY]- NAPHTHALENE-2-(N-ACETAMIDO)- CARBOXAMIDE | 1.7 |
| 40 | 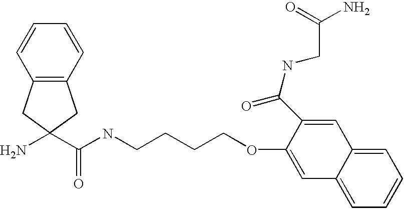 | 3-[4-[n-[1-OXO-2-(2-AMINOINDAN-2- YL)]ETHYL]-AMINOBUTOXY]- NAPHTHALENE-2-(N- ACETAMIDO)CARBOXAMIDE | 12 |
| 41 | 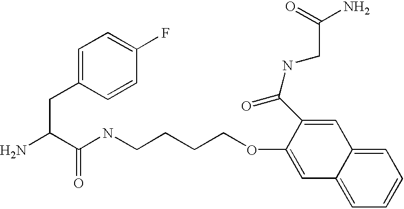 | 3-[[4-[[2(S)-AMINO-3-(4-FLUORO- PHENYL)-1-OXO]PROPYL]AMINO]- BUTOXY]-NAPHTHALENE-2- (N-ACETAMIDO)CARBOXAMIDE | 0.31 |
| 42 | 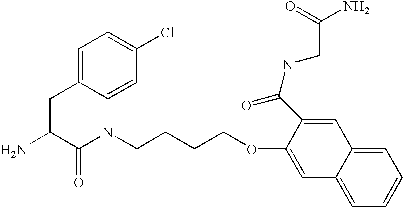 | 3-[[4-[(2(S)-AMINO-3-(4-CHLORO- PHENYL)-1-OXO)PROPYL]AMINO]- BUTOXY]-NAPHTHALENE-2- (N-ACETAMIDO)CARBOXAMIDE | 0.88 |
| 43 | 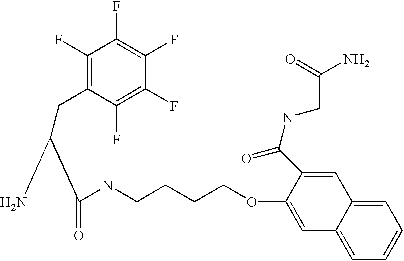 | 3-[[4-[[2(S)-AMINO-3-(2,3,4,5,6- PENTAFLUORO)PHENYL-1-OXO]- PROPYL]AMINO]BUTOXY]- NAPHTHALENE-2- (N-ACETAMIDO)CARBOXAMIDE | 1.38 |
| 44 | 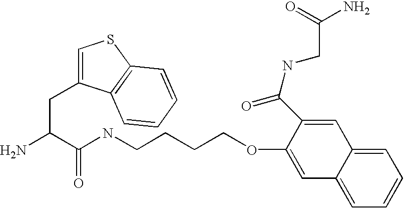 | 3-[[4-[[2(S)-AMINO-3(BENZOTHIOPHEN- 3-YL)-1-OXO]PROPYL]AMINO]- BUTOXY]-NAPHTHALENE-2- (N-ACETAMIDO)CARBOXAMIDE | 0.23 |
| 45 | 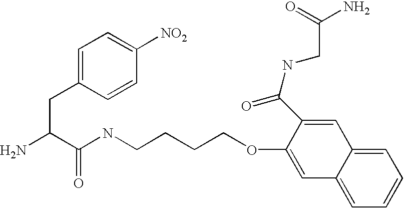 | 3-[[4-[[2(S)-AMINO-3(4-NITROPHENYL)- 1-OXO]PROPYL]AMINO]BUTOXY]- NAPHTHALENE-2-(N-ACETAMIDO)- CARBOXAMIDE | 1.47 |
| 46 | 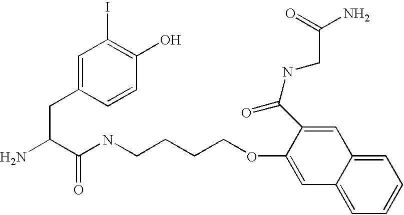 | 3-[[4-[[2(S)-AMINO-3(3-IODO-4- HYDROXYPHENYL)-1-OXO]PROPYL]- AMINO]BUTOXY]-NAPHTHALENE-2- (N-ACETAMIDO)CARBOXAMIDE | 0.31 |
| 47 | 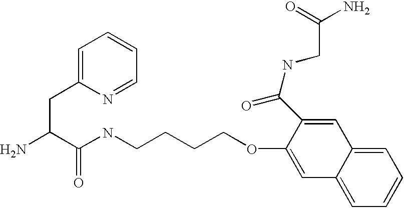 | 3-[[4-[[2(S)-AMINO-3(PYRIDIN-2-YL)-1- OXO]PROPYL]AMINO]BUTOXY]- NAPHTHALENE-2-(N-ACETAMIDO)- CARBOXAMIDE | 17 |
| 48 | 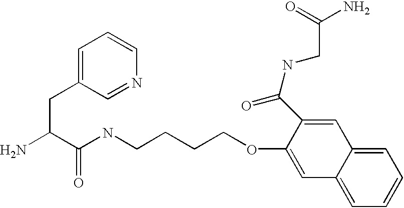 | 3-[[4-[[2(S)-AMINO-3-(PYRIDIN-3-YL)-1- OXO]PROPYL]AMINO]BUTOXY]- NAPHTHALENE-2-(N-ACETAMIDO)- CARBOXAMIDE | 9.6 |
| 49 | 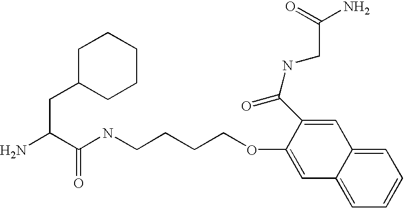 | 3-[[4-[[2(S)-AMINO-3(CYCLOHEXYL)-1- OXO]PROPYL]AMINO]BUTOXY]- NAPHTHALENE-2-(N-ACETAMIDO)- CARBOXAMIDE | 0.92 |
| 50 | 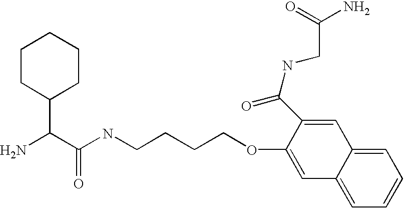 | 3-[[[4-(2(S)-AMINO-2-CYCLOHEXYL-1- OXO)ETHYL]AMINO]BUTOXY]- NAPHTHALENE-2-(N-ACETAMIDO)- CARBOXAMIDE | 2 |
| 51 | 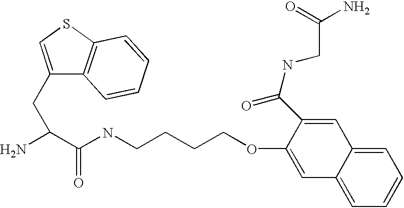 | 3-[[4-[[2(S)-AMINO-3-(BENZO- THIOPHEN-3-YL)-1-OXO]PROPYL]- AMINO]BUTOXY]-NAPHTHALENE-2- (N-ACETAMIDO)CARBOXAMIDE | 0.1 |
| 52 | 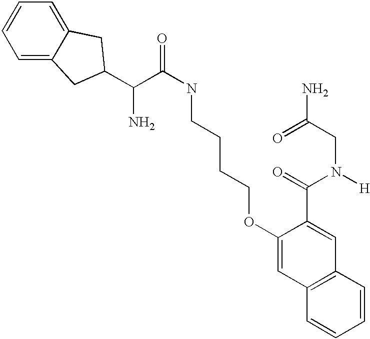 | 3-[4-[N-[1-OXO-2-AMINO-2-(INDAN-2- YL)]ETHYL]-AMINOBUTOXY]- NAPHTHALENE-2-(N-ACETAMIDO)- CARBOXAMIDE | 1.56 |
| 53 | 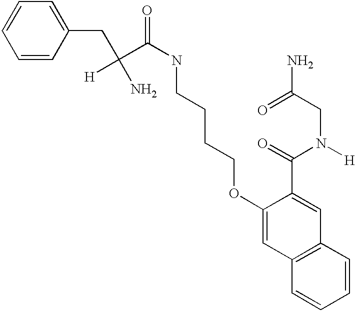 | 3-[4-[[[2(R)-AMINO-3-PHENYL-1- OXO]PROPYL]AMINO]BUTOXY]- NAPHTHALENE-2-(N-ACETAMIDO)- CARBOXAMIDE | 1.4 |
| 54 | 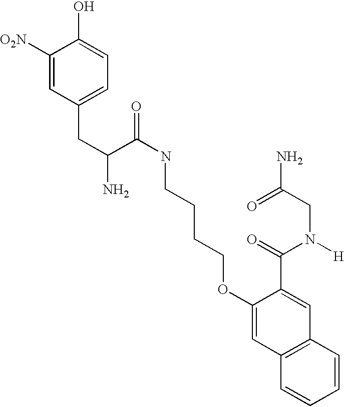 | 3-[4-[[[2(S)-AMINO-3-(3-NITRO-4- HYDROXY)PHENYL-1-OXO]PROPYL]- AMINO]BUTOXY]-NAPHTHALENE-2- (N-ACETAMIDO)CARBOXAMIDE | 0.66 |
| 55 | 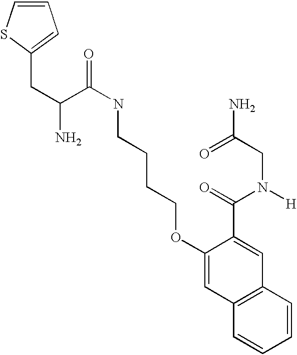 | 3-[4-[[[2(S)-AMINO-3-(THIOPHEN-2-YL)- 1-OXO]PROPYL]AMINO]BUTOXY]- NAPHTHALENE-2-(N-ACETAMIDO)- CARBOXAMIDE | 1.35 |
| 56 | 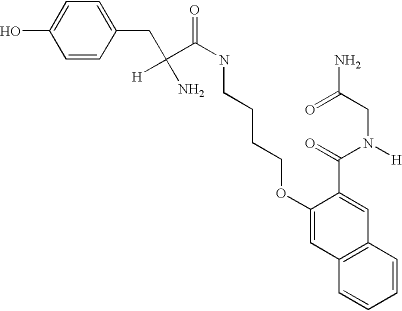 | 3-[4-[[[2(R)-AMINO-3-(4-HYDROXY- PHENYL-1-OXO]PROPYL]AMINO]- BUTOXY]-NAPHTHALENE-2-(N- ACETAMIDO)CARBOXAMIDE | 50 |
| 57 | 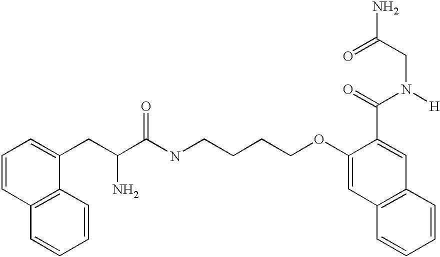 | 3-[4-[[[2(S)-AMINO-3-(NAPHTH-1-YL)-1- OXO]PROPYL]AMINO]BUTOXY]- NAPHTHALENE-2-(N-ACETAMIDO)- CARBOXAMIDE | 0.37 |
| 58 | 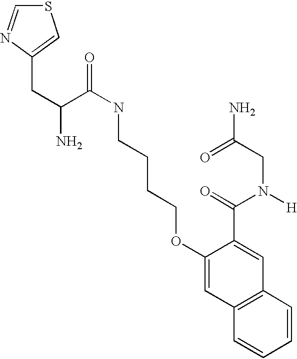 | 3-[4-[[[2(S)-AMINO-3-(THIAZOL-5-YL)-1- OXO]PROPYL]AMINO]BUTOXY]- NAPHTHALENE-2-(N-ACETAMIDO)- CARBOXAMIDE | 4.62 |
| 59 | 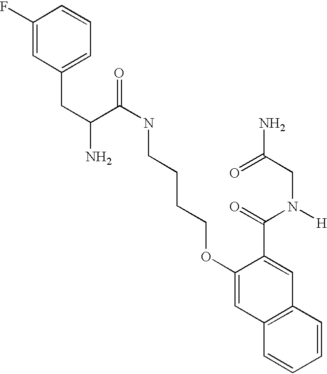 | 3-[4-[[[2(S)-AMINO-3-(3-FLUORO- PHENYL)-1-OXO]PROPYL]AMINO]- BUTOXY]-NAPHTHALENE-2-(N- ACETAMIDO)CARBOXAMIDE | 0.49 |
| 60 | 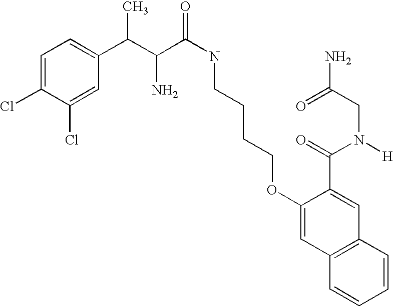 | 3-[4-[[[2(S)-AMINO-3(3,4-DICHLORO- PHENYL)-1-OXO]BUTYL]AMINO]- BUTOXY]-NAPHTHALENE-2-(N- ACETAMIDO)CARBOXAMIDE | 1.56 |
| 61 | 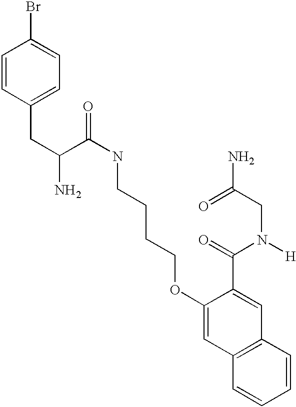 | 3-[4-[[[1-OXO-2(S)-AMINO-3(4- BROMOPHENYL)]PROPYL]AMINO]- BUTOXY]-NAPHTHALENE-2-(N- ACETAMIDO)CARBOXAMIDE | 0.5 |
| 62 | 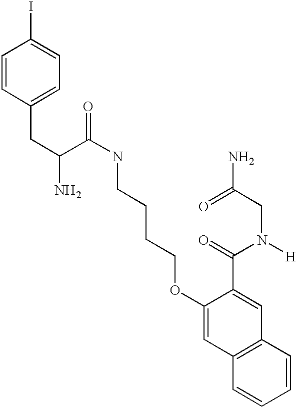 | 3-[4-[[[1-OXO-2(S)-AMINO-3(4- IODOPHENYL)]PROPYL]AMINO]- BUTOXY]-NAPHTHALENE-2-(N- ACETAMIDO)CARBOXAMIDE | 6.25 |
| 63 | 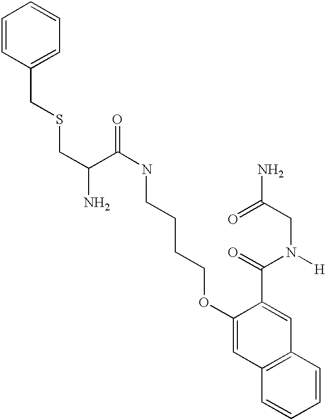 | 3-[4-[[[1-OXO-2(S)-AMINO- 3(BENZYLMERCAPTO)]PROPYL]- AMINO]BUTOXY]-NAPHTHALENE-2- (N-ACETAMIDO)CARBOXAMIDE | 2.23 |
| 64 | 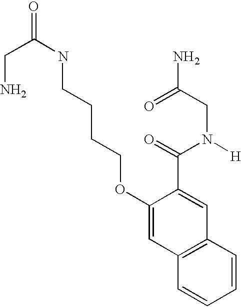 | 3-[4-[[(1-OXO-2-AMINO)ETHYL]- AMINO]BUTOXY]-NAPHTHALENE-2- (N-ACETAMIDO)CARBOXAMIDE | 68 |
| 65 | 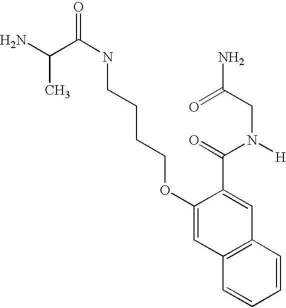 | 3-[4-[[(1-OXO-2(S)-AMINO)PROPYL]- AMINO]BUTOXY]-NAPHTHALENE-2- (N-ACETAMIDO)CARBOXAMIDE | 101 |
| 66 | 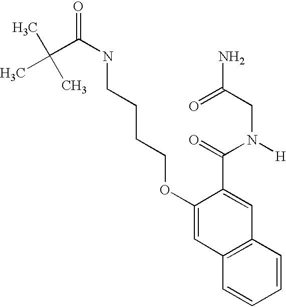 | 3-[4-[[(1-OXO-2,2-DIMETHYL)PROPYL]- AMINO]BUTOXY]-NAPHTHALENE-2- (N-ACETAMIDO)CARBOXAMIDE | 6 |
| 67 | 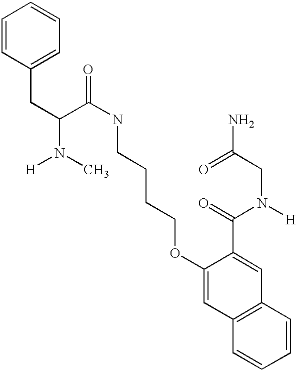 | 3-[4-[[(1-OXO-2(S)-METHYLAMINO-3- PHENYL)PROPYL]AMINO]BUTOXY]- NAPHTHALENE-2-(N-ACETAMIDO)- CARBOXAMIDE | 2.3 |
| 68 | 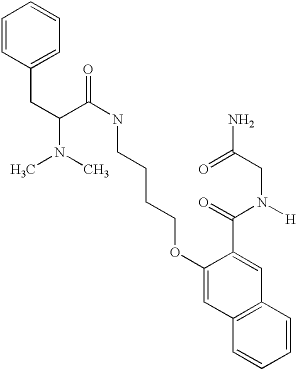 | 3-[4-[[(1-OXO-2(S)-N,N-DIMETHYL- AMINO)PROPYL]AMINO]BUTOXY]- NAPHTHALENE-2-(N-ACETAMIDO)- CARBOXAMIDE | 50 |
| 69 | 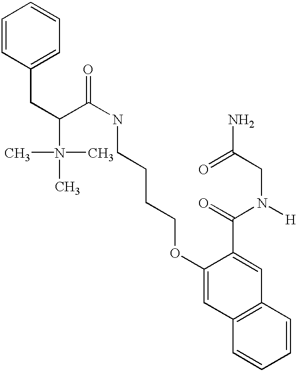 | 3-[4-[[(1-OXO-2(S)-TRIMETHYL- AMMONIUM)PROPYL]AMINO]- BUTOXY]-NAPHTHALENE-2-(N- ACETAMIDO)CARBOXAMIDE | 14 |
| 70 | 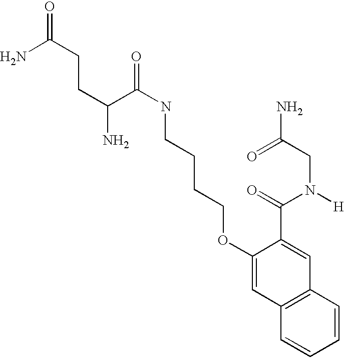 | 3-[4-[[(1-OXO-2(S)-AMINO-4-AMINO- CARBONYL)BUTYL]AMINO]BUTOXY]- NAPHTHALENE-2-(N-ACETAMIDO)- CARBOXAMIDE | 370 |
| 71 | 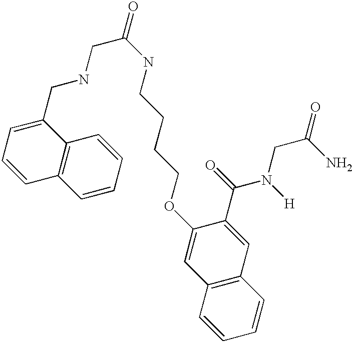 | 3-[4-[[1-OXO-3-[[[NAPHTH-1-YL]- METHYL](S)AMINO]PROPYL]AMINO]- BUTOXY]-NAPHTHALENE-2-(N- ACETAMIDO)CARBOXAMIDE | 6 |
| 72 | 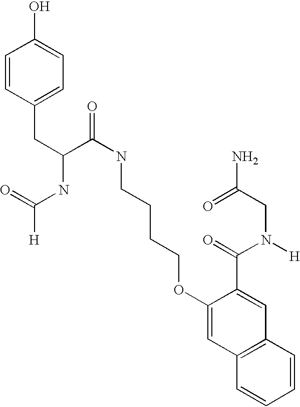 | 3-[4-[[[1-OXO-2-(N-FORMYLAMINO)-3- (4-HYDROXYPHENYL)]PROPYL]- (S)AMINO]BUTOXY]-NAPHTHALENE- 2-(N-ACETAMIDO)CARBOXAMIDE | 6 |
| 73 | 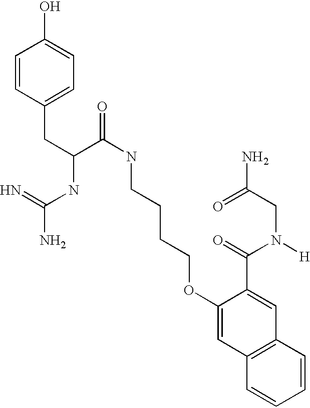 | 3-[4-[[[(1-OXO-2-[(N-AMINOIMINO- METHYL)(S)AMINO]-3-(4-HYDROXY- PHENYL)]PROPYL]AMINO]BUTOXY]- NAPHTHALENE-2-(N-ACETAMIDO)- CARBOXAMIDE | 6 |
| 74 | 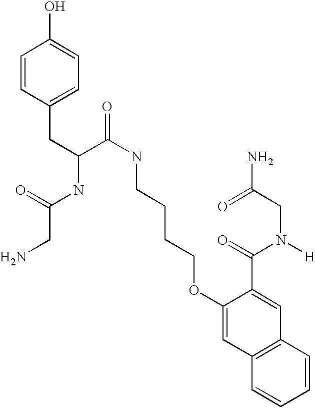 | 3-[4-[[[(1-OXO-2-[(N-AMINOMETHYL- CARBONYL)(S)AMINO]3-(4-HYDROXY- PHENYL)]PROPYL]AMINO]BUTOXY]- NAPHTHALENE-2-(N-ACETAMIDO)- CARBOXAMIDE | 25 |
| 75 | 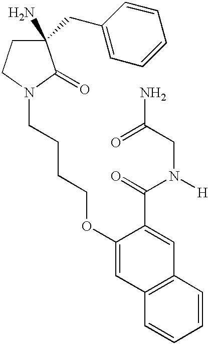 | 3-[4-[(2-OXO-3(R)-AMINO-3-BENZYL)- PYRROLIDIN-1-YL]BUTOXY]- NAPHTHALENE-2-(N-ACETAMIDO)- CARBOXAMIDE | 6 |
| 76 | 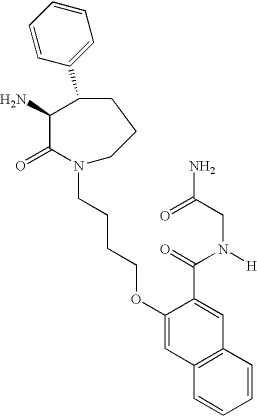 | 3-[4-[(2-OXO-3(R)-AMINO-4(S)- PHENYL)HEXAMETHYLENEIMIN-1- YL]BUTOXY]-NAPHTHALENE-2-(N- ACETAMIDO)CARBOXAMIDE | 6 |
| 77 | 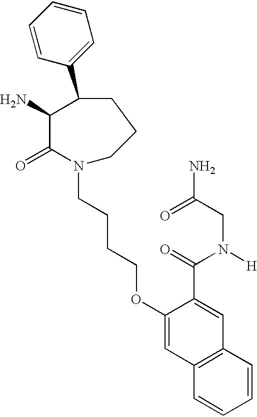 | 3-[4-[(2-OXO-3(R)-AMINO-4(R)- PHENYL)HEXAMETHYLENEIMIN-1- YL]BUTOXY]-NAPHTHALENE-2-(N- ACETAMIDO)CARBOXAMIDE | 6 |
| 78 | 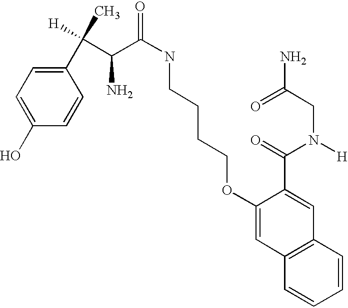 | 3-[4-[[[(1-OXO-2-AMINO-3(R)-METHYL- 3-(4-HYDROXY)PHENYL)]PROPYL]- AMINO]BUTOXY]-NAPHTHALENE-2- (N-ACETAMIDO)CARBOXAMIDE | 6 |
| 79 | 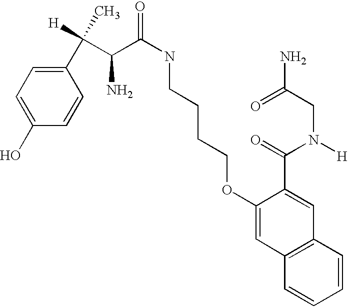 | 3-[4-[[[(1-OXO-2(S)-AMINO-3(R)- METHYL-3-(4-HYDROXYPHENYL)]- PROPYL]AMINO]BUTOXY]- NAPHTHALENE-2-(N-ACETAMIDO)- CARBOXAMIDE | 6 |
| 80 | 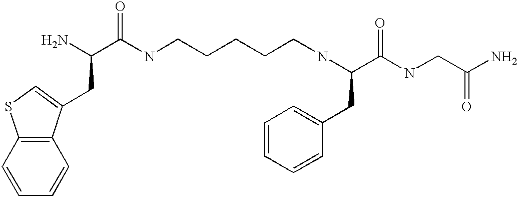 | N-[((R)BENZYL)[(AMINO)(CARBONYL) (METHYL)(AMINO)(CARBONYL)]- METHYL]-5-[[N-[1-OXO-2- AMINO-3-(R)(BENZOTHIOPHEN-3-YL)]PROPYL]AMINO]PENTYLAMINE | 6 |
| 81 | 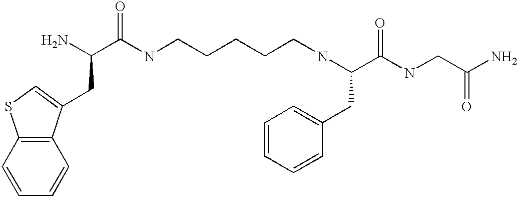 | N-[((S)BENZYL)[(AMINO)(CARBONYL)- (METHYL)(AMINO)(CARBONYL)]- METHYL]-5-[[N-[1-OXO-2-AMINO-3- (R)(BENZOTHIOPHEN-3- YL)]PROPYL]AMINO]PENTYLAMINE | 6 |
| 82 | 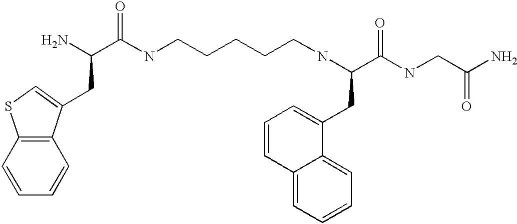 | N-[((R)NAPHTH-1-YL)[(AMINO)- (CARBONYL)(METHYL)(AMINO)- (CARBONYL)]METHYL]-5-[N-[[N-[1- OXO-2-AMINO-3-(R)(BENZOTHIOPHEN- 3-YL)]PROPYL]AMINO]PENTYLAMINE | 6 |
| 83 | 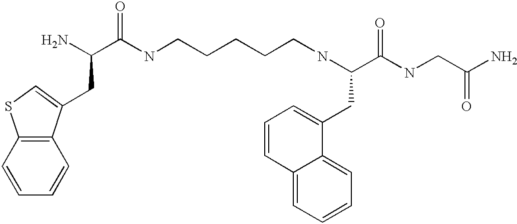 | N-[((S)NAPHTH-1-YL)METHYL]- [(AMINO)(CARBONYL)(METHYL)- (AMINO)(CARBONYL)]METHYL]-5-[N- [[(1-OXO-2-AMINO-3-(R)(BENZO- THIOPHEN-3-YL)]PROPYL]AMINO]- PENTYLAMINE | 100 |
| 84 | 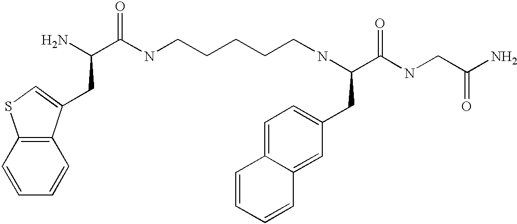 | N-[[(R)(NAPHTH-2-YL)METHYL]- [(AMINO)(CARBONYL)(METHYL)- (AMINO)(CARBONYL)]METHYL]-5-[N- [[[(1-OXO-2-AMINO-3-(R)(BENZO- THIOPHEN-3-YL)]PROPYL]AMINO]- PENTYLAMINE | 100 |
| 85 | 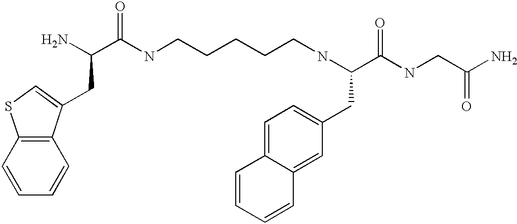 | N-[[(S)(NAPHTH-2-YL)METHYL]- [(AMINO)(CARBONYL)(METHYL)- (AMINO)(CARBONYL)]METHYL]-5-[N- [[(1-OXO-2-AMINO-3-(R)(BENZO- THIOPHEN-3-YL)PROPYL]AMINO]]- PENTYLAMINE | 100 |
| 86 | 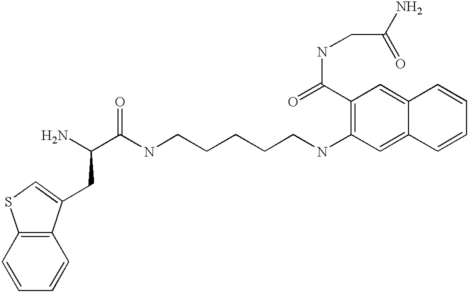 | 3-[N-[5-[[(1-OXO-2-AMINO-3-(BENZO- THIOPHEN-3-YL)PROPYL]AMINO]- PENTYL]AMINO]-NAPHTHALENE-2-(N- ACETAMIDO)CARBOXAMIDE | 100 |
| 87 | 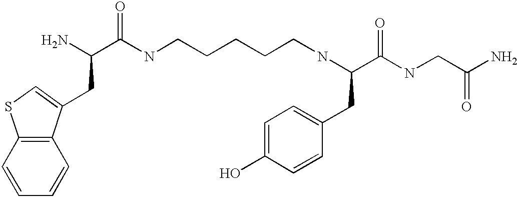 | N-[[(R)4-(METHYL)(S)(AMINO)- (CARBONYL)]METHYL]-5-[N-[(1-2- AMINO-3-(R)-(BENZOTHIOPHEN-3- PENTYLAMINE | 100 |
| 88 | 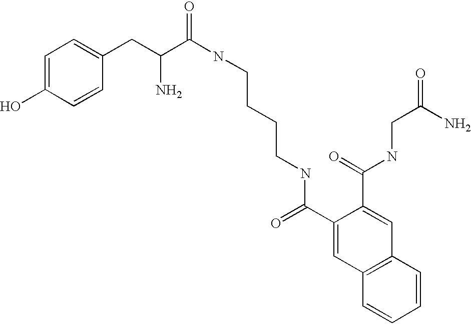 | 3-[[[4-[[[1-OXO-2(S)-AMINO-3- HYDROXYPHENYL)]PROPYL]AMINO]- BUTYL]AMINO]CARBONYL]- NAPHTHALENE-2-ACETAMIDO)- CARBOXAMID | 400 |
| 89 | 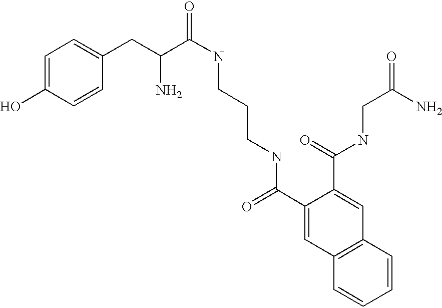 | 3-[[[3-[[[1-OXO-2(S)-AMINO-3- HYDROXYPHENYL)]PROPYL]AMINO]- PROPYL]AMINO]CARBONYL]- NAPHTHALENE-2-ACETAMIDO)- CARBOXAMID | 252 |
| 90 | 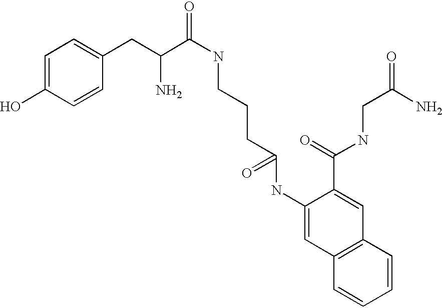 | 3-[[[3-[[[1-OXO-2(S)-AMINO-3- HYDROXYPHENYL)]PROPYL]AMINO]- PROPYL]CARBONYL]AMINO]- NAPHTHALENE-2-ACETAMIDO)- CARBOXAMID | 209 |
| 91 | 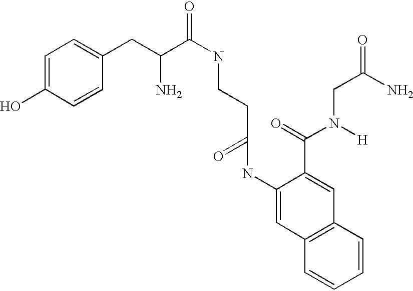 | 3-[[[4-[[[1-OXO-2(S)-AMINO-3- HYDROXYPHENYL)]PROPYL]AMINO]- BUTYL]CARBONYL]AMINO]- NAPHTHALENE-2-ACETAMIDO)- CARBOXAMIDE | 209 |
| 92 | 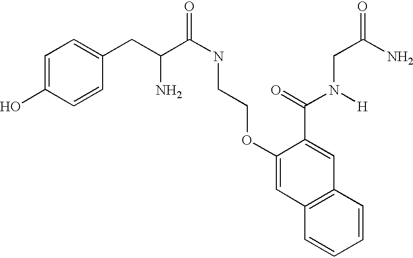 | 3-[2-[[[1-OXO-2(S)-AMINO-3-HYDROXY- PHENYL)]PROPYL]AMINO]ETHOXY]NAPHTHALENE-2-(N- | 3.4 |
| 93 | 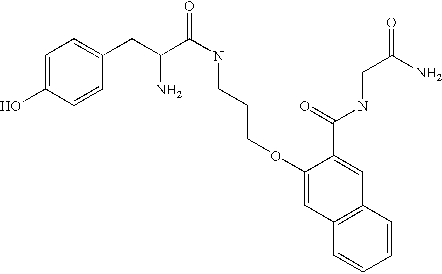 | 3-[5-[[[1-OXO-2(S)-AMINO-3-(4- HYDROXYPHENYL)]PROPYL]AMINO]- PROPYLOXY]NAPHTHALENE-2-(N- ACETAMIDO)CARBOXAMIDE | 45 |
| 94 | 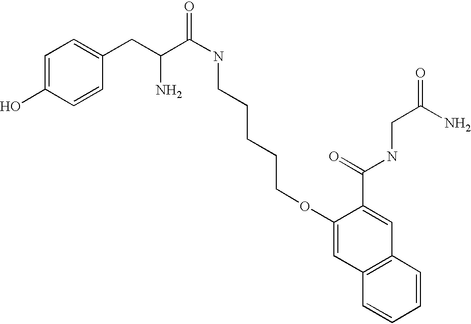 | 3-[5-[[[1-OXO-2(S)-AMINO-3-(4- HYDROXYPHENYL)]PROPYL]AMINO]- PENTOXY]NAPHTHALENE-2-(N- ACETAMIDO)CARBOXAMIDE | 6.6 |
| 95 | 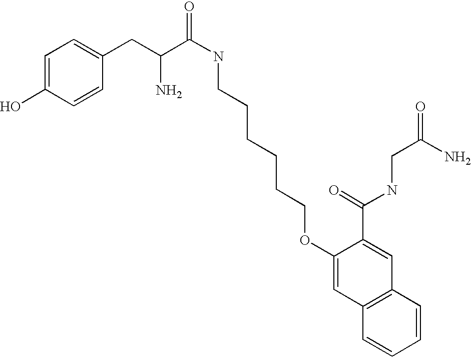 | 3-[6-[[[1-OXO-2(S)-AMINO-3-(4- HYDROXYPHENYL)]PROPYL]AMINO]- HEXOXY]NAPHTHALENE-2-(N- ACETAMIDO)CARBOXAMIDE | 7.1 |
| 96 | 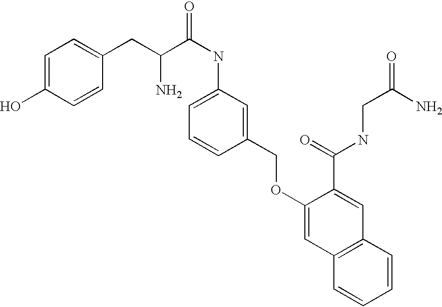 | 3-[3-[[[1-OXO-2(S)-AMINO-3-(4- HYDROXYPHENYL)]PROPYL]AMINO]- BENZYLOXY]NAPHTHALENE-2-(N- ACETAMIDO)CARBOXAMIDE | 7.1 |
| 97 | 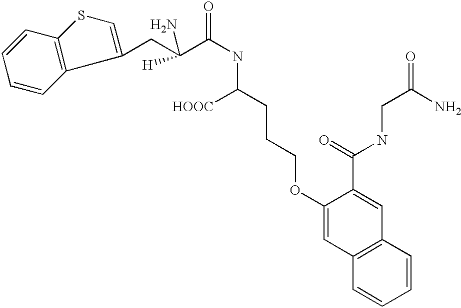 | 3-[[4-[[1-OXO-2(S)-AMINO-3-(BENZO- THIOPHEN-3-YL)]PROPYL]AMINO][4- CARBOXY]BUTOXY]NAPHTHALENE-2- (N-ACETAMIDO)CARBOXAMIDE | 26 |
| 98 | 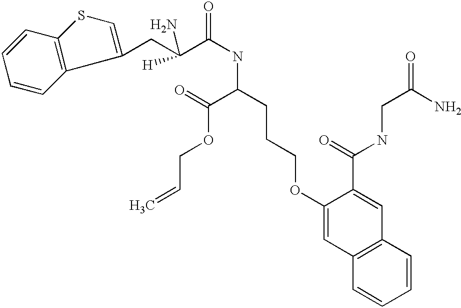 | 3-[[4-[[1-OXO-2(S)-AMINO-3-(BENZO- THIOPHEN-3-YL)]PROPYL]AMINO][4- ACRYLOXYCARBOXY]BUTOXY]NAPHTHALENE-2-(N-ACETAMIDO)- CARBOXAMIDE | 2.5 |
| 99 | 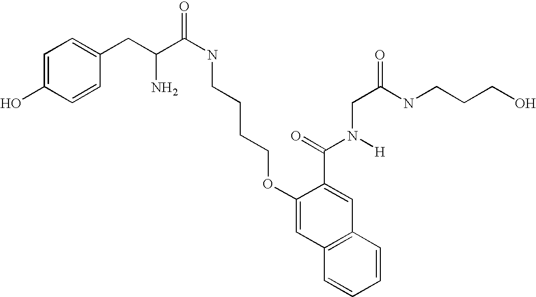 | 3-[[4-[[1-OXO-2(S)-AMINO-3-(4- | 0.49 HYDROXYPHENYL)]PROPYL]AMINO]- BUTOXY]NAPHTHALENE-2-[N-[[[[3- HYDROXYPROPYL]AMINO]- CARBONYL]METHYL]]CARBOXAMIDE |
| 100 | 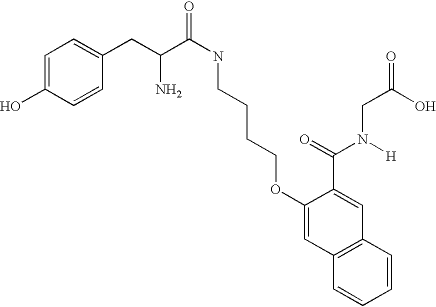 | 3-[[4-[[1-OXO-2(S)-AMINO-3-(4- HYDROXYPHENYL)]PROPYL]AMINO]- BUTOXY]NAPHTHALENE-2-[N- CARBOXYMETHYL]CARBOXAMIDE | 100 |
| 101 | 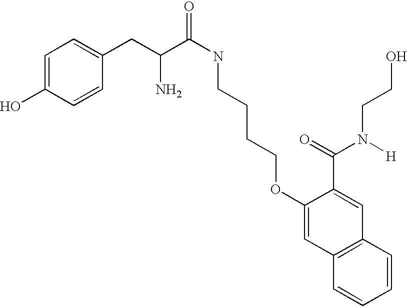 | 3-[[4-[[1-OXO-2(S)-AMINO-3-(4- HYDROXYPHENYL)]PROPYL]AMINO]- BUTOXY]NAPHTHALENE-2-[N- (2-HYDROXYETHYL)]CARBOXAMIDE | 15 |
| 102 | 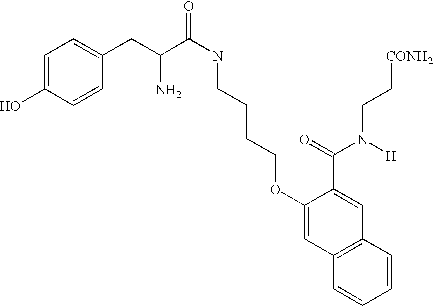 | 3-[[4-[[1-OXO-2(S)-AMINO-3-(3,4- DIHYDROXYPHENYL)]PROPYL]- AMINO]BUTOXY]NAPHTHALENE-2-(N- ACETAMIDOMETHYL)CARBOXAMIDE | 52 |
| 103 | 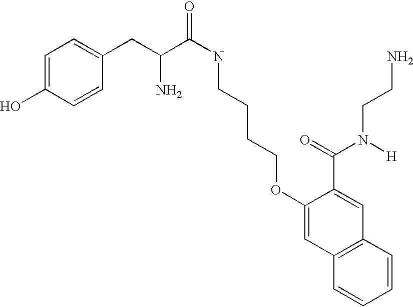 | 3-[[4-[[1-OXO-2(S)-AMINO-3-(4- HYDROXYPHENYL)]PROPYL]AMINO]- BUTOXY]NAPHTHALENE-2-[N-(2- AMINOETHYL)]CARBOXAMIDE | 400 |
| 104 | 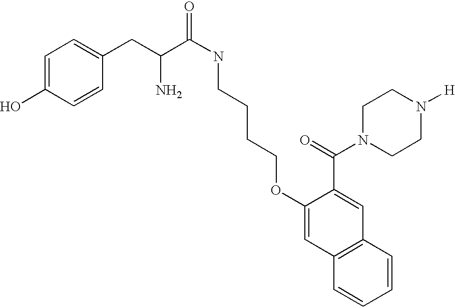 | 3-[[4-[[2(S)-AMINO-3-(4-HYDROXY- PHENYL)-1-OXOPROPYL]AMINO]- BUTOXY]-NAPHTHALENE-2-[N,N- PIPERAZIN-1-YL]CARBOXAMIDE | 400 |
| 105 | 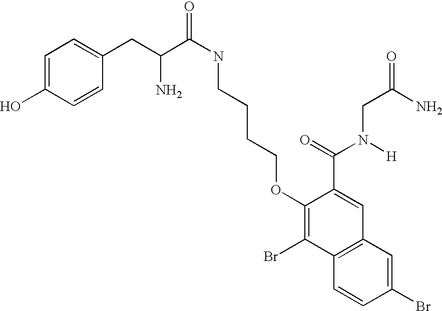 | 1,6-DIBROMO-2-[[4-[[1-OXO-2(S)- AMINO-3-(4-HYDROXYPHENYL)]- PROPYL]AMINO]BUTOXY]- NAPHTHALENE-3-(N-ACETAMIDO)- CARBOXAMIDE | 20 |
| 106 | 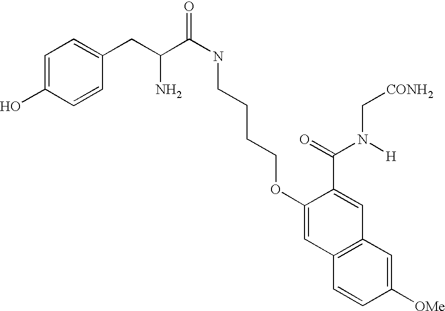 | 3-[[4-[[2(S)-AMINO-3-(4-HYDROXY- PHENYL)-1-OXOPROPYL]AMINO]- BUTOXY]-7-METHOXY- NAPHTHALENE-2-[N-ACETAMIDO]- CARBOXAMIDE | 2.5 |
| 107 | 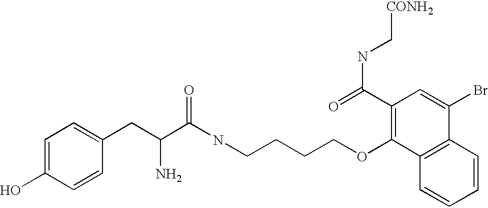 | 1-BROMO-4-[[4-[[1-OXO-2-AMINO-3-(4- HYDROXYPHENYL)]PROPYL]- (S)AMINO]BUTOXY]NAPHTHALENE-3- (N-ACETAMIDO)CARBOXAMIDE | 100 |
| 108 | 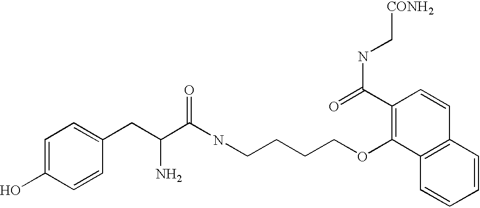 | 1-[[4-[[1-OXO-2(S)-AMINO-3-(4- HYDROXYPHENYL)]PROPYL]AMINO]BUTOXY]NAPHTHALENE-3-(N- ACETAMIDO)CARBOXAMIDE | 100 |
| 109 | 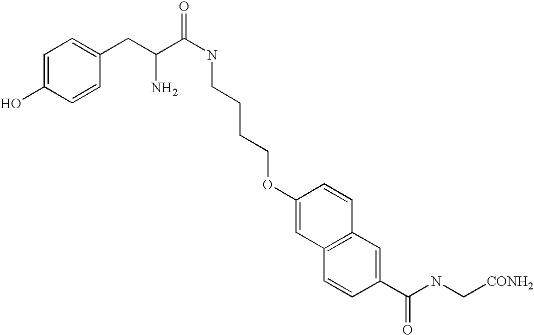 | 2-[[4-[[1-OXO-2(S)-AMINO-3-(4- HYDROXYPHENYL)]PROPYL]AMINO]BUTOXY]NAPHTHALENE-6-(N- ACETAMIDO)CARBOXAMIDE | 100 |
| 110 | 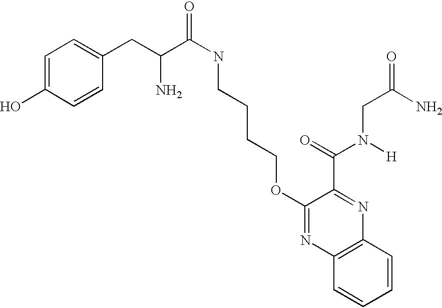 | 3-[4-[N-[1-OXO-2(S)-AMINO-3-(4- HYDROXYPHENYL)]PROPYL]-AMINO- BUTOXY]-QUINOXALINE-2-(N- ACETAMIDO)CARBOXAMIDE | 400 |
| 111 | 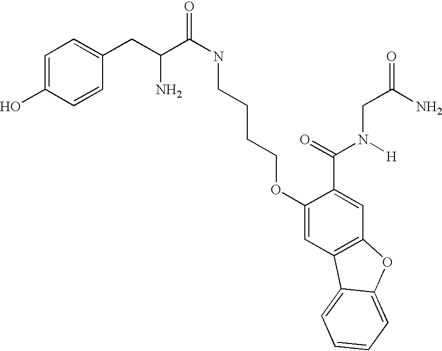 | 4-[[4-[[1-OXO-2(S)-AMINO-3-(4- HYDROXYPHENYL)]PROPYL]AMINO]BUTOXY]DIBENZOFURAN-3-(N- ACETAMIDO)CARBOXAMIDE | 100 |
| 112 | 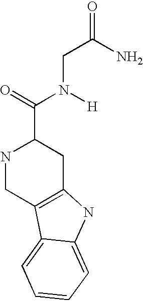 | 1,2,3,4-TETRAHYDROCARBOLINE-3-(N- ACETAMIDO)CARBOXAMIDE | 400 |
| 113 | 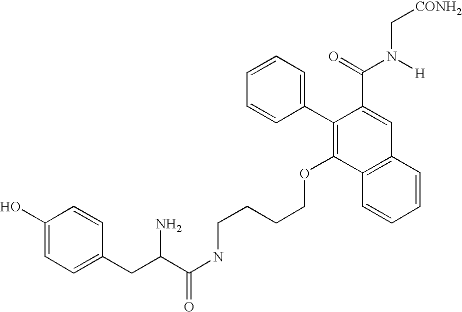 | 1-[[4-[[1-OXO-2(S)-AMINO-3-(4- HYDROXYPHENYL)]PROPYL]AMINO]BUTOXY]-2-PHENYL-NAPHTHALENE- 3-(N-ACETAMIDO)CARBOXAMIDE | 400 |
| 114 | 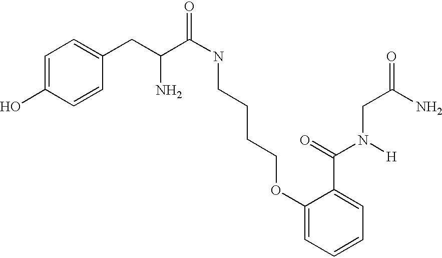 | 3-[[4-[[1-OXO-2(S)-AMINO-3-(4- HYDROXYPHENYL)]PROPYL]AMINO]BUTOXY]PYRIDINE-2-(N- ACETAMIDO)CARBOXAMIDE | 400 |
| 115 | 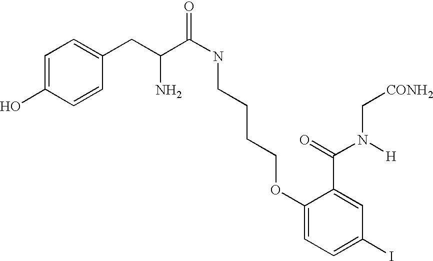 | 1-IODO-4-[[4-[[1-OXO-2(S)-AMINO-3-(4- HYDROXYPHENYL)]PROPYL]AMINO]BUTOXY](N-ACETAMIDO)- CARBOXAMIDE | 100 |
| 116 | 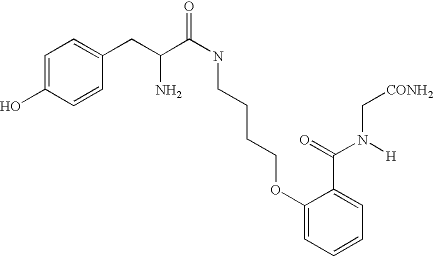 | 2-[[4-[[1-OXO-2(S)-AMINO-3-(4- HYDROXYPHENYL)]PROPYL]AMINO]BUTOXY]BENZENE-(N-ACETAMIDO)- CARBOXAMIDE | 400 |
| 117 | 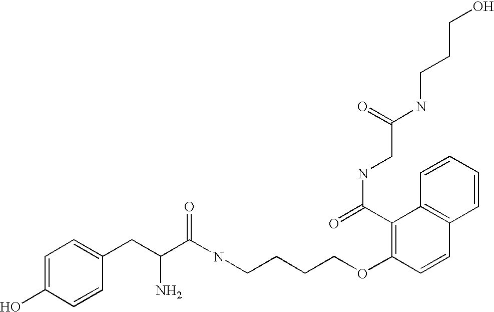 | 2-[[4-[[1-OXO-2(S)-AMINO-3-(4- HYDROXYPHENYL)]PROPYL]AMINO]BUTOXY]NAPHTHALENE-1-[N-[[(3- HYDROXYPROPYLAMINO)- CARBONYL]METHYL]]CARBOXAMIDE | 47 |
| 118 | 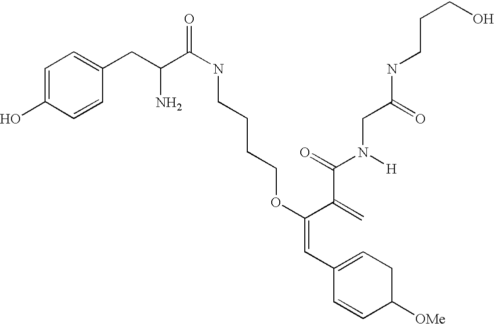 | 3-[[4-[[1-OXO-2(S)-AMINO-3-(4- HYDROXYPHENYL)]PROPYL]AMINO]BUTOXY]-7-METHOXY- NAPHTHALENE-2-[N-[[(3-HYDROXY- PROPYLAMINO)-CARBONYL]- METHYL]]CARBOXAMIDE | 47 |
| 119 | 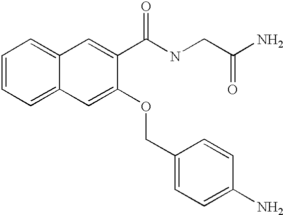 | 2-[4-AMINOBENZYLOXY]- NAPHTHALENE-3-[N-ACETAMIDO]- CARBOXAMIDE | 25 |
| 120 | 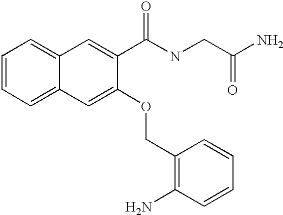 | 2-[2-AMINOBENZYLOXY]- NAPHTHALENE-3-[N-ACETAMIDO]- CARBOXAMIDE | 100 |
| 121 | 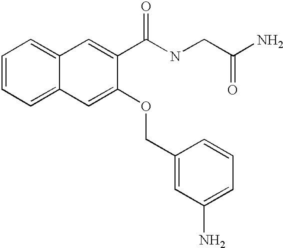 | 2-[3-AMINOBENZYLOXY]- NAPHTHALENE-3-[N-ACETAMIDO]- CARBOXAMIDE | 100 |
| 122 | 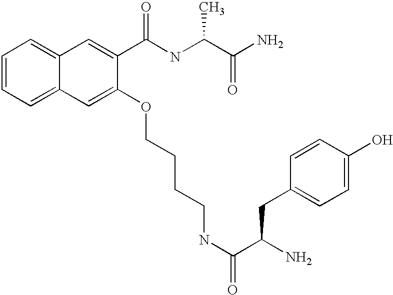 | 2-[[4-[[1-OXO-2-AMINO-3-(4-HYDROXY- PHENYL)]PROPYL]AMINO] BUTOXY]- NAPHTHALENE-3-[N-[1(S)-METHYL-2- OXO-2-AMINO)ETHYL]ACETAMIDO]- CARBOXAMIDE | 12 |
| 123 | 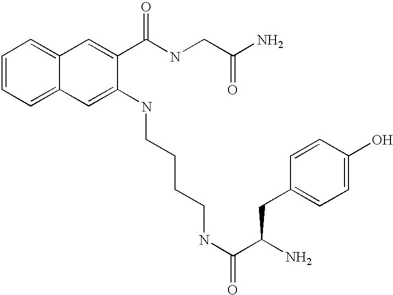 | 2-[[4-[[1-OXO-2(S)-AMINO-2(R)-(4- HYDROXYBENZYL)]ETHYL]AMINO]BUTOXY]-NAPHTHALENE-3-[N- ACETAMIDO]CARBOXAMIDE | 25 |
| 124 | 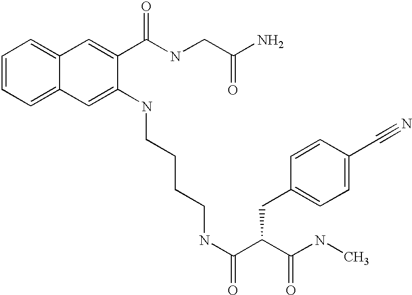 | 2-[[4-[[1-OXO-2-(N-METHYLAMINO- CARBONYL)-2-(S)-(4-CYANOBENZYL)]- ETHYL]AMINO]BUTOXY]- NAPHTHALENE-3-[N-ACETAMIDO]- CARBOXAMIDE | 6 |
| 125 | 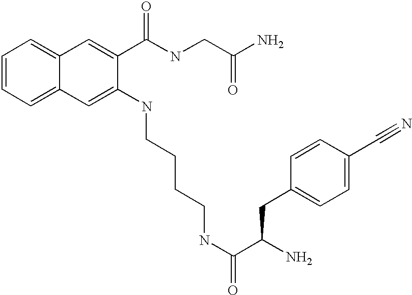 | 3-[[4-[[1-OXO-2-AMINO-2(S-(4- CYANOBENZYL)]ETHYL]AMINO]BUTOXY]-NAPHTHALENE-2-[N- ACETAMIDO]CARBOXAMIDE | 3 |
| 126 | 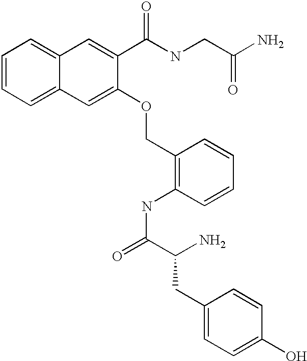 | 3-[[2-[1-OXO-2-AMINO-2(S)-(4- HYDROXYBENZYL)ETHYL]AMINO]- BENZYLOXY]-NAPHTHALENE-2-[N- ACETAMIDO]CARBOXAMIDE | 100 |
| 127 | 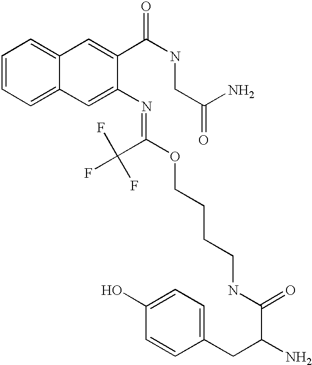 | 3-[[1-TRIFLUOROMETHYL][1-[4-[[[1- OXO-2-AMINO-3-HYDROXYPHENYL)]- PROPYL]AMINO]BUTOXY]]- METHYLENIMINO]-NAPHTHALENE-2- [N-ACETAMIDO]CARBOXAMIDE | 19 |
| 128 | 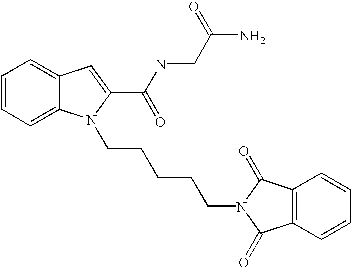 | 1-[5-(PHTHALIMID-1-YL)PENTYL]- INDOLE-2-ACETAMIDO)- CARBOXAMIDE | 400 |
| 129 | 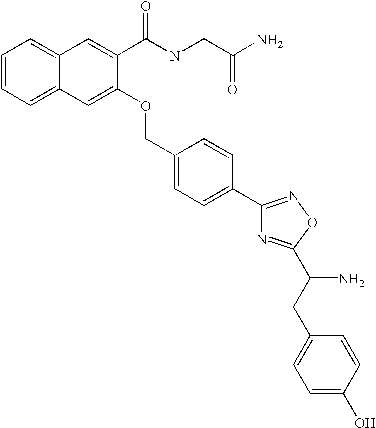 | 3-[4-[5-[1-AMINO-2-(4-OXADIAZOL-3- YL]BENZYLOXY]-NAPHTHALENE-2- ACETAMIDO)CARBOXAMIDE | 100 |
| 130 | 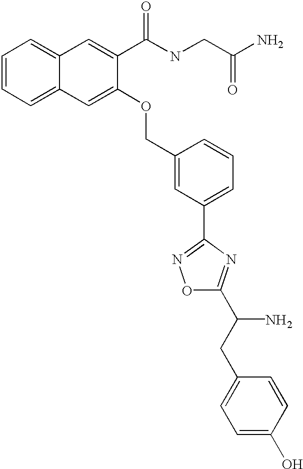 | 3-[3-[5-[1-AMINO-2-(4-HYDROXY- PHENYL)ETHYL]1,2,4-OXADIAZOL-3- YL]BENZYLOXY]-NAPHTHALENE-2-(N- ACETAMIDO)CARBOXAMIDE | 100 |
| 131 | 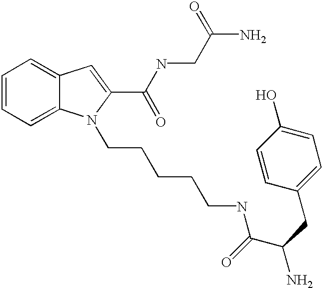 | 1-[5-[N-[1-OXO-2-AMINO-3(S)-(4- HYDROXYPHENYL)]PROPYL]-AMINO- PENTYL]-INDOLE-2-(N-ACETAMIDO)- CARBOXAMIDE | 100 |
| 132 | 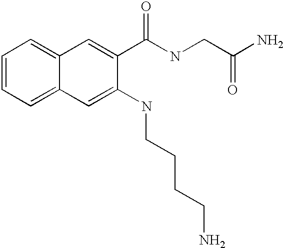 | 2-[N-(4-AMINOBUTYL)AMINO]- NAPHTHALENE-3-[N-ACETAMIDO]- CARBOXAMIDE | 100 |
| 133 | 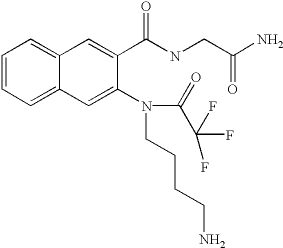 | 2-[[N-(4-AMINOBUTYL)-N-(TRI- FLUOROACETYL)]AMINO]- NAPHTHALENE-3-[N-ACETAMIDO]- CARBOXAMIDE | 100 |
| 134 | 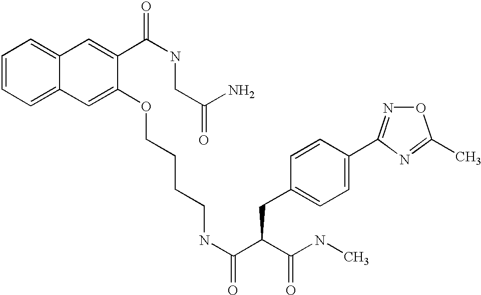 | 3-[[4-[N-[1-OXO-2-(N-METHYLCARBOX- AMIDO)-3-[4-(5-METHYL-1,2,4- OXADIAZOL-3-YL)PHENYL]]PROPYL]- AMINO] BUTOXY]-NAPHTHALENE-2- [N-ACETAMIDO]CARBOXAMIDE | 76 |
| 135 | 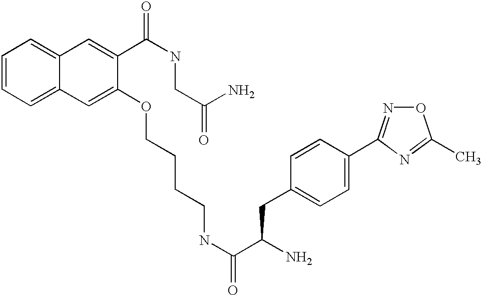 | 3-[[4-[N-[1-OXO-2-AMINO-3-[4-(5- METHYL-1,2,4-OXADIAZOL-3-YL)- PHENYL]]PROPYL]AMINO] BUTOXY]- NAPHTHALENE-2-[N-ACETAMIDO]- CARBOXAMIDE | 29 |
| 136 | 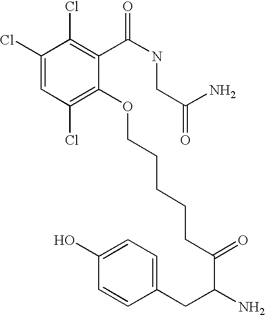 | 2-[4[N-[1-OXO-2-AMINO-3-(4- HYDROXYPHENYL)PROPYL]AMINO]- BUTOXY]-3,5,6-TRICHLORO-BENZENE- 1-[N-ACETAMIDO]CARBOXAMIDE | 200 |
| 137 | 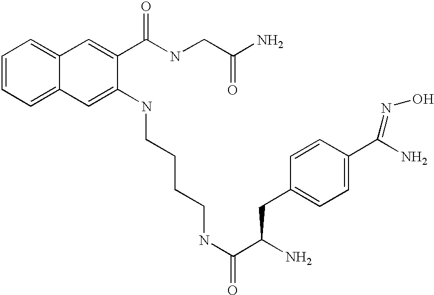 | 3-[4[N-[1-OXO-2-AMINO-3(S)-[4- [(HYDROXYIMINO)(AMINO)METHYL]- PHENYL]PROPYL]AMINO]BUTOXY]- NAPHTHALENE-2-[N- ACETAMIDO]CARBOXAMIDE | 13 |
| 138 | 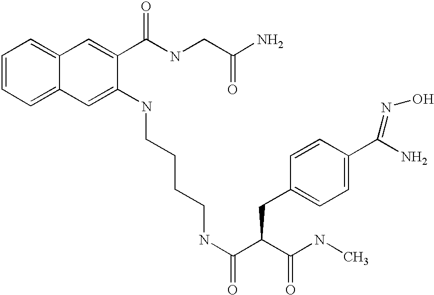 | 3-[4[N-[1-OXO-2-(METHYLAMINO- CARBONYL)-3-[4-[(HYDROXYIMINO)- (AMINO)METHYL]PHENYL]- PROPYL]AMINO]BUTOXY]- NAPHTHALENE-2-[N-ACETAMIDO]- CARBOXAMIDE | 38 |
| 139 | 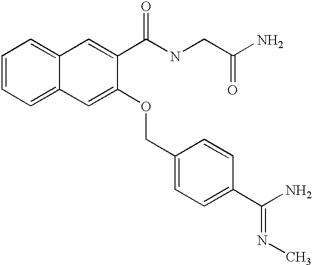 | 3-[[4-NAPHTHALENE-2-[N- | 100 |
| 140 | 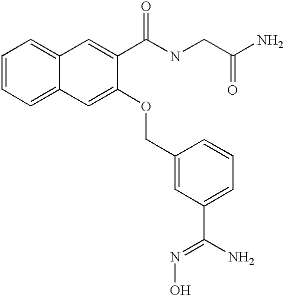 | 3-[[3-NAPHTHALENE-2-[N- | 100 |
| 141 | 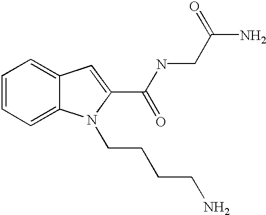 | 1-(4-AMINOBUTYL)INDOLE-2- ACETAMIDO]CARBOXAMID | 100 |
| 142 | 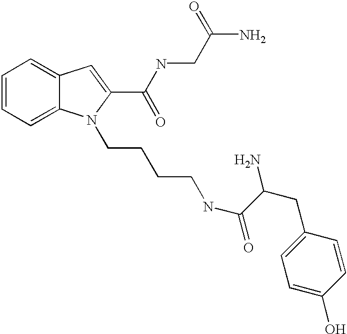 | 1-[[4-[N-[1-OXO-2-AMINO-3- HYDROXYPHENYL)]PROPYL]AMINO]- BUTYL]-INDOLE-2-ACETAMIDO)- CARBOXAMID | 100 |
| 143 | 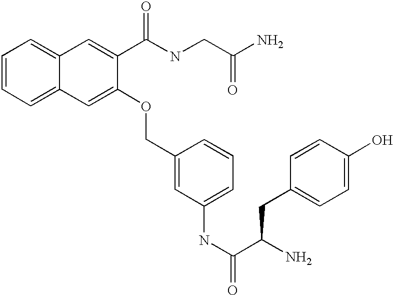 | 2-[[3-[N-[1-OXO-2-AMINO-3-(4- HYDROXYPHENYL)]PROPYL]AMINO]BENZYLOXY]-NAPHTHALENE-3-[N- ACETAMIDO]CARBOXAMIDE | 100 |
| 144 | 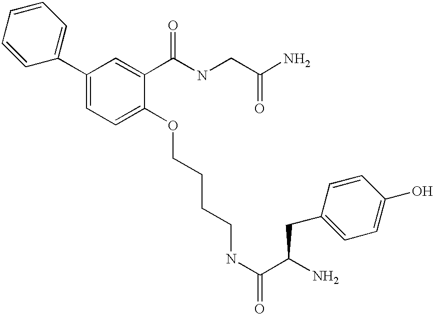 | 2-[[4-[N-[1-OXO-2-AMINO-3-(4- HYDROXYPHENYL)]PROPYL]AMINO]- BUTOXY]-5-PHENYL-BENZENE- (N-ACETAMIDO)CARBOXAMIDE | 30 |
| 145 | 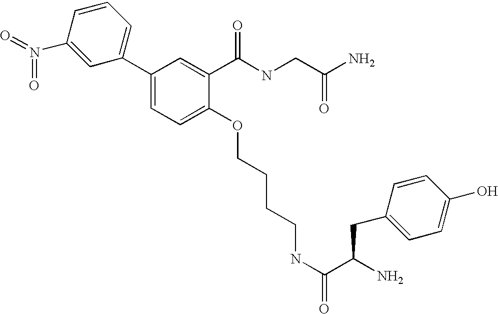 | 2-[[4-[N-[1-OXO-2-AMINO-3-(4- HYDROXYPHENYL)]PROPYL]AMINO]- BUTOXY]-5-(3-NITROPHENYL)- BENZENE- (N-ACETAMIDO)- CARBOXAMIDE | 100 |
| 146 | 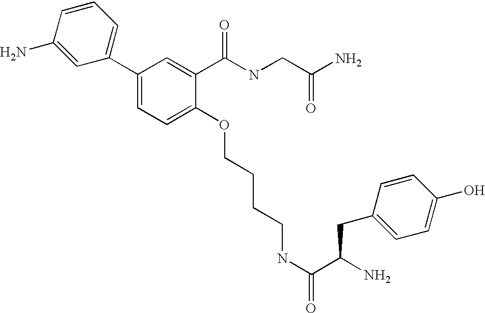 | 2-[[4-[N-[1-OXO-2-AMINO-3-(4- HYDROXYPHENYL)]PROPYL]AMINO]- BUTOXY]-5-(3-AMINOPHENYL)- BENZENE- (N-ACETAMIDO)- CARBOXAMIDE | 100 |
| 147 | 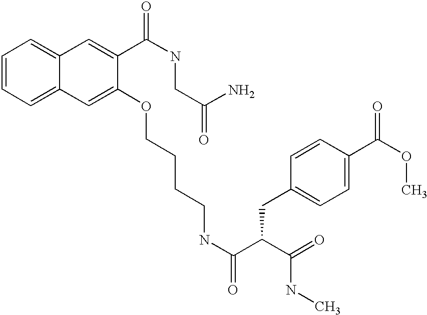 | 3-[[4-[N-[1-OXO-2-(N-METHYLAMINO- CARBONYL)-3-[(4-(METHOXY- CARBONYL)PHENYL]]PROPYL]- AMINO]BUTOXY]-NAPHTHALENE-2- (N-ACETAMIDO)CARBOXAMIDE | 18 |
| 148 | 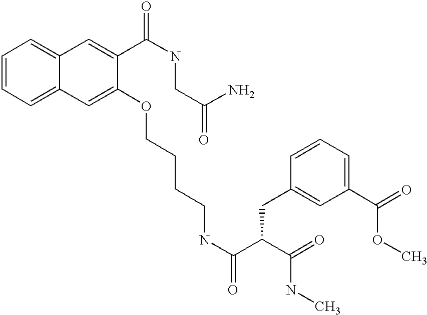 | 3-[[4-[N-[1-OXO-2-(N-METHYLAMINO- CARBONYL)-3-[(3-(METHOXY- CARBONYL)PHENYL]]PROPYL]- AMINO]BUTOXY]-NAPHTHALENE-2- (N-ACETAMIDO)CARBOXAMIDE | 203 |
| 149 | 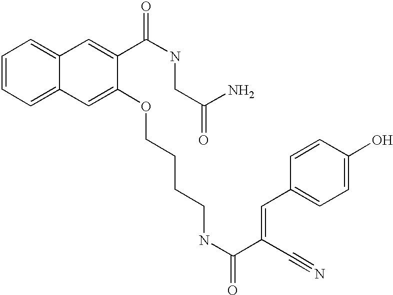 | 3-[[4-[N-[1-OXO-2-(CYANO)-3-(4- HYDROXYPHENYL)]PROPEN-2-YL]- AMINO] BUTOXY]-NAPHTHALENE-2- [N-ACETAMIDO]CARBOXAMIDE | 27 |
| 150 | 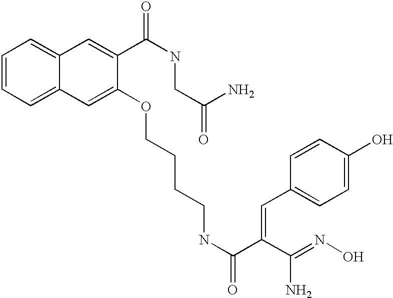 | 3-[[4-[N-[1-OXO-2-[(HYDROXYIMINO)- (AMINO)METHYL]-3-(4-HYDROXY- PHENYL)]]PROP-2-ENYL]AMINO]- BUTOXY]-NAPHTHALENE-2-(N- ACETAMIDO)CARBOXAMIDE | 16 |
| 151 | 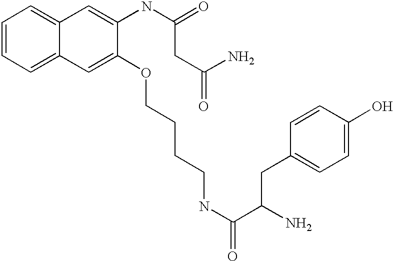 | 2-[(1,3-DIOXO-3-AMINO)PROPYL]- AMINO-3-[[4-[N-[1-OXO-2(S)-(AMINO)- 3-(4-HYDROXYPHENYL)]PROPYL]- AMINO]BUTOXY]-NAPHTHALENE | 5 |
| 152 | 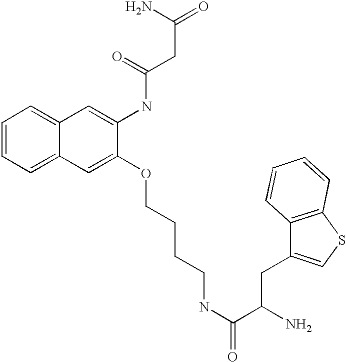 | 2-[(1,3-DIOXO-3-AMINO)PROPYL]- AMINO-3-[[4-[N-[1-OXO-2(S)-AMINO-3- (BENZOTHIOPHEN-3-YL)]PROPYL]- AMINO]BUTOXY]-NAPHTHALENE | 1 |
| 153 | 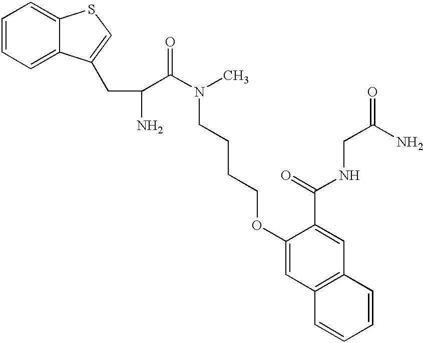 | 3-[[[4-[3-(BENZOTHIOPHEN-3-YL)-2- AMINO-1-OXO]PROPYL](N-METHYL)- AMINO]BUTOXY]-NAPHTHALENE-2- (N-ACETAMIDO)CARBOXAMIDE | 0.33 |
| 154 | 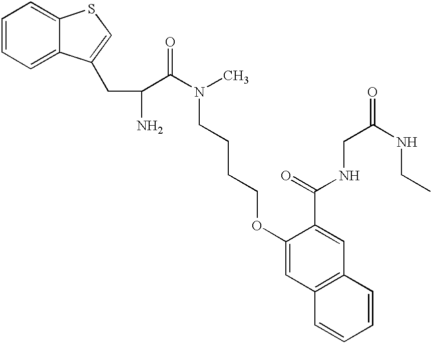 | 3-[[[4-[3-(BENZOTHIOPHEN-3-YL)-2- AMINO-1-OXO]PROPYL](N-METHYL)- AMINO]BUTOXY]-NAPHTHALENE-2- [(N-(N-ETHYL)ACETAMIDO]- CARBOXAMIDE | 4 |
| 155 | 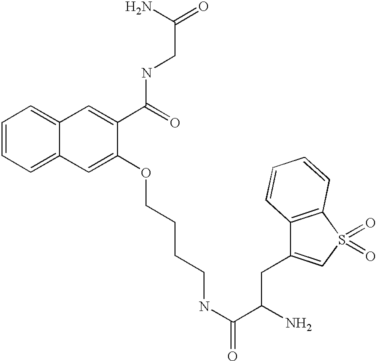 | 3-[[4-[N-[1-OXO-2(S)-(AMINO)-3-(1,1- DIOXOBENZOTHIOPHEN-3-YL)]- PROPYL]AMINO] BUTOXY]- NAPHTHALENE-2- (N-ACETAMIDO)CARBOXAMIDE | 5 |
| 156 | 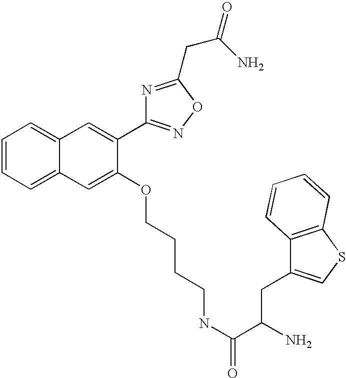 | 2-(5-ACETAMIDO-1,2,4-OXADIAZOL-3- YL)-3-[[4-[N-[1-OXO-2(S)-(AMINO)-3- (BENZOTHIOPHEN-3-YL)]PROPYL]- AMINO]BUTOXY]-NAPHTHALENE | 20 |

Formula I includes compounds 5-12, and 14;

Formula II includes compounds 13, 15-19, 31 and 36-51;

Formula III includes compounds 1, 20-25, 29, 33-34 and 86;

Formula IV includes compounds 26-28, 30 and 71;

Formula V includes compounds 107-111 and 114-118;

Formula VI includes compounds 2-4;

Formula VII includes compounds 151, 152 and 156;

Formula VIII includes compounds 119-127, 129-130, 132-135, 137-140, 143, 147-150 and 155;

Formula IX includes compounds 128, 131, 141 and 142;

Formula X includes compounds 80-85 and 87;

Formula XI includes compounds 88-96 and 99-104;

Formula XII includes compounds 97 and 98;

Formula XIII includes compounds 105, 106, 153 and 154;

Formula XIV includes compounds 52-70 and 72-74;

Formula XV includes compounds 75-79;

Formula XVI includes compounds 32 and 35;

Formula XVII includes compounds 144-146; and

Formula XVIII includes compounds 112-113 and 136.

Compounds included in Formula I, II, III, V, VIII, X, XI, XIII, XIV, XV XVI and XVIII are prepared by the process set forth in scheme A. Compounds included in Formula IV and XV are prepared according to Scheme B. Compounds included in formula XIV are prepared according to the process set forth in Scheme C. Compounds included in Formula XI are prepared according to the process set forth in Scheme D. Compounds included in Formula XIV are prepared according to the process set forth in Scheme E or Scheme F. Compounds prepared according to Schemes Q and R are included in Formula XIII. The compounds set forth hereinabove have been prepared according to the following breakdown.

SCHEME A

Compound Nos. 5, 6, 8, 9, 10, 11, 12, 13, 14, 15, 16, 17, 18, 19, 20, 21, 22, 23, 24, 25, 26, 29, 31, 32, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 70, 74, 78, 79, 92, 93, 94, 95, 96, 97, 98, 99, 100, 101, 102, 103, 105, 106, 107, 108, 109, 110, 111, 112, 113, 114, 115, 116, 117, 118, 122, 125, 126, 136, 143

SCHEME B

Compound Nos. 27, 28, 30, 75, 76, 77

SCHEME C

Compound Nos. 72, 73

SCHEME D

Compound No. 104

SCHEME E

Compound Nos. 68, 69

SCHEME F

Compound No. 71

SCHEME G

Compound Nos. 1, 2, 3, 4, 7

SCHEME H

Compound No. 119, 120, 121

SCHEME I

Compound No. 124

SCHEME J

Compound No. 129, 130

SCHEME K

Compound Nos. 128, 131

SCHEME L

Compound No. 134, 135

SCHEME M

Compound Nos. 144, 145, 146

SCHEME N

Compound Nos. 151, 152

SCHEME O

Compound No. 155

SCHEME P

Compound No. 156

SCHEME Q

Compound Nos. 153

SCHEME R

Compound Nos. 154

SCHEME S

Compound Nos. 80, 81, 82, 83, 84, 85, 86, 87 and 123.

SCHEME T

Compound Nos. 33, 34, 35, 88, 89, 90, 91

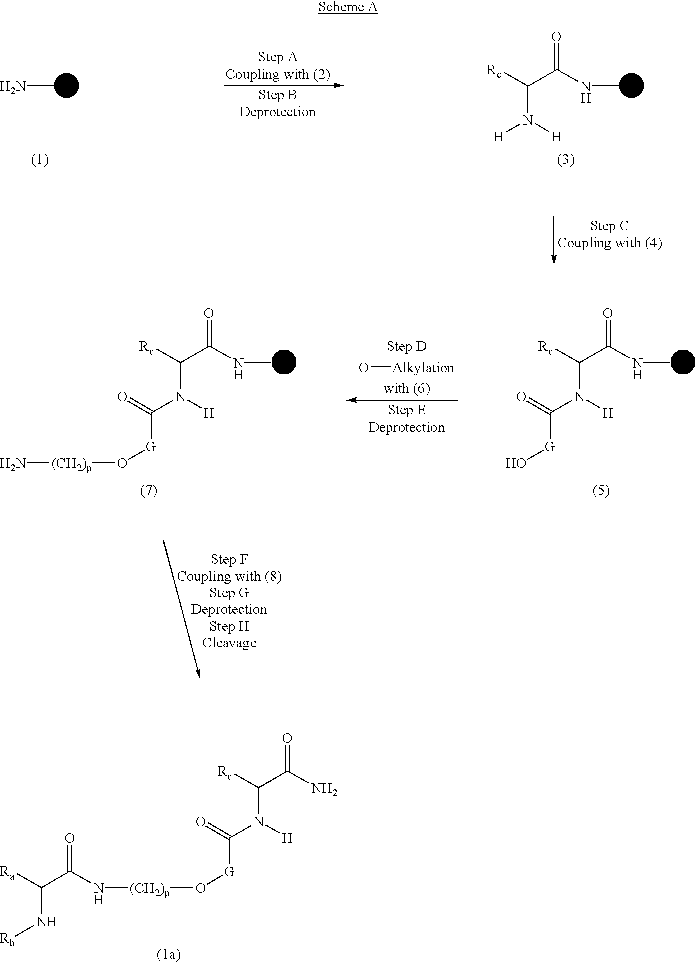

In Scheme A, step A, compound (2)

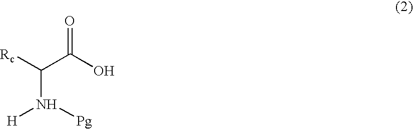

is coupled to a solid phase support (1) using standard peptide synthesis techniques well known in the art to provide an amide linkage, the compound (2). Such coupling can be carried out using standard coupling procedures known by those of ordinary skill in the art as described in Stewart and Young, “Solid Phase Peptide Synthesis”, 2nd ed., Pierce Chemical Co., Rockford, Ill. (1984); Gross, Meienhofer, Udenfriend, Eds., “The Peptides: Analysis, Synthesis, Biology”, Vol 1, 2, 3, 5 and 9, Academic Press, New York, 1980-I 987; Bodanszky, “Peptide Chemistry: A Practical Textbook,” Springer-Verlag, New York (1988); and Bodanszky, et al. “The Practice of Peptide Synthesis” Springer-Verlag, New York (1984)’ the disclosures of which are hereby incorporated by reference. Particularly preferred are the azide method, the symmetrical and mixed anhydride method, active esters generated in situ or preformed, and the formation of amide linkages with the aid of coupling reagents (activators), especially dicyclohexylcarbodiimide (DCC), diisopropylcarbodiimide (DIC), 1-ethoxycarbonyl-2-ethoxy-1,2-dihydroquinoline (EEDQ), 1-ethyl-3-(3-dimethylaminopropyl)-carbodiimide hydrochloride (EDCI), n-propanephosphonic anhydride (PPA), N,N-bis(2-oxo-3oxazolidinyl)amidophosphoryl chloride (BOP-Cl), diphenylphosphoryl azide, (DPPA), Castro's reagent (BOP), 2-(1 H-benzotriazol-1-yl)-1,1,3,3-tetramethyluronium salts (HBTU), 2,5-diphenyl-2,3-dihydro-3-oxo-4-hydroxy-thiophene dioxide (Steglich's reagent; HOTDO) and 1,1′-carbonyldiimidazole (CDI). The coupling reagents can be used alone or combined with additives such as 4-dimethylaminopyridine (DMAP), N-hydroxybenzotriazole (HOBt), N-hydroxybenzotriazine (HOOBt), N-hydroxy-succinimide (HOSu) or 2-hydroxypyridine. These coupling reactions can be performed in either solution (liquid phase) or solid phase.

Examples of protecting groups (Pg) on structure (2) include the following: 1) acyl types such as formyl, trifluoroacetyl, phthalyl, and p-toluenesulfonyl; 2) aromatic carbamate types such as benzyloxycarbonyl (Cbz or Z) and substituted benzyloxycarbonyls, 1-(p-biphenyl)-1-methylethoxy-carbonyl, and 9-fluorenylmethyloxycarbonyl (Fmoc); 3) aliphatic carbamate types such as tert-butyloxycarbonyl (Boc), ethoxycarbonyl, diisopropyl-methoxycarbonyl, and allyloxycarbonyl; 4) cyclic alkyl carbamate types such as cyclopentyloxycarbonyl and adamantyloxycarbonyl; 5) alkyl types such as triphenyl-methyl and benzyl; 6) trialkylsilane such as trimethylsilane; and 7) thiol containing types such as phenylthio-carbonyl and dithiasuccinoyl. The preferred protecting group is either Boc or Fmoc.

One of ordinary skill in the art readily appreciates that certain functional groups at R_{c} on compound (2) must also be protected during the coupling reactions to avoid formation of undesired bonds. The protecting groups that can be used are listed in Greene, “Protective Groups in Organic Chemistry”, John Wiley & Sons, New York (1981) and “The Peptides: Analysis, Synthesis, Biology”, Vol. 3, Academic Press, New York (1981)’ the disclosure of which is hereby incorporated by reference. Those skilled in the art will appreciate that the selection and use of appropriate protecting groups on R_{c} depends upon the overall structure of compound (2) and the presence of other protecting groups on compound (2). The selection of such a protecting group is important in that it must not be removed during the deprotection in step B wherein Pg is removed.

For example, when Boc is used as the protecting group (Pg), the following side chain protecting groups on R, are suitable: p-toluenesulfonyl (tosyl) moieties can be used to protect the amino side chains of amino acids such as Lys and Arc; p-methylbenzyl, acetamidomethyl, benzyl (Bzl), or t-butylsulfonyl moieties can be used to protect the sulfide containing side chains of amino acids such as cysteine; and benzyl (Bzl) ether can be used to protect the hydroxy containing side chains of amino acids such as Ser or Thr.

When Fmoc is chosen for the protecting group (Pg), usually tert-butyl based protecting groups on R_{c} are acceptable. For instance, Boc can be used for lysine, tert-butyl ether for serine and threonine, and tert-butyl ester for glutamic acid.

More specifically, a solid phase support (I), such as a deprotected Rink resin is treated with about 3 equivalents of compound (2) and about 3 equivalents of 1-hydroxybenzotriazole in a suitable organic solvent, such as N,N-dimethylformamide. Examples of compound (2) include N-Fmoc-glycine, N-Fmoc-L-alanine, N-Fmoc-D-alanine, N-Fmoc-beta alanine, N-Fmoc-norleucine, N-Fmoc-arginine, N-Fmoc-N-methylglycine, N-Fmoc-L-tryptophan, N-Fmoc-L-phenylalanine and the like.

Then about 3 equivalents of diisopropylcarbodiimide are added and the mixture is shaken for about 30 minutes to five hours. The amide that is produced can be isolated and purified by techniques well known to one of ordinary skill in the art or the crude material can be carried on tot step B for deprotection.

In Scheme A, step B, the amide produced in step A is deprotected under conditions well known in the art which do not cleave the solid phase support from the growing compound. When the Boc protecting group is used, the methods of choice are trifluoroacetic acid, neat or in dichloromethane, or HCI in dioxane or ethyl acetate. The resulting ammonium salt is then neutralized either prior to the coupling or in situ with basic solutions such as aqueous buffers, or tertiary amines in dichloromethane or dimethylformamide. When the Fmoc group is used, the reagents of choice are piperidine or substituted piperidine in dimethylformamide, but any secondary amine or aqueous basic solutions can be used. The deprotection is carried out at a temperature of between about 0° C. and about room temperature.

For example, the crude material produced in step A, wherein Pg is an Fmoc protecting group, is treated with 30% piperidine in N,N-dimethylformamide for about 20 minutes to about one hour. The reaction mixture is filtered and rinsed with a suitable organic solvent to provide the deprotected compound (3).

In Scheme A, step C, the compound (3) is coupled to compound (4)

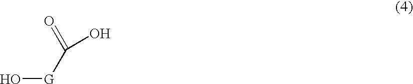

in a manner analogous to the procedure described in step A above to provide the compound (5). For example, about 3 equivalents of compound (4) is combined with compound (3) and about 3 equivalents of 1-hydroxybenzotriazole in a suitable organic solvent, such as N,N-dimethylformamide. Examples of compound (4) are 3-hydroxy-2-naphthoic acid, 2-hydroxy-3-dibenzofuran carboxylic acid, salicylic acid, 3-hydroxy-4,7-dibromonaphthoic acid, 7-methoxy-3-hydroxy-2-napthoic acid, 1-hydroxy-4-bromo-2-naphthoic acid, 5iodosalicylic acid, 3-hydroxy-pyridine-2-carboxylic acid, 2-hydroxy-1-naphthoic acid, 3,4,5-trihydroxybenzoic acid, 1-phenyl-4-hydroxy-2-naphthoic acid, 6-hydroxy-2-naphthoic acid, and the like. Then about 3 equivalents of diisopropylcarbodiimide are added and the mixture is shaken for 30 minutes to 5 hours. The resin is then washed with suitable organic solvents, such as N,N-dimethylformamide and anhydrous tetrahydrofuran to provide the compound (5).

In Scheme A, step D, compound (5) is alkylated under conditions well known in the art with compound (6)

Pg²—NH—(CH₂)ₚ—D (6)

wherein D is —OH, Cl, Br or I, p is an integer from 2 to 6 and Pg² is a suitable protecting group chosen in a manner analogous to Pg, such as Fmoc or Boc, followed by deprotection in step E to provide the compound (7). For example, a solution of 6 equivalents of triphenylphosphine and an excess of compound (6), wherein D is —OH, in a suitable organic solvent, such as tetrahydrofuran are added to compound (5). Examples of compound (6) are N-Fmoc-aminobutanol and the like. The mixture is cooled to about −15° C. for 15 minutes and about 6 equivalents of diisopropylazodicarboxylate dissolved in a suitable organic solvent, such as tetrahydrofuran is added to the cooled mixture. The mixture is then shaken for about 6 to 12 hours and then filtered. The resin is washed with suitable organic solvents, such as N,N-dimethylformamide, methylene chloride, tetrahydrofuran and then N,N-dimethylformamide. In step E, the protecting group is removed under conditions well known in the art. For example, when the protecting group (Pg²) is an Fmoc protecting group, the compound produced in step D is treated with 30% piperidine in N,N-dimethylformamide for about 20 minutes. The resin is then washed with suitable organic solvents, such as N,N-dimethylformamide and 5% 1-hydroxybenzotriazole in N,N-dimethylformamide to provide compound (7).

In Scheme A, step F, compound (7) is coupled with compound (8)

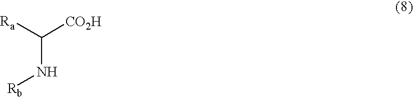

in a manner analogous to the procedure described in step A above followed by deprotection in step G and cleavage from the resin in step H to provide the compound of formula (1a). For example, compound (7) is combined with about 3 equivalents of compound (8) and about 3 equivalents of I-hydroxybenzotriazole in a suitable organic solvent, such as N,N-dimethylformamide. Examples of compound (8) are Fmoc-L-3-(3-benzothienyl)alanine, Fmoc-L-tyrosine, Fmoc-L-3-pyridylalanine, Fmoc-L-4-pyridylalanine, Fmoc-L-cyclohexylalanine, Fmoc-L-cyclohexylglycine, Fmoc-L3-nitrotyrosine, Fmoc-L-2-indanylglycine, Fmoc-D-phenylalanine, Fmoc-D-tyrosine, Fmoc-L-2-thienylalanine, Fmoc-L-4-thiazolylalanine, Fmoc-L-tryptophan, Fmoc-L-3-fluorophenylalanine, Fmoc-L-4-fluorophenylalanine, Fmoc-L-1-naphthylalanine, Fmoc-L-2-naphthylalanine, Fmoc-L-3-iodotyrosine, Fmoc-L-4-chlorophenylalanine, Fmoc-L-3,4-dichlorophenylalanine, Fmoc-L-4-nitrophenylalanine, Fmoc-L-4-bromophenylalanine, Fmoc-L-4-iodophenylalanine, Fmoc-L-4-aminophenylalanine, Fmoc-L-pentafluorophenylalanine, Fmoc-L-homophenylalanine, Fmoc-L-phenylglycine, Fmoc-L-phenylalanine, Fmoc-L-diphenylalanine, Fmoc-L-4,4-biphenylalanine, Fmoc-L-S-benzylcystine, Fmoc-L-leucine, Fmoc-glycine, Fmoc-L-alanine, Fmoc-t-butylglycine, Fmoc-L-N-methylphenylalanine, Fmoc-glutamine, Fmoc-leucine, CBZ-tyrosine, Fmoc-N-benzoyltyrosine, and the like. To this mixture is added about 3 equivalents of diisopropylcarbodiimide and the mixture is shaken for about 30 minutes to about 5 hours. The mixture is then filtered and the resin is washed with suitable organic solvents, such as N,N-dimethylformamide or methylene chloride to provide the amide.

In step G, the protecting group Pg² is removed under conditions well known in the art, in a manner analogous to the procedure described in step B above. In step H, the compound is cleaved from the solid phase support under conditions well known in the art. For example, the cleavage reactions most commonly used are acid- and palladium-catalyzed, especially cleavage in liquid anhydrous hydrogen fluoride, in anhydrous trifluoromethanesulfonic acid, in dilute or concentrated trifluoroacetic acid or palladium-catalyzed cleavage in tetrahydrofuran or tetrahydrofuran-methylene chloride mixtures in the presence of a weak base such as morpholine.

In addition, steps G and H can occur simultaneously wherein the protecting group Pg² is removed and the compound is cleaved from the resin. For example, wherein Pg² is an Fmoc protecting group, the amide is treated with 95:5 trifluoroacetic acid:water for about 45 minutes and the solvents are then removed under vacuum. The crude product is isolated and purified by techniques well known to one of ordinary skill in the art. For example, the residue is triturated with a suitable organic solvent, such as diethyl ether and collected by filtration. The collected material is then purified by chromatography, such as HPLC utilizing a suitable eluent, such as acetonitrile/water to provide the purified compound of Formulae I, II, III, V, VIII, X, XI, XIII, XIV, XV XVI and XVIII, including the compounds listed in Table 1.

In an analogous manner, the compounds as listed above were prepared in accordance with the steps as set forth in the following Schemes B through F. As it is apparent, in most of these instances similar chemical reactions were carried out except for the starting resin as indicated and the reactants.

### Schemes B through F:

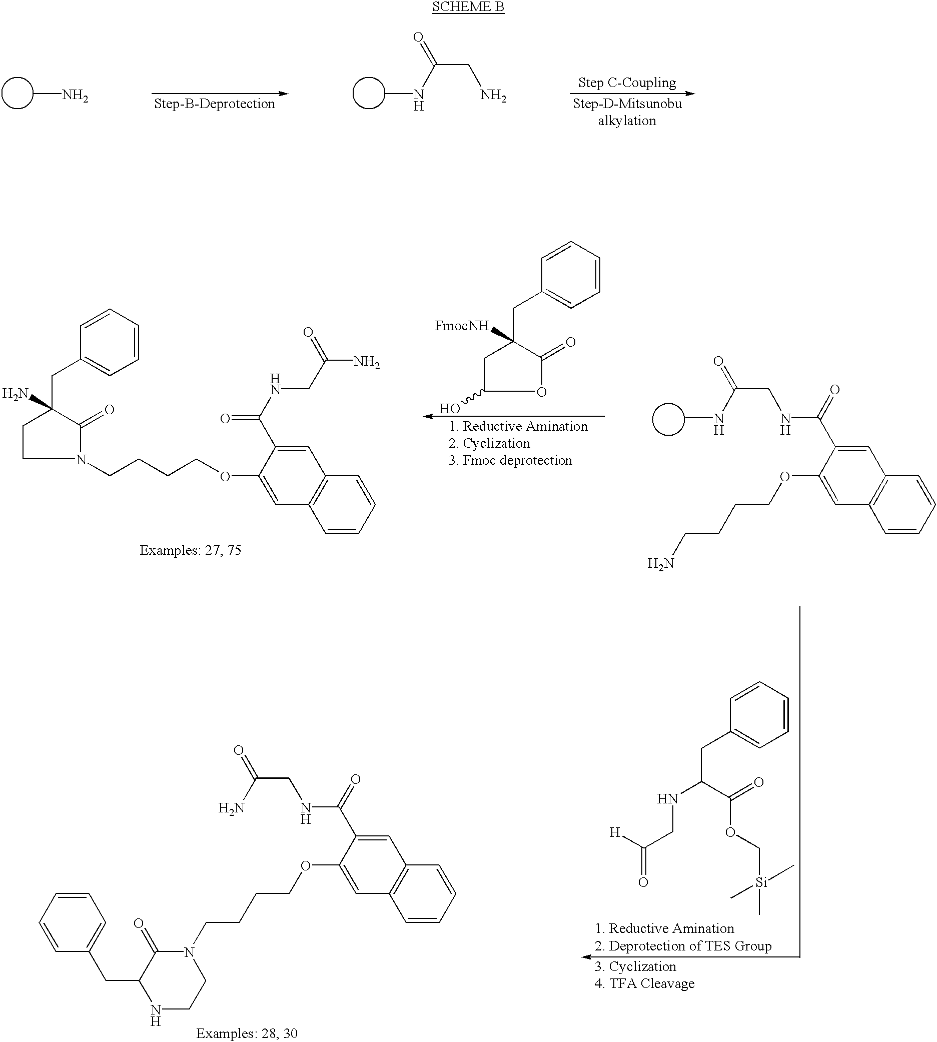

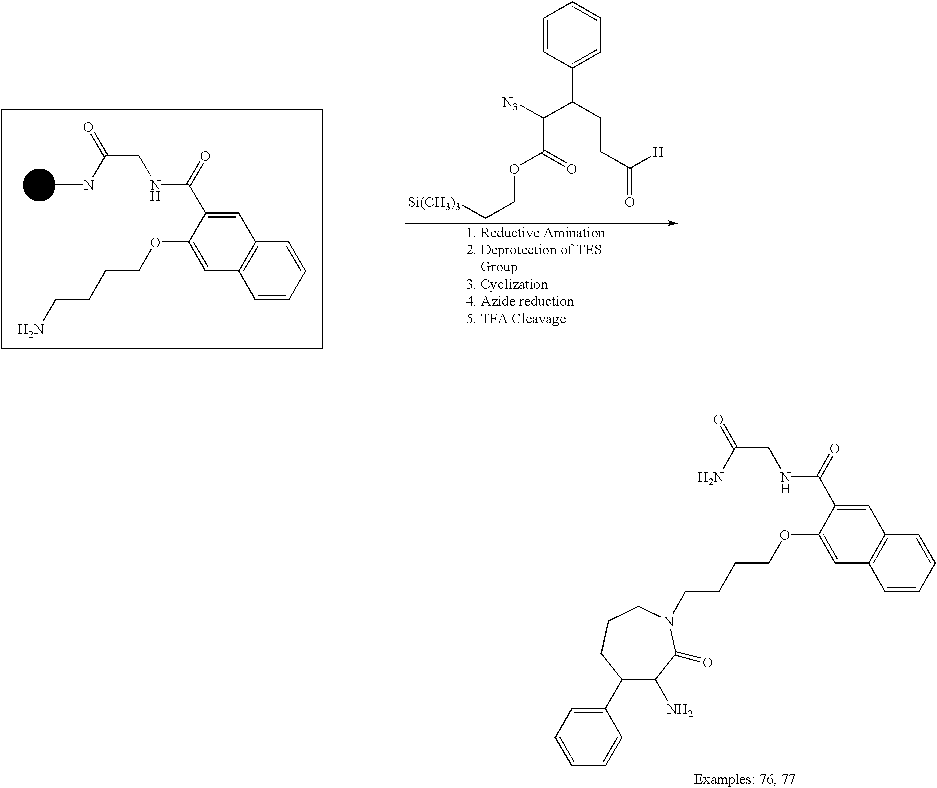

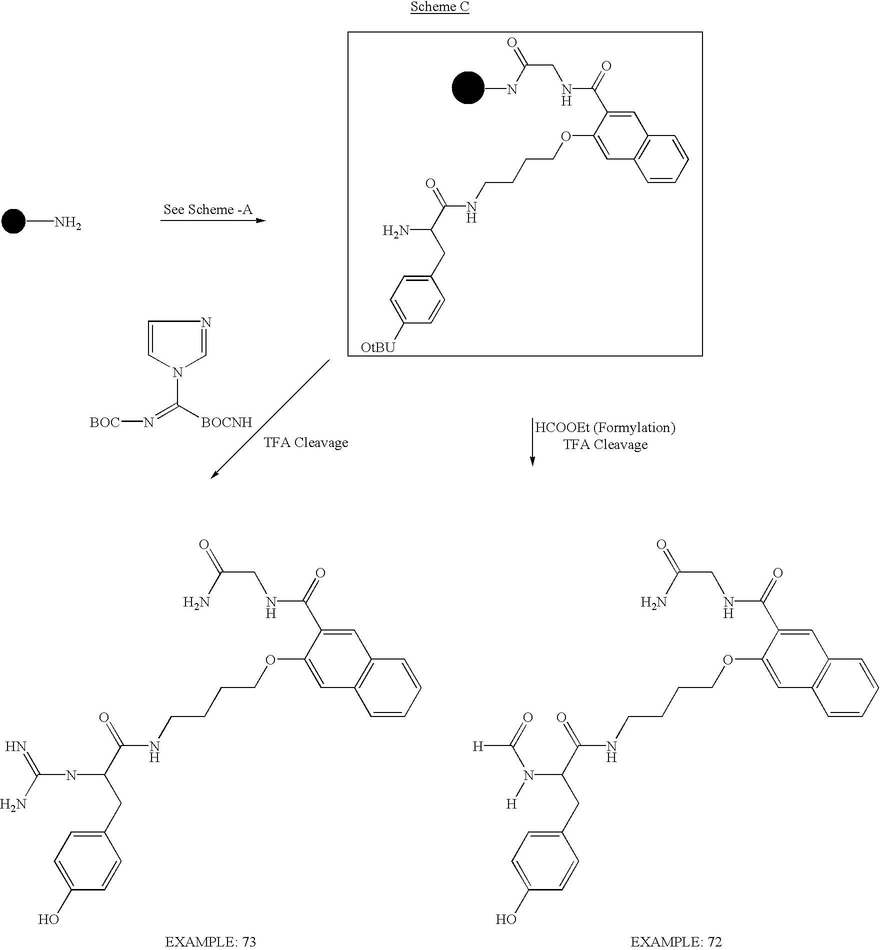

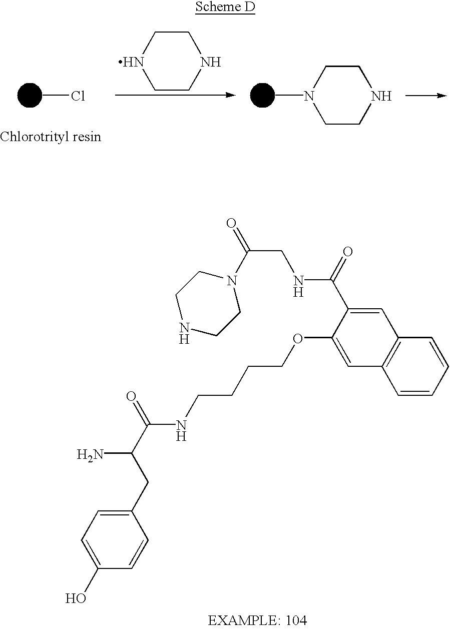

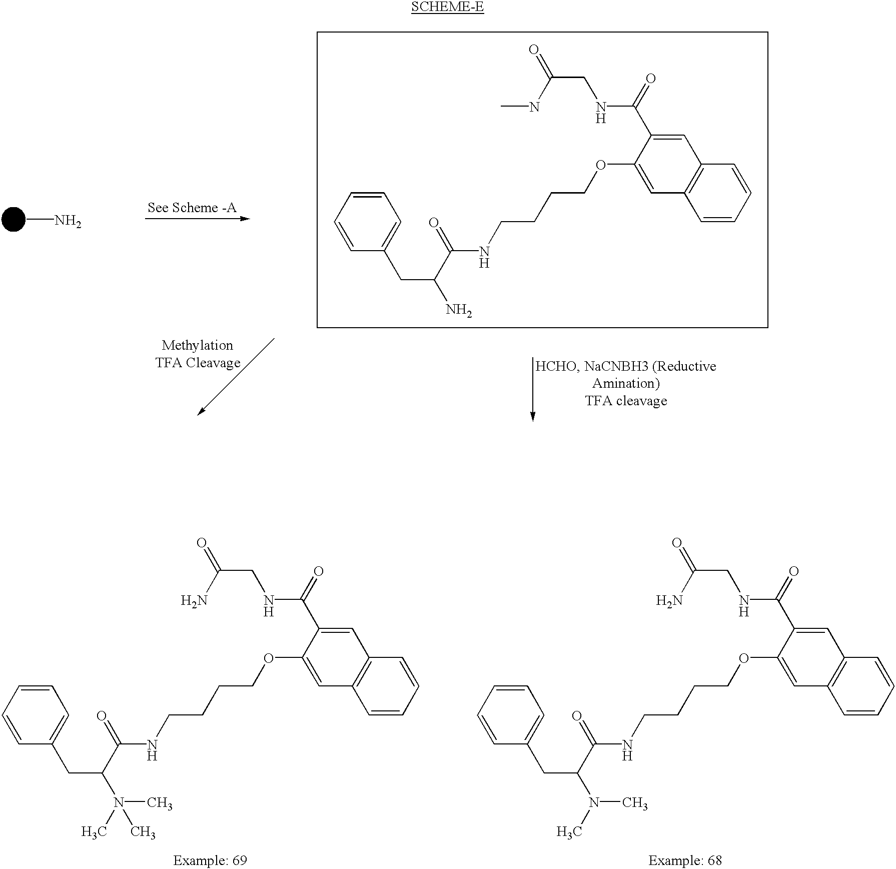

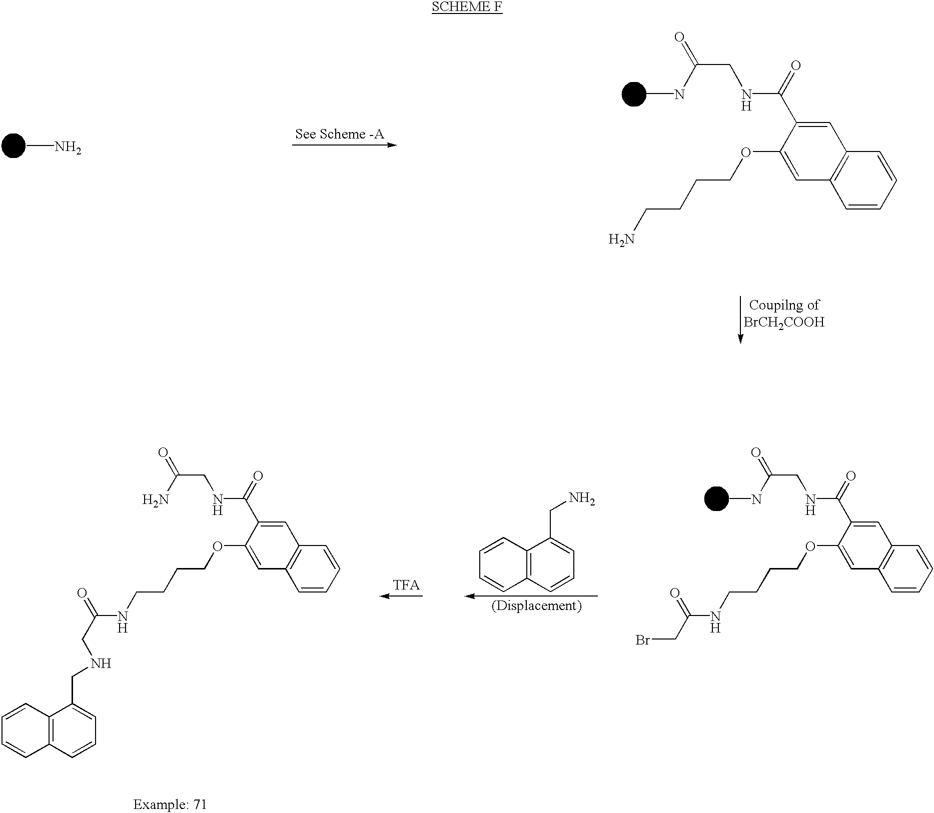

In Scheme G, step A, Compound (10)

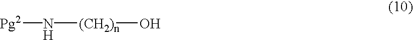

is coupled to a solid phase support HO-SS and deprotected in a manner analogous to the procedure performed in Scheme A, steps D and E, wherein n is 3 or 4, and Pg² is as previously disclosed. Examples of compound (10) are N-Fmoc-aminopropanol, N-Fmoc-aminobutanol and the like.

In Scheme G, step B, the compound (11) is first coupled with N-Fmoc-Glycine, deprotected and then coupled with compound (4)

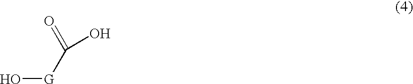

wherein G is phenyl or naphthyl moiety, in a manner analogous to the procedure described in Scheme A, step C to provide the compound (12).

In Scheme G, step C, compound (12) is alkylated with compound (6)

Pg²—NH—(CH₂)ₚ—D (6)

wherein D, p, and Pg² are as described previously, in a manner analogous to that described in Scheme A, step D. This is followed by step D, which involves deprotection in a manner similar to that described in Scheme A, step E to provide the compound (13).

In Scheme G, step E, compound (13) is coupled with compound (8)

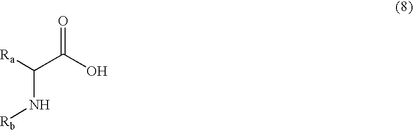

in a manner analogous to the procedure described in Scheme A, step F followed by deprotection in step F and cleavage from the resin in step G to provide the compound of formula (1c). The deprotection reaction of step F is conducted under conditions well known in the art and in a manner analogous to the procedures described in Scheme A, step B. The deprotected product is then cleaved from the solid phase support under conditions well known in the art, in a manner analogous to the procedure described in Scheme A, step H. The deprotection and cleavage steps can also occur simultaneously under reaction conditions similar to those described in Scheme A. The crude product is isolated and purified by techniques well known to one of ordinary skill in the art and as described in Scheme A to provide a compound of Formulae I, III and VI and particularly compounds 1-4 and 7.

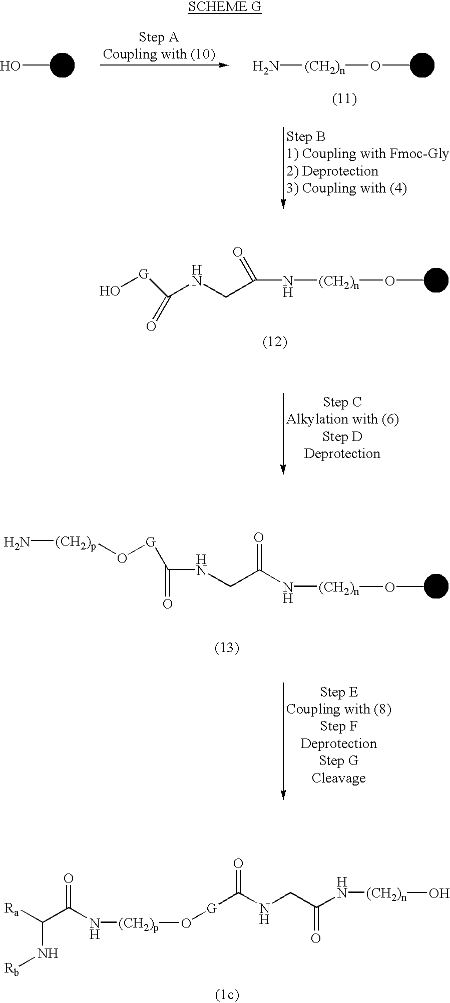

Isolation of the compound at various stages of the reaction scheme may be achieved after cleavage from solid support by standard techniques such as, for example, filtration, evaporation of solvent and the like. Purification of the product, intermediate and the like, may also be performed by standard techniques such as recrystallization, distillation, sublimation, chromatography, conversion to a suitable derivative which may be recrystallized and converted back to the starting compound, and the like. Such techniques are well known to those skilled in the art.

The thus prepared compounds may be analyzed for their composition and purity as well as characterized by standard analytical techniques such as, for example, elemental analysis, NMR, mass spectroscopy, and IR spectra.

In another embodiment, this invention provides pharmaceutical compositions comprising the above-described inventive compounds as an active ingredient. The pharmaceutical compositions generally additionally comprise a pharmaceutically acceptable carrier diluent, excipient or carrier (collectively referred to herein as carrier materials). Because of their therapeutic activity against osteoporosis and bone tissue loss, such pharmaceutical compositions possess utility in treating those diseases.

In yet another embodiment, the present invention discloses methods for preparing pharmaceutical compositions comprising the compounds of Formula I or Formula II as an active ingredient. In the pharmaceutical compositions and methods of the present invention, the active ingredient or ingredients will generally be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like.

Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. antihistaminic activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions As an example may be mentioned water or water-propylene glycol solutions for parenteral injections or addition of sweeteners and pacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active components, e.g., an effective amount to achieve the desired purpose.

The quantity of the inventive active composition in a unit dose of preparation may be generally varied or adjusted from about 1.0 milligram to about 1,000 milligrams, preferably from about 1.0 to about 950 milligrams, more preferably from about 1.0 to about 500 milligrams, and typically from about 1 to about 250 milligrams, according to the particular application. The actual dosage employed may be varied depending upon the patient's age, sex, weight and severity of the condition being treated. Such techniques are well known to those skilled in the art.

Generally, the human oral dosage form containing the active ingredients can be administered 1 or 2 times per day. The amount and frequency of the administration will be regulated according to the judgment of the attending clinician. A generally recommended daily dosage regimen for oral administration may range from about 1.0 milligram to about 1,000 milligrams per day, in single or divided doses.

The term “capsule” refers to a special container or enclosure made of methylcellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

The term “tablet” refers to a compressed or molded solid dosage form containing the active ingredients with suitable diluents. The tablet can be prepared by compression of mixtures or granulations obtained by wet granulation, dry granulation or by compaction.

The term “oral gel” refers to the active ingredients dispersed or solubilized in a hydrophillic semi-solid matrix.

The term “powders for constitution” refers to powder blends containing the active ingredients and suitable diluents which can be suspended in water or juices.

The term “diluent” refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

The term “disintegrant” refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

The term “binder” refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

The term “lubricant” refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d,1-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

The term “glident” refers to those materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

The term “coloring agent” refers to excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

The term “bioavailability” refers to the rate and extent to which the active drug ingredient or therapeutic moiety is absorbed into the systemic circulation from an administered dosage form as compared to a standard or control.

Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

Another embodiment of the invention discloses use of the pharmaceutical compositions disclosed above for treatment of diseases such as, for example, osteoporosis and bone tissue loss.

It will be apparent to those skilled in the art that many modifications, variations and alterations to the present disclosure, both to materials and methods, may be practiced. Such modifications, variations and alterations are intended to be within the spirit and scope of the present invention.

The following examples are being provided to further illustrate the present invention. They are for illustrative purposes only; the scope of the invention is not to be considered limited in any way thereby.

### EXAMPLES

Unless otherwise stated, the following abbreviations have the stated meanings in the Examples below:

DCC=dicyclohexylcarbodiimide

NaBH(OAc)₃=sodium triacetoxyborohydride

FMOC=9-fluorenylmethyloxycarbonyl

DCE=1,2-dichloroethane

DIEA=diisopropylethylamine

Cha=cyclohexylalanine

Nal(1)=1-naphthylalanine

TEOF=triethylorthoformate

TIPS=triisopropylsilane

Nal(1)=1-naphthylalanine

Bip=4-biphenylalanine

Boc-tert.-butyloxycarbonyl

Pip=piperidine

HOAc=acetic acid

TFA=trifluoroacetic acid

Py=pyridine

DIC=diisopropylcarbodiimide

MeOH=methanol

NaBH₄=sodium borohydride

NaBH₃CN=sodium cyanoborohydride

p-TsOH=p-toluenesulfonic acid

DMF: N,N-Dimethylformamide

THF: Tetrahydrofuran

DMSO: Dimethyl sulfoxide

DCM: Dichloromethane which can also be referred to as methylene chloride

LAH: Lithium aluminum hydride

HOAt: 1-Hydroxy-7-azabenzotriazole

HOBt: 1-Hydroxybenzotriazole

HRMS=High Resolution Mass Spectrometry

HPLC=High Performance Liquid Chromatography

NMR=nuclear magnetic resonance

LRMS=Low Resolution Mass Spectrometry

Additionally, “kg” refers to kilograms; “g” refers to grams; “mg” refers to milligrams; μg” refers to micrograms; “m²/g” refers to square meters per gram and is used as a measurement of particle surface area; “mmol” refers to millimoles; “L” refers to liters; “mL” refers to milliliters; “μL” refers to microliters; “cm” refers to centimeters; “M” refers to molar’ “mM” refers to millimolar; “μM” refers to micromolar; “nM” refers to nanomolar; “N” refers to normal; “ppm” refers to parts per million; “δ” refers to parts per million down field from tetramethylsilane; “° C.” refers to degrees Celsius; “° F.” refers to degrees Fahrenheit; “mm Hg” refers to millimeters of mercury; “kPa” refers to kilopascals; “psi” refers to pounds per square inch; “rpm” refers to revolutions per minute; “bp” refers to boiling point; “mp” refers to melting point; “dec” refers to decomposition; “h” refers to hours; “min” refers to minutes; “sec” refers to seconds’ “R_{f}” refers to retention factor; and “Rₜ” refers to retention time.

Phosphorylation Assay of Src Kinase Inhibitors

The activity of Src kinase was measured in a 96 well plate phosphorylation assay where the transfer of gamma phosphate from ATP to peptide substrate was measured radiometrically. The percentage inhibition and IC₅₀ values were calculated from duplicates of samples tested in the range of 400-0.098 M (2 or 4 fold dilutions). Kᵢ values were determined experimentally for some of the inhibitors. The phosphocellulose filter plates (Millipore catalog #MAPH NOB) were prewet prior to the phosphorylation assay by addition of 100 1 per well of 30 mM MES buffer+0.4 mg/ml BSA. After a 20-minute incubation, the buffer was removed completely via a Millipore vacuum manifold. The phosphorylation assay was then set up in the filter plates in a total volume per well of 60 1. The 60 1 consisted of compound (see concentration ranges above), a 1:100 dilution of Src kinase (originally Upstate Bioteolnology, #14-117; later HMR stock 27597-019E1) or assay buffer alone (for no enzyme control wells), 10 M peptide substrate (FIYGAFKKKKNH₂), 5 M unlabeled ATP and a 1:500 dilution of P-ATP (New England Nuclear, catalog #NEG-602H, 2000 Ci/mmol). The reaction was mixed and incubated at room temperature for 30 min. The kinase reactions were stopped by addition of 150 mM H₃PO₄ and incubated for 10 minutes. The filter plate wells were filtered and washed seven times with 200 1 per well of 150 mM H₃PO₄. Finally, the filter plate was placed into a Packard plate adapter (Packard catalog #600517B), 35 1 of Microscint-20 (Packard catalog #6013621) was added to each well, the plate was sealed (TopSeal A sealers; Packard catalog #6005185) and counted on a Packard TopCount.

General Synthesis Procedures

Starting materials used in the synthesis were obtained from chemical vendors such as Aldrich, Sigma, Fluka, Nova Biochem and Advanced Chemtech. During the synthesis, the functional groups of the amino acid derivatives used were protected by blocking groups to prevent side reaction during the coupling steps. Examples of suitable protecting groups and their use are described in The Peptides, supra, 1981, and in vol. 9, Udenfriend and Meienhofer (eds.), 1987, which is incorporated herein by reference.

General solid-phase peptide synthesis was used to produce the compounds of the invention. Such methods are described, for example, by Steward and Young, Solid Phase Peptide Synthesis (Freeman & Co., San Francisco, 1969), which is incorporated herein by reference.

Unless indicated otherwise, peptides were synthesized on RAM™ Polystyrene Resin (Rapp Polymere, Tüibingen, Germany). As an alternative to this, acid sensitive linker p-[(R,S)-α-[1-(9H-fluoren-9-yl)methoxyformamido]-2,4-dimethoxybenzyl]phenoxyacetic acid (Knorr Linker, Bernatowicz et. al, Tetr. Lett. 30 (1989) 4645, which is incorporated herein by reference) can be coupled to any amino functionalized solid support or the desired compounds can be synthesized on polystyrene resin cross-linked with 1% divinylbenzene modified with an acid sensitive linker (Rink resin) (Rink, Tetr. Lett. 28 (1987) 3787; Sieber, Tetr. Lett. 28 (1987) 2107, each of which is incorporated herein by reference). Coupling was performed using N,N′-diisopropylcarbodiimide (DIC) in the presence of an equivalent amount of HOBt. All couplings were done N,N-dimethylformamide (DMF) at room temperature (RT). Completion of coupling was monitored by ninhydrin test. A second (double) coupling was performed where coupling in the first instance was incomplete.

Deprotection of the Fmoc group was accomplished using 50% piperidine in DMF for 2+15 min. The amount of Fmoc released was determined from the absorbance at 302 nm of the solution after deprotection, volume of washes and weight of the resin used in the synthesis.

The compound resin was at the end of the synthesis washed successively with DMF and DCM and the peptide was then cleaved and deprotected by a mixture of TFA/TIPS (99/1) for 2 hours, unless specified otherwise. The resin was washed with DCM and the DCM wash combined with the TFA releasate. The solution was evaporated, the product was redissolved in a mixture of water and acetonitrile and lyophilized.

The dried compound was subjected to HPLC purification using an appropriate gradient of 0.1% TFA in water and acetonitrile (ACN). After collecting the peak containing the intended synthetic product, the solution was lyophilized and the compound was subjected to an identification process, which included electrospray mass spectrum (MS) and/or NMR to confirm that the correct compound was synthesized.

For HPLC analysis, a sample of the product was analyzed using Beckman HPLC system (consisting of 126 Solvent Deliver System, 166 Programmable Detector Module 507e Autosampler, controlled by Data Station with Gold Nouveau software) and YMC ODS-AM 4.6×250 mm column at 230 nm and flow rate 1 ml/min.

For product purification, a sample of crude lyophilized compound was dissolved in a mixture of 0.1% aqueous TFA containing 10% to 50% ACN. The solution of the product was usually filtered through a syringe connected to a 0.45 μm “ACRODISC” 13 CR PTFE (Gelman Sciences; Ann Arbor Mich.) filter. A proper volume of filtered compound solution was injected into a semi-preparative C18 column (YMC ODS-A column (20×250 mm), YMC, Inc., Wilmington, N.C.). The flow rate of a gradient or isocratic mixture of 0.1% TFA buffer and ACN (HPLC grade) as an eluent was maintained using a Beckman “SYSTEM GOLD” HPLC (Beckman, System Gold, Programmable Solvent Module 126 and Programmable Detector Module 166 controlled by “SYSTEM GOLD” software). Elution of the compound was monitored by UV detection at 230 nm. After identifying the peak corresponding to the compound under synthesis using MS, the compound was collected, lyophilized and biologically tested. MS was performed using a VG Platform (Fisons Instruments) instrument in ES+ mode. For NMR, typically samples were measured in DMSO-d₆ (Aldrich) using a Bruker Avance DPX 300 instrument.

### Example I (Sch. A) (Compound 122)

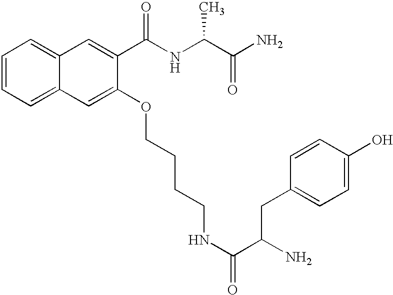

Scheme A, steps A and B; D-Ala Rink resin (200 mg), is washed with DMF (3×30 mL). The Fmoc group is removed by treatment with 30% piperidine in DMF (2×25 mL: 1×5 min; 1×15 min). The resin is washed with DMF (3×50 mL), 5% HOBt in DMF (2×50 mL) and DMF (2×30 mL). A solution of Fmoc tyrosine and HOBt (3 mmoles, 405 mg) in DMF (15 mL) is added to the resin followed by addition of diisopropylcarbodiimide (3 mmol, 471 L). The mixture is then shaken for 1 hour. The Fmoc protecting group is then removed by treatment with 30% piperidine in DMF (2×25 mL; 1×5 min; 1×15 min).

Scheme A, step C: A solution of 3-hydroxy-2-naphthoic acid (180 mg) and HOBt (3 mmoles, 405 mg) in DMF (15 mL) is added to the deprotected compound of step B above, followed by addition of diisopropylcarbodiimde (3 mmoles, 471 L). The mixture is shaken for 30 min. and then filtered. The resin is washed with DMF (5×30 mL) and anhydrous THF.

Scheme A, steps D and E: A solution of triphenylphosphine (6 mmols, 1.5 g) and Fmoc aminobutanol in anhydrous THF (12 mL) are added to the resin from step C above. The mixture was kept for 15 min. at −15° C. and a solution of diisopropylazodicarboxylate (6 mmoles, 2 mL) in THF (3 mL) is added to the mixture. The mixture is shaken for 6 hours and then filtered. The resin is then washed with DMF (6×50 mL), methylene chloride (6×50 mL), THF (6×50 mL) and DMF (6×50 mL). The Fmoc protecting group is then removed by treatment with 30% piperidine in DMF (2×25 mL 1×5 min; 1×15 min.) The resin is washed with DMF (3×50 mL) and a solution of 5% HOBt in DMF.

Scheme A, steps F, G and H: About ⅙ of the above deprotected compound from step D above is combined with a solution of Fmoc D-Ala Rink (200 mg) in DMF (3 mL) containing HOBt (0.64 mmoles, 87 mg). To this mixture is added disopropylcarbodiimide (0.674 mmoles, 100 L) and the mixture is shaken for 2 hours. The mixture is then filtered and the resin is washed with DMF (3×5 mL) and methylene chloride (3×5 mL). The resin is then treated with 95:5 trifluoroacetic acid:water (3 mL) for 45 min. and the solvents are then removed under vacuum. The residue is treated with diethyl ether (5 mL) to provide a solid. The solid is purified by preparative HPLC-MS (30 to 80% acetonitrile/water) to provide the final product (Calculated M⁺+H=493; observed M⁺+H=493).

The following additional compounds as set forth in Table 1 below are prepared in a manner analogous to the procedure described above in Example 1, Scheme A, steps A through H wherein a suitably protected amino acid, instead of D-Ala Rink is used and other building blocks set forth in Table 1.

In addition, several other compounds as set forth in Table 1 below are prepared in a manner analogous to the procedure described above in Example 1, however, using the appropriate Schemes as described below and using the appropriate building blocks as set forth in Table 1. For example, compound 104 is prepared using the building blocks as set forth in Table 1 and in accordance with the procedures as set forth in Scheme D. Compounds 68 and 69 are prepared in accordance with the procedures of Scheme E and using the building blocks as listed in Table 1. Similarly, compounds 80 to 87 and 123 are prepared following the procedures of Scheme S and using the building blocks as set forth in Table 1 for these compounds. Finally, compounds 88 to 91 are prepared in accordance with procedures of Scheme T and using the building blocks as listed in Table 1.

TABLE 1

| Example | Building Blocks |
|---|---|
| 5 | Fmoc Tyrosine, Fmoc 4-amino butanol, 2-hydroxy-3- |
| | naphthoic acid, Fmoc glycine |
| 6 | Fmoc Tyrosine, Fmoc 4-amino butanol, 2-hydroxy-3- |
| | naphthoic acid |
| 8 | Fmoc Tyrosine, Fmoc 4-amino butanol, 2-hydroxy-3- |
| | naphthoic acid, Fmoc lysine |
| 9 | Fmoc Tyrosine, Fmoc 4-amino butanol, 2-hydroxy-3- |
| | naphthoic acid, Fmoc alanine |
| 10 | Fmoc Tyrosine, Fmoc 4-amino butanol, 2-hydroxy-3- |
| | naphthoic acid, Fmoc Phe |
| 11 | Fmoc Tyrosine, Fmoc 4-amino butanol, 2-hydroxy-3- |
| | naphthoic acid, Fmoc arginine |
| 12 | Fmoc Tyrosine, Fmoc 4-amino butanol, 2-hydroxy-3- |
| | naphthoic acid, Fmoc glutamic acid |
| 13 | Fmoc 2-Naphthylalanine, Fmoc 4-amino butanol, 2-hydroxy- |
| | 3-naphthoic acid, Fmoc glycine |
| 14 | Fmoc Phenylalanine, Fmoc 4-amino butanol, 2-hydroxy-3- |
| | naphthoic acid, Fmoc glycine |
| 15 | Fmoc Leucine, Fmoc 4-amino butanol, 2-hydroxy-3- |
| | naphthoic acid, Fmoc glycine |
| 16 | Fmoc Lysine, Fmoc 4-amino butanol, 2-hydroxy-3-naphthoic |
| | acid, Fmoc glycine |
| 17 | N-benzoyl Tyrosine, Fmoc 4-amino butanol, 2-hydroxy-3- |
| | naphthoic acid, Fmoc glycine |
| 18 | CBZ Tyrosine, Fmoc 4-amino butanol, 2-hydroxy-3-naphthoic |
| | acid, Fmoc glycine |
| 19 | Fmoc Isoleucine, Fmoc Tyrosine, Fmoc 4-amino butanol, |
| | 2-hydroxy-3-naphthoic acid, Fmoc glycine |
| 20 | Fmoc Tetrahydroisoquinoline carboxylic acid, Fmoc 4-amino |
| | butanol, 2-hydroxy-3-naphthoic acid, Fmoc glycine |
| 21 | 3(R)-(4-hydroxyphenyl) Fmoc Proline, Fmoc 4-amino |
| | butanol, 2-hydroxy-3-naphthoic acid, Fmoc glycine |
| 22 | 3(S)-(4-hydroxyphenyl) Fmoc Proline, Fmoc 4-amino butanol, |
| | 2-hydroxy-3-naphthoic acid, Fmoc glycine |
| 23 | Fmoc Tyrosine, Fmoc 4-amino ethanol, 2-hydroxy-3- |
| | naphthoic acid, Fmoc glycine |
| 24 | Fmoc Tyrosine, Fmoc 4-amino pentanol, 2-hydroxy-3- |
| | naphthoic acid, Fmoc glycine |
| 25 | Fmoc Tyrosine, Fmoc 4-amino hexanol, 2-hydroxy-3- |
| | naphthoic acid, Fmoc glycine |
| 26 | Fmoc 4-amino butanol, 2-hydroxy-3-naphthoic acid, Fmoc |
| | glycine |
| 31 | Fmoc Tyrosine, Fmoc 4-amino butanol, 2-hydroxy-3- |
| | naphthoic acid, Fmoc sarcosine |
| 36 | Fmoc homophenylalanine, Fmoc 4-amino ethanol, 2-hydroxy- |
| | 3-naphthoic acid, Fmoc glycine |
| 37 | Fmoc Phenyl glycine, Fmoc 4-amino butanol, 2-hydroxy-3- |
| | naphthoic acid, Fmoc glycine |
| 38 | Fmoc Biphenylalanine, Fmoc 4-amino butanol, 2-hydroxy- |
| | 3-naphthoic acid, Fmoc glycine |
| 39 | Fmoc Diphenylalanine, Fmoc 4-amino butanol, 2-hydroxy- |
| | 3-naphthoic acid, Fmoc glycine |
| 40 | Fmoc Amino Indane Carboxylic acid, Fmoc 4-amino butanol, |
| | 2-hydroxy-3-naphthoic acid, Fmoc glycine |
| 41 | Fmoc p-Fluoro-Phenylalanine, Fmoc 4-amino butanol, 2- |
| | hydroxy-3-naphthoic acid, Fmoc glycine |
| 42 | Fmoc p-chloro-Phenylalanine, Fmoc 4-amino butanol, 2- |
| | hydroxy-3-naphthoic acid, Fmoc glycine |
| 43 | Fmoc pentafluoro phenylalanine, Fmoc 4-amino butanol, |
| | 2-hydroxy-3-naphthoic acid, Fmoc glycine |
| 44 | Fmoc Benzothienylalanine, Fmoc 4-amino butanol, 2- |
| | hydroxy-3-naphthoic acid, Fmoc glycine |
| 45 | Fmoc p-Nitrophenylalanine, Fmoc 4-amino butanol, 2- |
| | hydroxy-3-naphthoic acid, Fmoc glycine |
| 46 | Fmoc 4-hydroxy-3-Iodo phenylalanine, Fmoc 4-amino |
| | butanol, 2-hydroxy-3-naphthoic acid, Fmoc glycine |
| 47 | Fmoc 2-Pyridylalanine, Fmoc 4-amino butanol, 2- |
| | hydroxy-3-naphthoic acid, Fmoc glycine |
| 48 | Fmoc 3-pyridylalanine, Fmoc 4-amino butanol, 2- |
| | hydroxy-3-naphthoic acid, Fmoc glycine |
| 49 | Fmoc cyclohexylalanine, Fmoc 4-amino butanol, 2- |
| | hydroxy-3-naphthoic acid, Fmoc glycine |
| 50 | Fmoc cyclohexylglycine, Fmoc 4-amino butanol, 2- |
| | hydroxy-3-naphthoic acid, Fmoc glycine |
| 51 | Fmoc Benzothienylalanine, Fmoc 4-amino butanol, 2- |
| | hydroxy-3-naphthoic acid, Fmoc glycine |
| 52 | Fmoc Indanylglycine, Fmoc Benzothienylalanine, Fmoc 4- |
| | amino butanol, 2-hydroxy-3-naphthoic acid, Fmoc glycine |
| 53 | Fmoc D-Phenylalanine, Fmoc Benzothienylalanine, Fmoc 4- |
| | amino butanol, 2-hydroxy-3-naphthoic acid, Fmoc glycine |
| 54 | Fmoc 4-hydroxy-3-naphthoic acid, Fmoc Benzothienylalanine, |
| | Fmoc 4-amino butanol, 2-hydroxy-3-naphthoic acid, Fmoc |
| | glycine |
| 55 | Fmoc thienylalanine, Fmoc Benzothienylalanine, Fmoc 4- |
| | amino butanol, 2-hydroxy-3-naphthoic acid, Fmoc glycine |
| 56 | Fmoc-D-Tyrosine, Fmoc Benzothienylalanine, Fmoc 4-amino |
| | butanol, 2-hydroxy-3-naphthoic acid, Fmoc glycine |
| 57 | Fmoc-1-naphthylalanine, Fmoc Benzothienylalanine, Fmoc 4- |
| | amino butanol, 2-hydroxy-3-naphthoic acid, Fmoc glycine |
| 58 | Fmoc Thiazolylalanine, Fmoc Benzothienylalanine, Fmoc 4- |
| | amino butanol, 2-hydroxy-3-naphthoic acid, Fmoc glycine |
| 59 | Fmoc m-fluoroalanine, Fmoc Benzothienylalanine, Fmoc 4- |
| | amino butanol, 2-hydroxy-3-naphthoic acid, Fmoc glycine |
| 60 | Fmoc Dichlorophenylalanine, Fmoc Benzothienylalanine, |
| | Fmoc 4-amino butanol, 2-hydroxy-3-naphthoic acid, Fmoc |
| | glycine |
| 61 | Fmoc p-Bromophenylalanine, Fmoc Benzothienylalanine, |
| | Fmoc 4-amino butanol, 2-hydroxy-3-naphthoic acid, Fmoc |
| | glycine |
| 62 | Fmoc p-Iodophenylalanine, Fmoc Benzothienylalanine, Fmoc |
| | 4-amino butanol, 2-hydroxy-3-naphthoic acid, Fmoc glycine |
| 63 | Fmoc S-Benzylcysteine, Fmoc Benzothienylalanine, Fmoc 4- |
| | amino butanol, 2-hydroxy-3-naphthoic acid, Fmoc glycine |
| 64 | Fmoc glycine, Fmoc Benzothienylalanine, Fmoc 4-amino |
| | butanol, 2-hydroxy-3-naphthoic acid, Fmoc glycine |
| 65 | Fmoc Alanine, Fmoc Benzothienylalanine, Fmoc 4-amino |
| | butanol, 2-hydroxy-3-naphthoic acid, Fmoc glycine |
| 66 | Fmoc t-butylglycine, Fmoc Benzothienylalanine, Fmoc 4- |
| | amino butanol, 2-hydroxy-3-naphthoic acid, Fmoc glycine |
| 67 | Fmoc N-Methylphenylalanine, Fmoc Benzothienylalanine, |
| | Fmoc 4-amino butanol, 2-hydroxy-3-naphthoic acid, Fmoc |
| | glycine |
| 68 | Fmoc N,N-Dimethylphenylalanine, Fmoc Benzo- |
| | thienylalanine, Fmoc 4-amino butanol, 2-hydroxy-3- |
| | naphthoic acid, Fmoc glycine |
| 69 | Fmoc N,N,N-Trimethylphenylalanine, Fmoc Benzo- |
| | thienylalanine, Fmoc 4-amino butanol, 2-hydroxy-3- |
| | naphthoic acid, Fmoc glycine |
| 70 | Fmoc Glutamine, Fmoc Benzothienylalanine, Fmoc 4-amino |
| | butanol, 2-hydroxy-3-naphthoic acid, Fmoc glycine |
| 74 | Fmoc Glycine, Fmoc Tyrosine, Fmoc 4-amino butanol, 2- |
| | hydroxy-3-naphthoic acid, Fmoc glycine |
| 78 | Fmoc (2S, 3S) Beta-methyl phenylalanine, Fmoc 4-amino |
| | butanol, 2-hydroxy-3-naphthoic acid, Fmoc glycine |
| 79 | Fmoc (2S, 3R) Beta-methyl phenylalanine, Fmoc 4-amino |
| | butanol, 2-hydroxy-3-naphthoic acid, Fmoc glycine |
| 80 | Fmoc benzothienylalanine, Fmoc 4-amino pentanol, |
| | Fmoc D-Phenylalanine, Fmoc glycine |
| 81 | Fmoc benzothienylalanine, Fmoc 4-amino pentanol, Fmoc L- |
| | Phenylalanine, Fmoc glycine |
| 82 | Fmoc benzothienylalanine, Fmoc 4-amino pentanol, Fmoc D- |
| | 1-Naphthylalanine, Fmoc glycine |
| 83 | Fmoc benzothienylalanine, Fmoc 4-amino pentanol, Fmoc L- |
| | 1-Naphthylalanine, Fmoc glycine |
| 84 | Fmoc benzothienylalanine, Fmoc 4-amino pentanol, Fmoc D- |
| | 2-Naphthylalanine, Fmoc glycine |
| 85 | Fmoc benzothienylalanine, Fmoc 4-amino pentanol, Fmoc L- |
| | 2-Naphthylalanine, Fmoc glycine |
| 86 | Fmoc benzothienylalanine, Fmoc 4-amino pentanol, N- |
| | trifluoroacetyl-2-amino-3-naphthoic acid, Fmoc glycine |
| 87 | Fmoc benzothienylalanine, Fmoc 4-amino pentanol, N- |
| | trifluoroacetyl-Tyrosine, Fmoc glycine |
| 88 | Fmoc tyrosine, Fmoc diamino butane, naphthalene, |
| | 2,3 dicarboxylic acid, glycine |
| 89 | Fmoc tyrosine, Fmoc diamino propane, naphthalene, |
| | 2,3 dicarboxylic acid, glycine |
| 90 | Fmoc tyrosine, Fmoc amino butyric acid, 2-amino-3- |
| | naphthoic acid, glycine |
| 91 | Fmoc tyrosine, Fmoc amino pentanoic acid, 2-amino-3- |
| | naphthoic acid, glycine |
| 92 | Fmoc tyrosine, Fmoc amino ethanol, 2-hydroxy-3-naphthoic |
| | acid, glycine |
| 93 | Fmoc tyrosine, Fmoc amino propanol 2-hydroxy-3-naphthoic |
| | acid, glycine |
| 94 | Fmoc tyrosine, Fmoc amino pentanol, 2-hydroxy-3-naphthoic |
| | acid, glycine |
| 95 | Fmoc tyrosine, Fmoc amino hexanol, 2-hydroxy-3-naphthoic |
| | acid, glycine |
| 96 | Fmoc tyrosine, Fmoc-3-hydroxymethyl aniline, Fmoc 2- |
| | hydroxy-3-naphthoic acid, glycine |
| 99 | Fmoc Tyrosine, Fmoc 4-amino butanol, 2-hydroxy-3- |
| | naphthoic acid, Fmoc glycine, Fmoc amino propanol |
| 100 | Fmoc Tyrosine, Fmoc 4-amino butanol, 2-hydroxy-3- |
| | naphthoic acid, Fmoc glycine |
| 101 | Fmoc Tyrosine, Fmoc 4-amino butanol, 2-hydroxy-3- |
| | naphthoic acid, Fmoc amino ethanol |
| 102 | Fmoc Tyrosine, Fmoc 4-amino butanol, 2-hydroxy-3- |
| | naphthoic acid, Fmoc beta alanine |
| 103 | Fmoc Tyrosine, Fmoc 4-amino butanol, 2-hydroxy-3- |
| | naphthoic acid, Fmoc diaminoethane |
| 104 | Fmoc Tyrosine, Fmoc 4-amino butanol, 2-hydroxy-3- |
| | naphthoic acid, Fmoc piperazine |
| 105 | Fmoc Tyrosine, Fmoc 4-amino butanol, 2-hydroxy-6,9- |
| | dibromo-3-naphthoic acid, Fmoc glycine |
| 106 | Fmoc Tyrosine, Fmoc 4-amino butanol, 2-hydroxy-6- |
| | methoxy-3-naphthoic acid, Fmoc glycine |
| 107 | Fmoc Tyrosine, Fmoc 4-amino butanol, 2-hydroxy-5-bromo- |
| | 3-naphthoic acid, Fmoc glycine |
| 108 | Fmoc Tyrosine, Fmoc 4-amino butanol, 1-hydroxy-2- |
| | naphthoic acid, Fmoc glycine |
| 109 | Fmoc Tyrosine, Fmoc 4-amino butanol, 2-hydroxy-6- |
| | naphthoic acid, Fmoc glycine |
| 110 | Fmoc Tyrosine, Fmoc 4-amino butanol, 2-hydroxy- |
| | quinoxaline-3-carboxylic acid, Fmoc glycine |
| 111 | Fmoc Tyrosine, Fmoc 4-amino butanol, benzofuran-2- |
| | hydroxy-3-carboxylic acid, Fmoc glycine |
| 112 | Fmoc Glycine, Fmoc beta-carboline 2-carboxylic acid |
| 113 | Fmoc Tyrosine, Fmoc 4-amino butanol, 1-hydroxy-2-phenyl- |
| | naphthalene-3-carboxylic acid, Fmoc glycine |
| 114 | Fmoc Tyrosine, Fmoc 4-amino butanol, 3-hydroxy-pyridine- |
| | 2-carboxylic acid, Fmoc glycine |
| 115 | Fmoc Tyrosine, Fmoc 4-amino butanol, 2-hydroxy-5-iodo- |
| | benzoic acid, Fmoc glycine |
| 116 | Fmoc Tyrosine, Fmoc 4-amino butanol, salicylic acid, Fmoc |
| | glycine |
| 117 | Fmoc Tyrosine, Fmoc 4-amino butanol, 2-hydroxy-1- |
| | naphthoic acid, Fmoc glycine, Fmoc-amino-3-propanol |
| 118 | Fmoc Tyrosine, Fmoc 4-amino butanol, 2-hydroxy-7- |
| | methoxy-2-naphthoic acid, Fmoc glycine, Fmoc-amino- |
| | 3-propanol |
| 119 | 4-Amino benzyl alcohol, 2-Hydroxy-3-naphthoic acid, Fmoc |
| | Glycine |
| 121 | 3-Amino benzyl alcohol, 2-Hydroxy-3-naphthoic acid, Fmoc |
| | Glycine |
| 122 | Fmoc Tyrosine, Fmoc 4-amino butanol, 2-hydroxy-3- |
| | naphthoic acid, Fmoc D-Alanine |
| 123 | Fmoc Tyrosine, Fmoc 4-amino butanol, 2-trifluoroacetyl |
| | amino--3-naphthoic acid, Fmoc glycine |
| 125 | Fmoc p-cyano phenylalanine, Fmoc 4-amino butanol, 2- |
| | hydroxy-1-naphthoic acid, Fmoc glycine |
| 126 | Fmoc Tyrosine, 2-amino benzylalcohol, 2-hydroxy-1- |
| | naphthoic acid, Fmoc glycine |
| 136 | Fmoc Tyrosine, Fmoc 4-amino butanol, 2-hydroxy, |
| | 4,5,6 trichlorobenzoic acid, Fmoc glycine, Fmoc- |
| | amino-3-propanol |
| 143 | Fmoc Tyrosine, 3-amino benzylalcohol, 2-hydroxy-1- |
| | naphthoic acid, Fmoc glycine |

Suitable amino acids for the coupling reaction are listed in Table 2 along with the symbol for each amino acid.

TABLE 2

| AMINO ACID | SYMBOL |
|---|---|
| Alanine | Ala or A |
| Arginine | Arg or R |
| Asparagines | Asn or N |
| Aspartic acid | Asp or D |
| Cysteine | Cys or C |
| Glutamine | Gln or Q |
| Glutamic acid | Glu or E |
| Glycine | Gly or G |
| Histidine | His or H |
| Isoleucine | Ile or I |
| Leucine | Leu or L |
| Lysine | Lys or K |
| Methionine | Met or M |
| Phenylalanine | Phe or F |
| Proline | Pro or P |
| Serine | Ser or S |
| Threonine | Thr or T |
| Tryptophan | Trp or W |
| Tyrosine | Tyr or Y |
| Valine | Val or V |

Examples II, III, IV, V, VII, VIII, IX, X, XI, XII, XIII, XIV, XV, XVI, XVII, XVIII and XIX were prepared using Schemes H through R. The definitions in these Schemes apply to the aforementioned Examples.

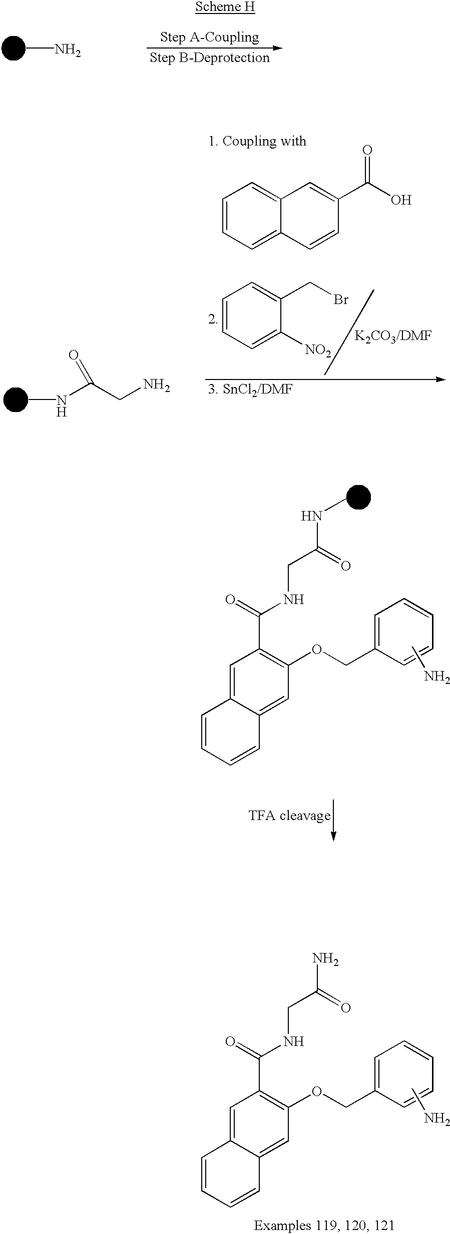

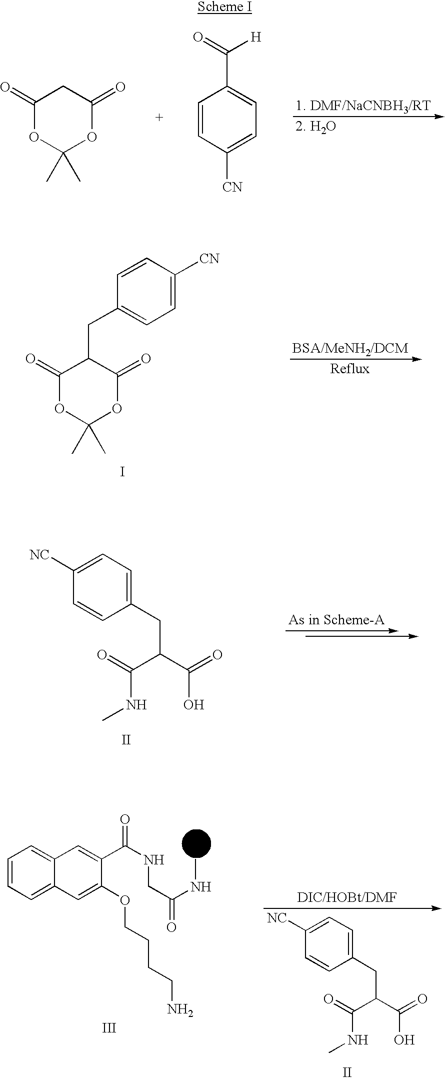

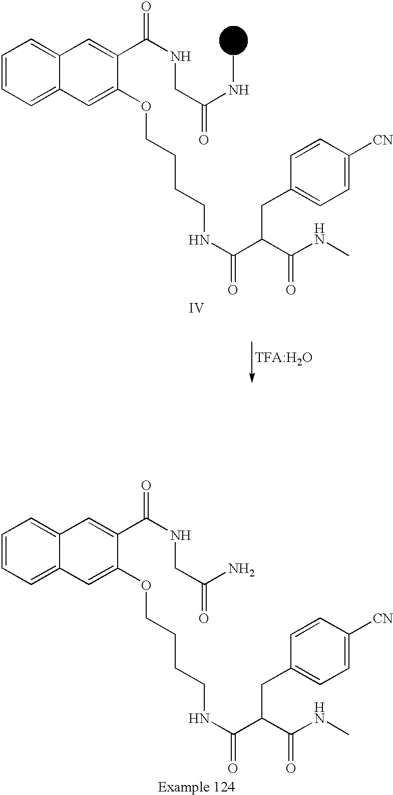

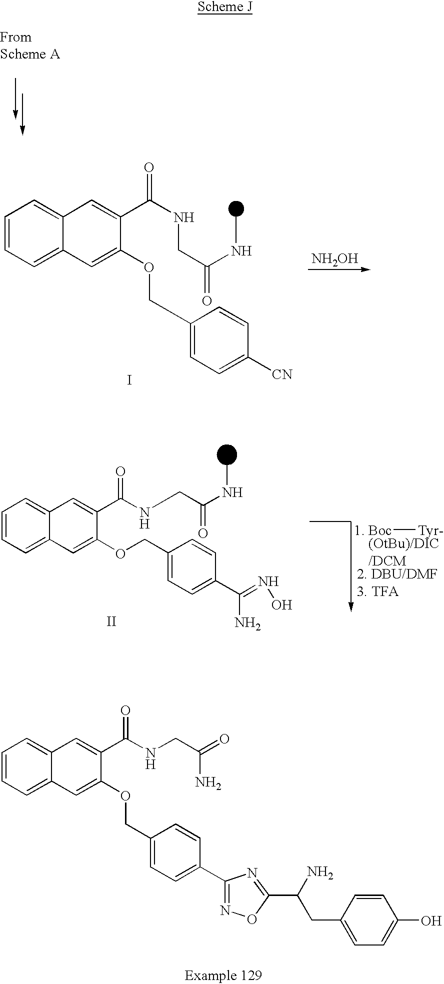

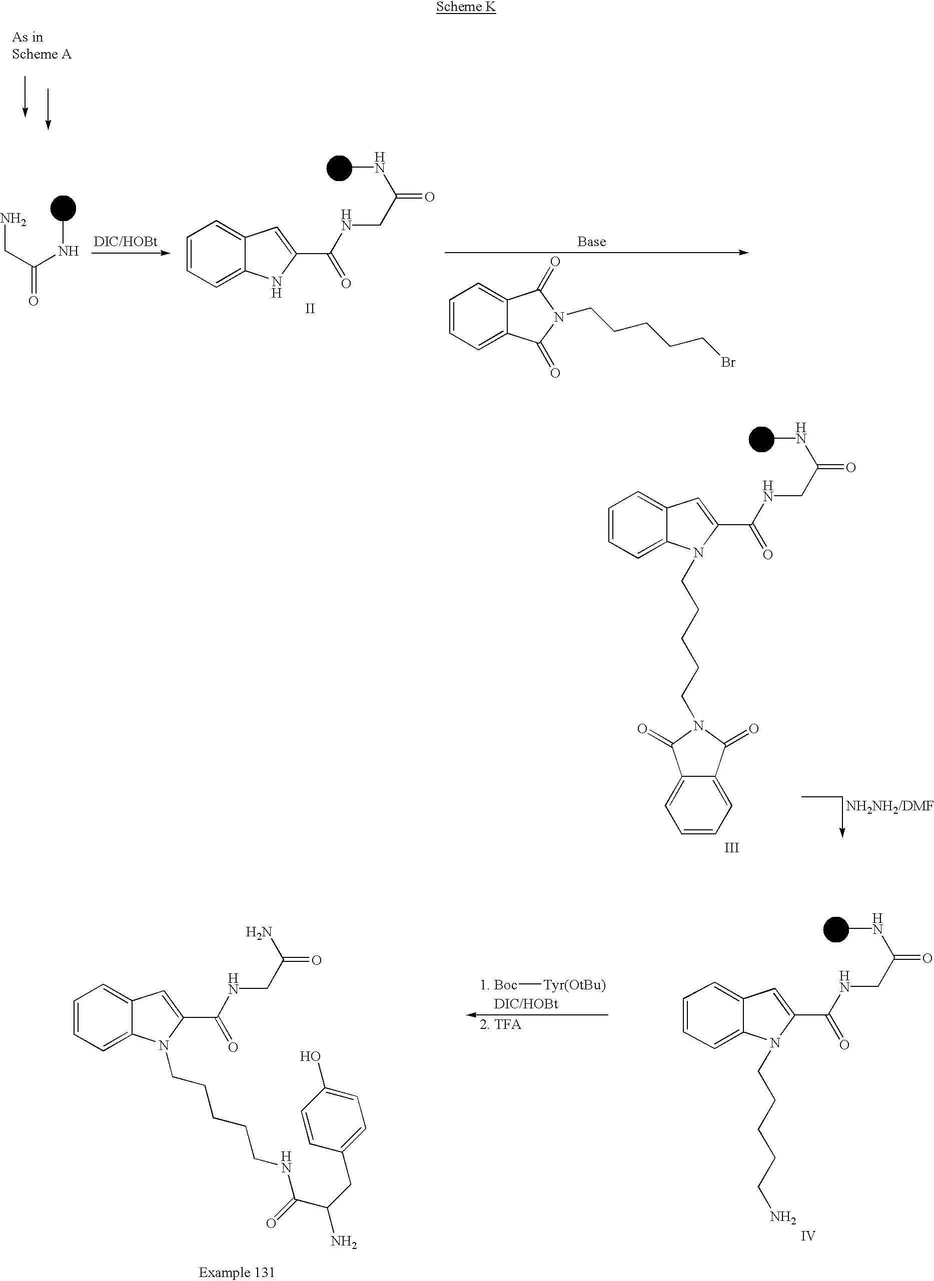

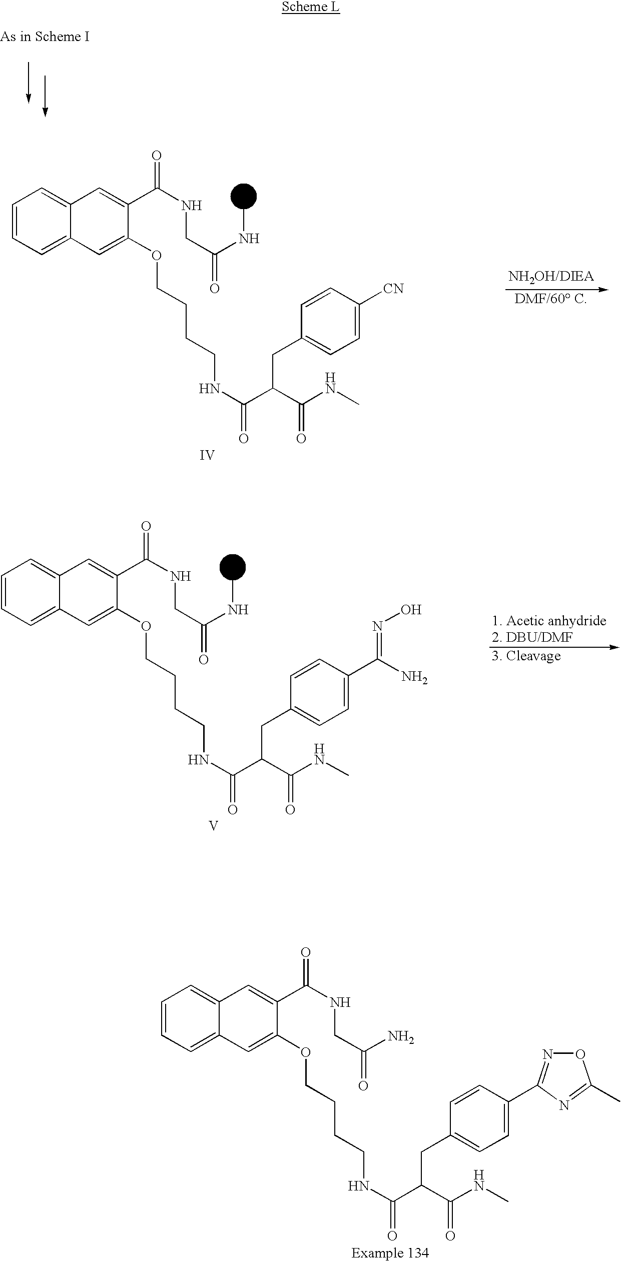

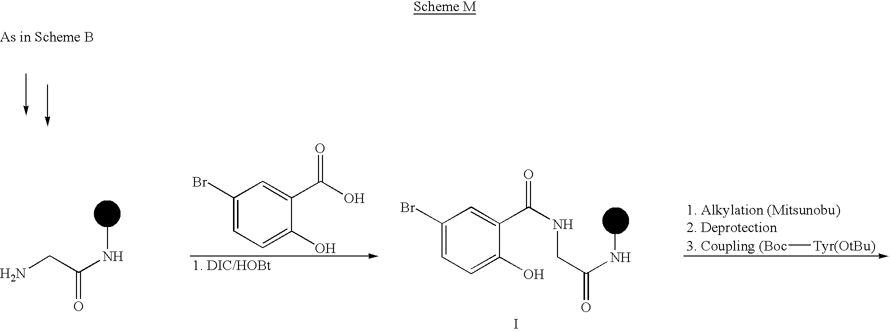

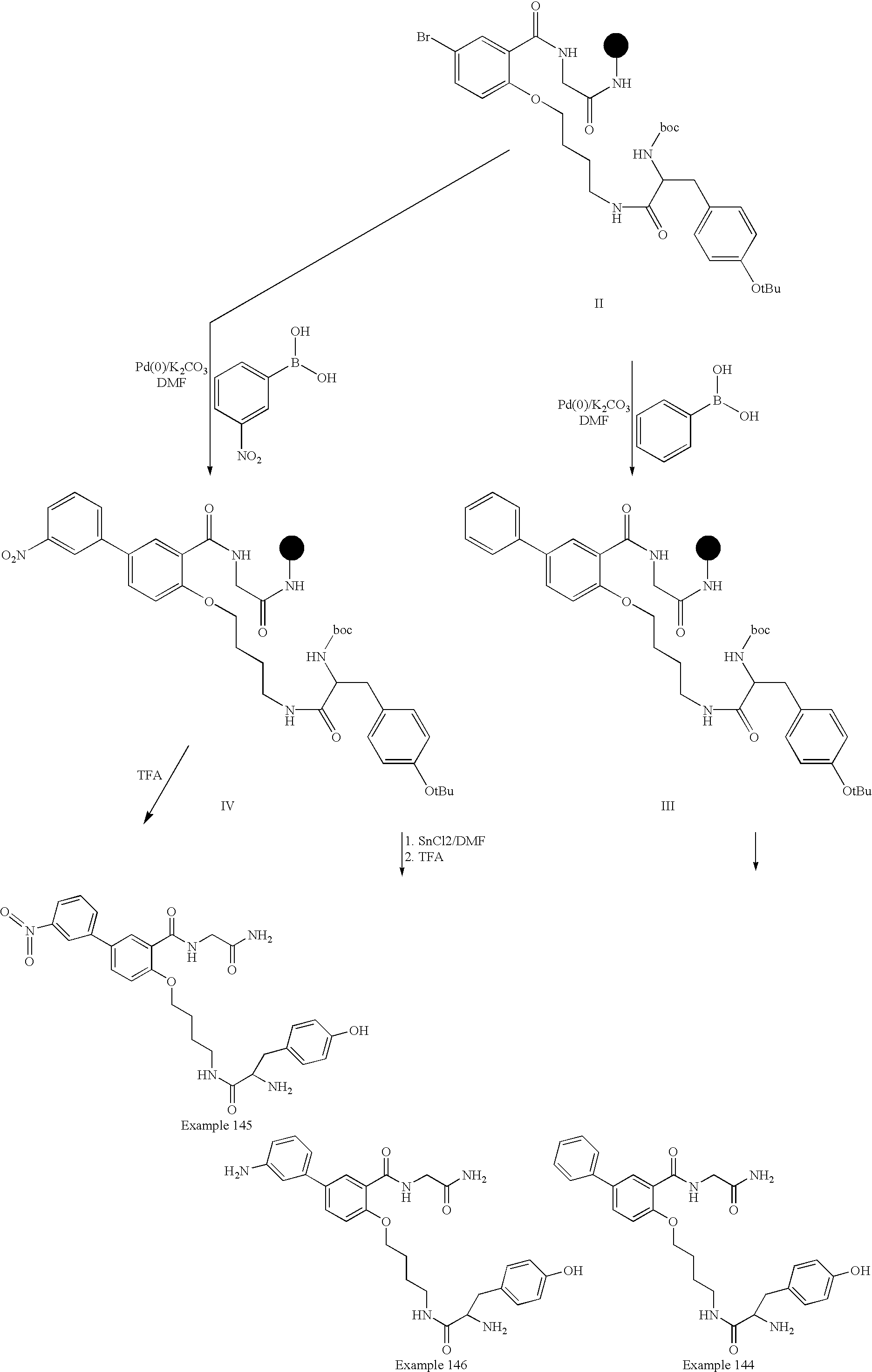

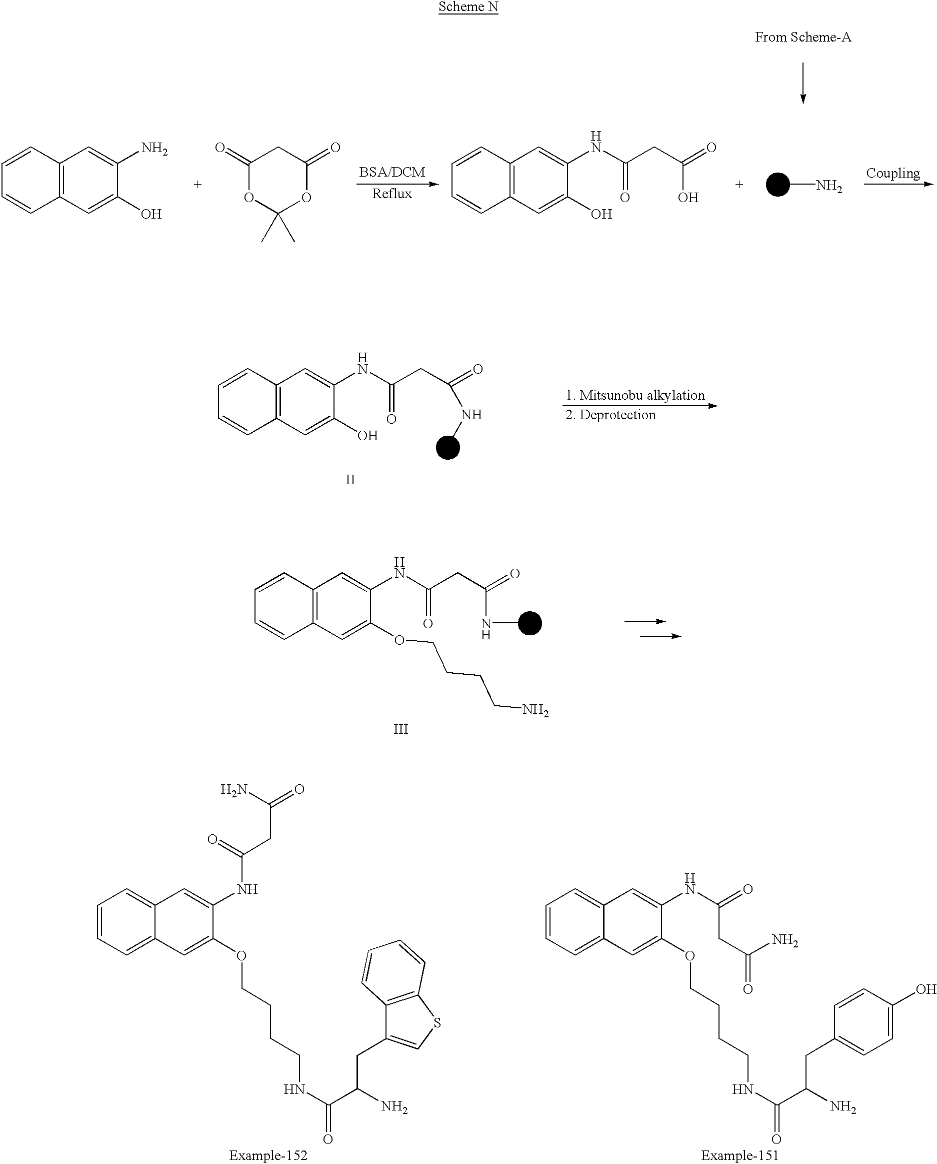

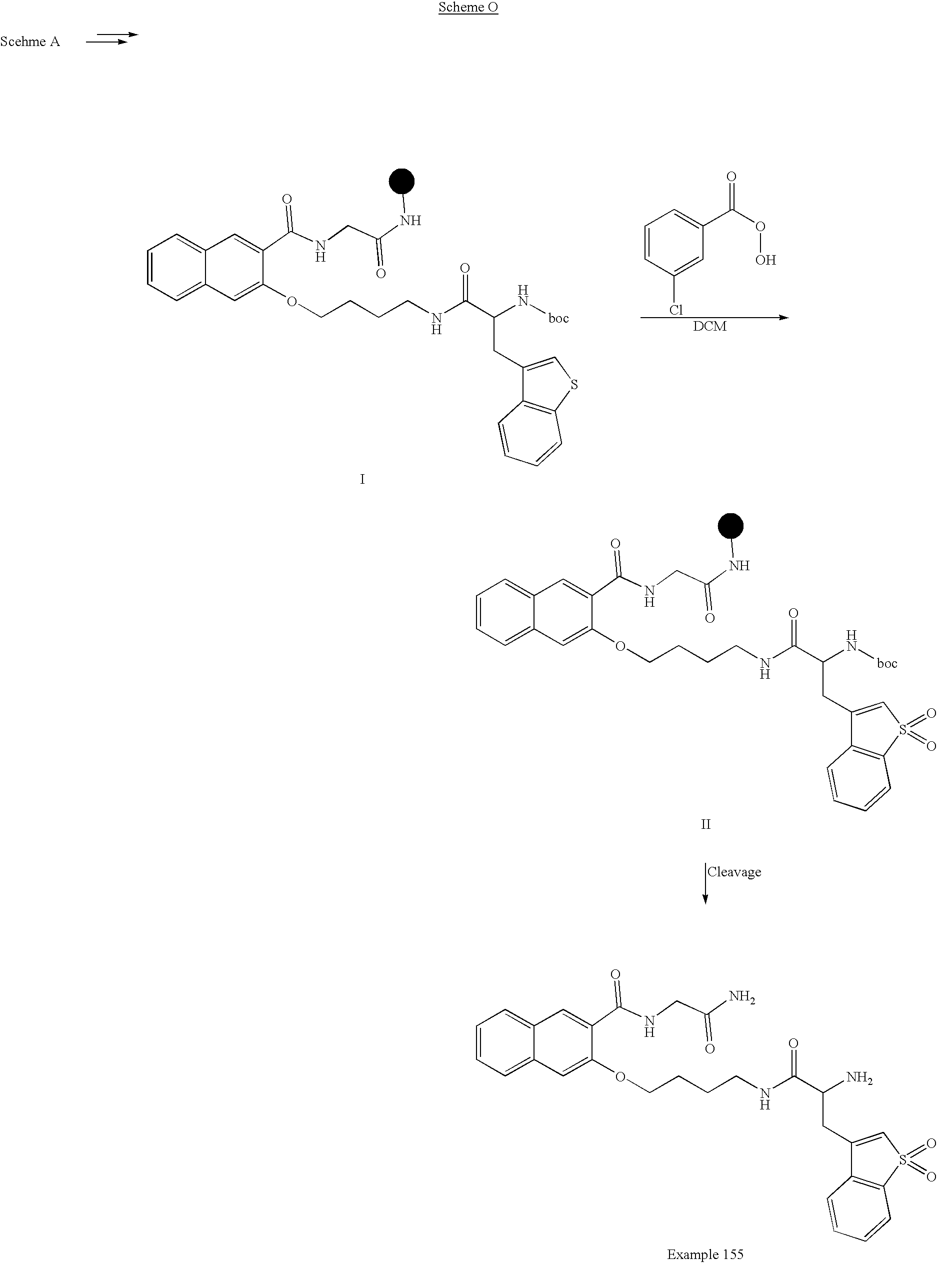

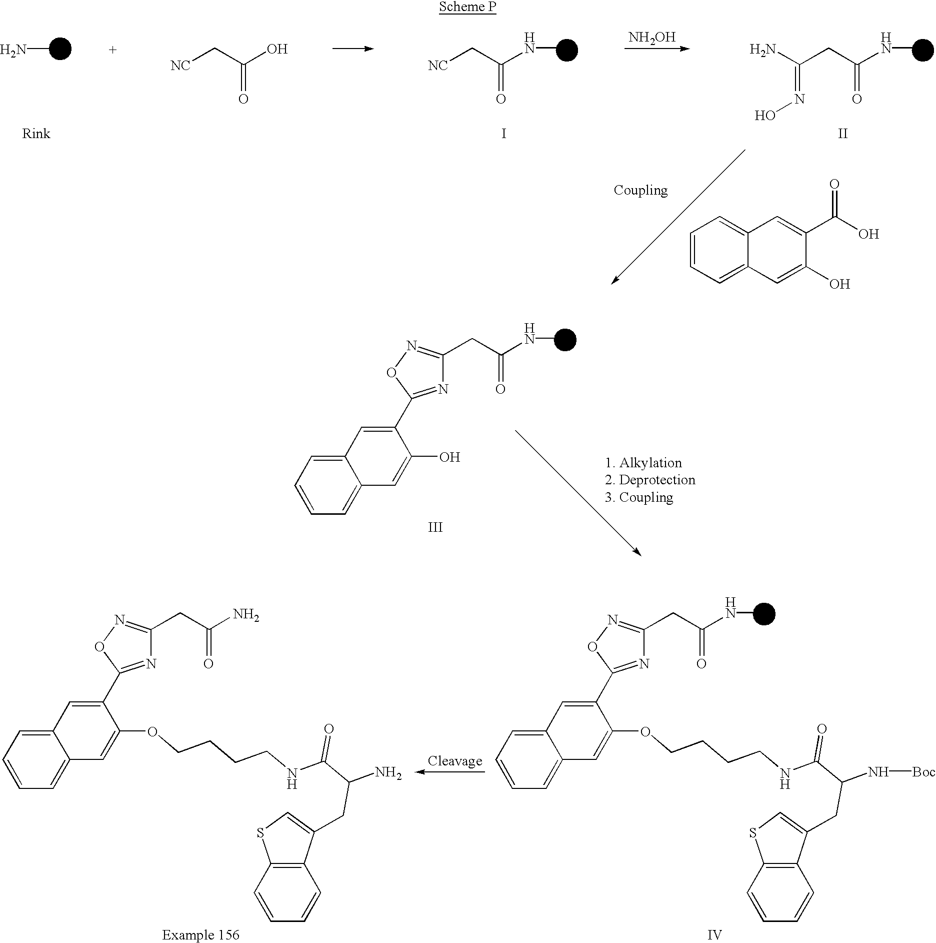

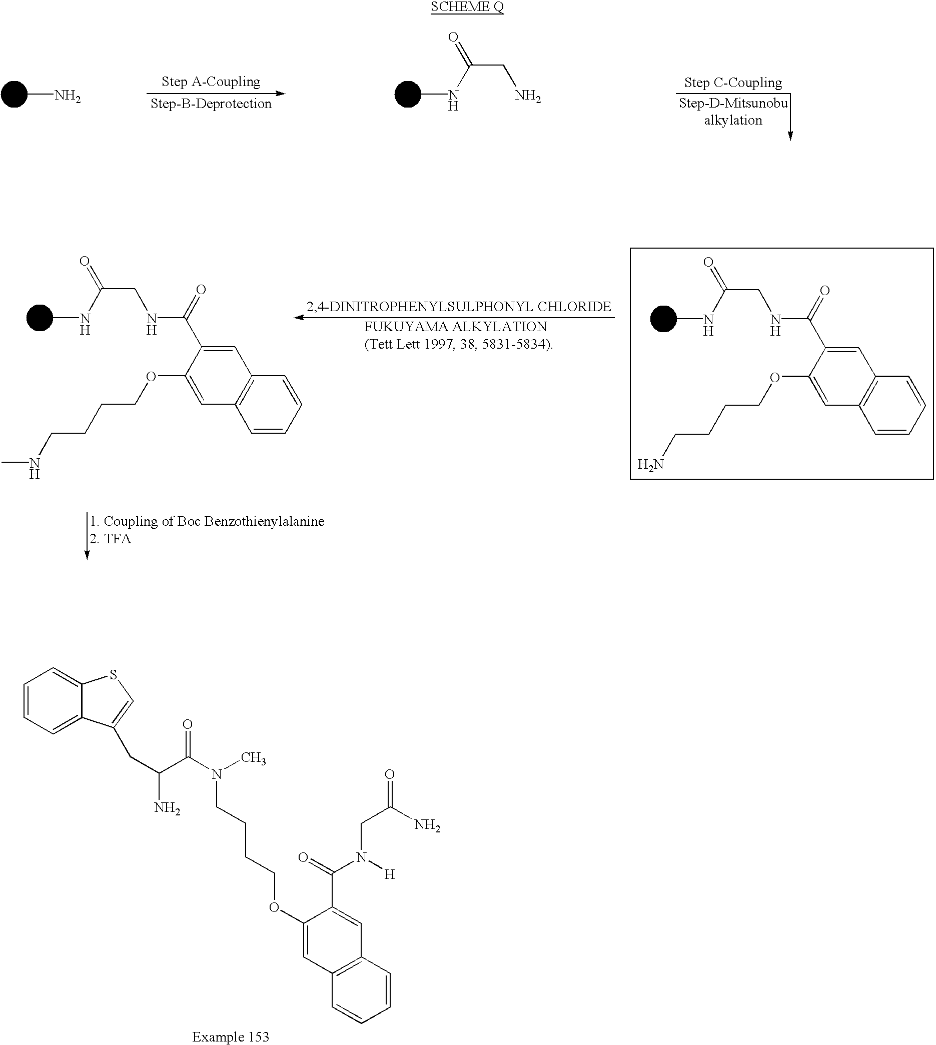

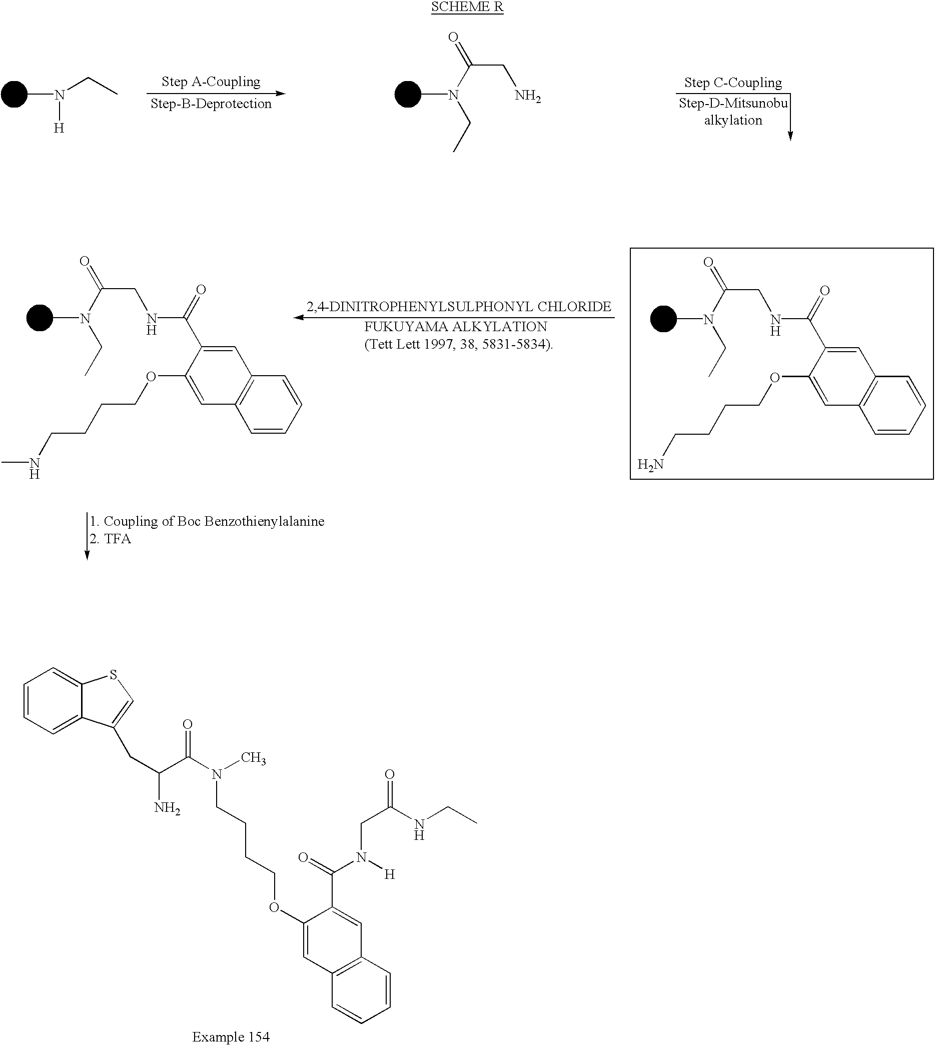

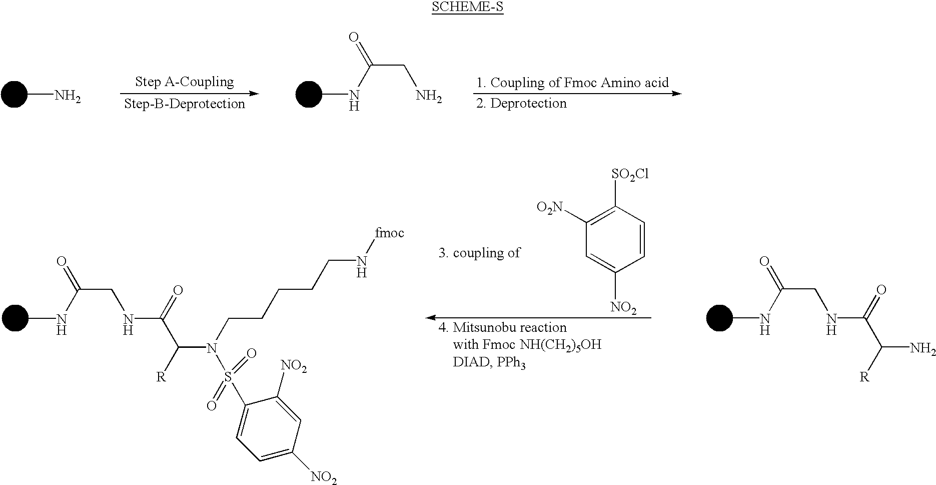

Ref: S. C. Miller, T. S. Scanlan, J.Am.Chem. Soc 1997, 119, 2301-2302.

5. Removal of sulfonyl group (CH₃CH₂CH₂NH₂)

6. Removal of Fmoc (30% Piperidine in DMF)

7. Coupling of Boc Benzothienylalanine

8. Cleavage from Resin

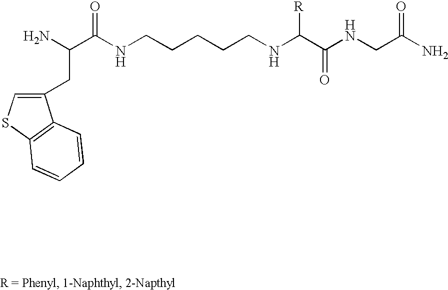

### Examples 80-87, and 123

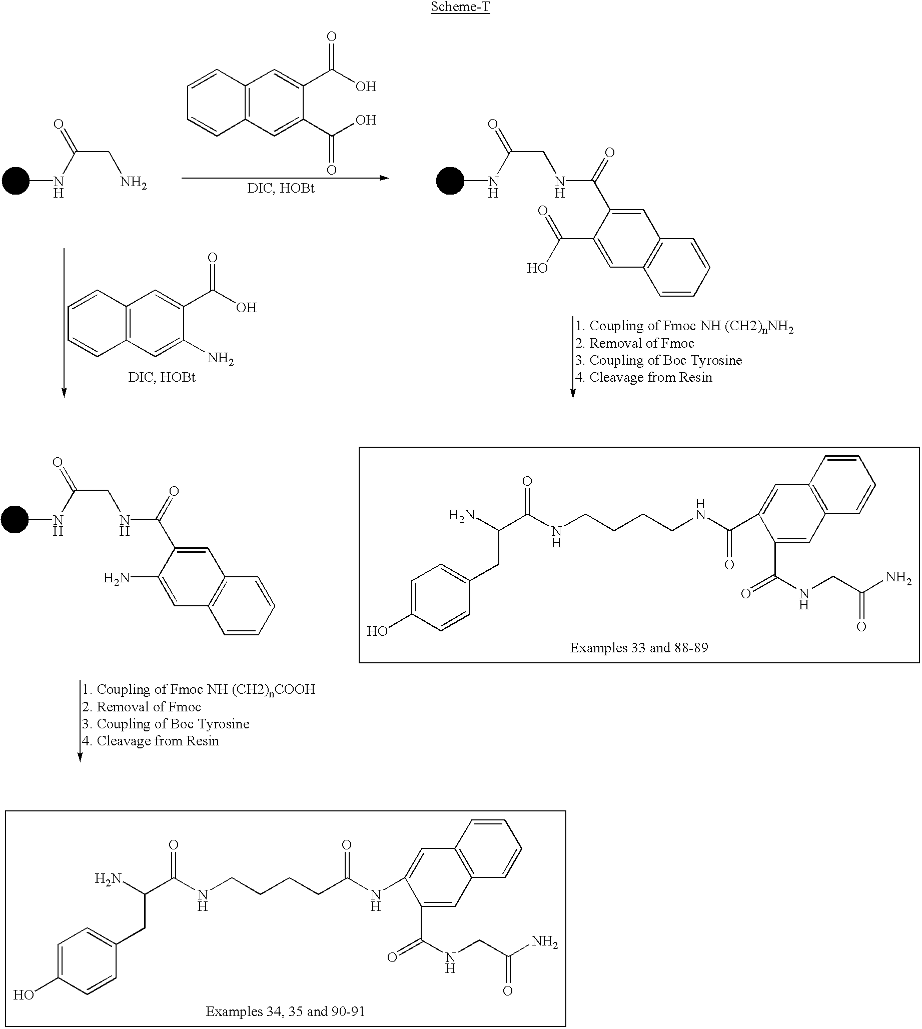

### Example II (Compound 120)

### Example 120: (Scheme H)

### {3-[(2-aminophenyl)methoxy](2-naphthyl)}-N-(carbamoylmethyl)carboxamide

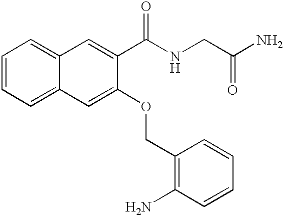

To 66 mg of resin I was added 285 mg K₂CO₃ and 3 mL of DMF followed by 44 mg (0.2 mmol) of 2-nitrobenzyl bromide. The reaction mixture was heated at 50° C. for 6 h. After washing and drying the resin, 40 mg was mixed with 100 mg SnCl₂.2H₂O in DMF containing 165 mg formic acid. The reaction was continued at room temperature for over night. Resin was washed with DMF, MeOH, DCM and dried for 3 h in vacuum. The dried resin was cleaved with TFA:H₂O (95:5) for 1 h at room temperature. Cleavage extracts were evaporated to dryness and passed through C18-Sep-Pak cartridge and then lyophilized from acetonitrile-water to give the compound above. Analysis by HPLC-MS gave mass (M+H:350; cal. 350.1).

### Example III (Compound 121)

### Example 121: (Scheme H)

### {3-[3-aminophenyl)methoxy](2-naphthyl)}-N-(carbamoylmethyl)carboxamide

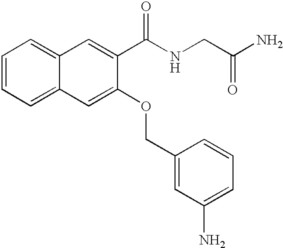

The Procedure as set forth in Example II was employed in this Example III except that 2-nitrobenzyl bromide is replaced with 3-nitrobenzyl bromide to prepare the compound, Example 121.

### Example IV (Compound 119)

### Example 119: (scheme H)

### {3-[(aminophenyl)methoxy](2-naphhtyl)}-N-(carbamoylmethyl)carboxamide

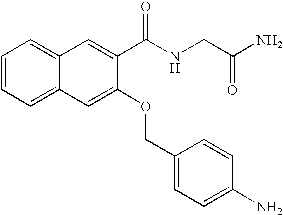

The Procedure as set forth in Example II was employed in this Example IV except that 2-nitrobenzyl bromide is replaced with 4-nitrobenzyl bromide to prepare the compound, Example 119.

### Example V (Compound 124)

### Example 124: (scheme I)

### N-(4-{3-[N-(carbamoylmethyl)carbamoyl](2-naphthyloxy)}butyl)-2RS-[(4-cyanophenyl)methyl]-N′-methylpropane-1,3-diamide

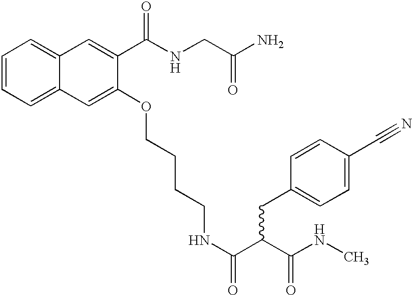

2,2-Dimethyl-5-(p-cyanobenzyl)-1,3-dioxane-4,6-dione (I). (Scheme I) A mixture of Meldrum's acid (28.5 g, 0.198 mol), NaBH₃CN (13.5 g, 0.22 mol) and p-cyano-benzaldehyde (25.8 g, 0.198 mol) in DMF (1000 mL) was stirred at room temperature overnight, then poured into of cold H₂O (7 L). The pH was adjusted to 3.8 with 5% HCl, and the mixture was filtered, rinsed with H₂O and dried to afford 27.7 g (53%) of a white solid: mp 141-143° C.; ¹H NMR (CDCl₃) δ7.60 (d, 2 H, J=8.5 Hz), 7.49 (d, 2 H, J=8.3 Hz), 3.84 (t, 1 H, J=5.1 Hz), 3.53 (d, 2 H, J=5.1 Hz), 1.79 (s, 3 H), 1.67 (s, 3 H); ¹³C NMR (CDCl₃) δ164.67, 142.65, 132.23, 130.69, 118.66, 111.00, 105.33, 47.57, 31.56, 28.27, 26.85; MS (CI/ammonia) m/z 277 (M⁺+NH₄, 100). 3-(4-cyanophenyl)-2-(N-methylcarbamoyl)propionic acid (II): (Scheme I). A solution of methylamine hydrochloride (13.5 g, 200 mmol), anhydrous dichloromethane (200 mL) and BSA (20 g, 100 mmol) was refluxed for 3 h, then cooled to room temperature before compound (I, in Scheme-X1, 5.19 g, 20 mmol) was added. The mixture was refluxed for 3 h, then cooled to room temperature, and carefully poured into cold 1N HCl/EtOAc. The layers were separated, the aqueous extracted again with EtOAc, the organics were combined, washed with brine, dried and stripped to afford 5.72 g (82%) of a white solid: mp 143-145 C (—CO2); ¹H NMR (DMSO-d6) δ12.58 (bs, 1H), 7.97 (t, 1H, J=4.4 Hz), 7.73 (d, 2H, J=8.3 Hz), 7.37 (d, 2H, J=8.3 Hz), 3.51-3.46 (m, 1H), 3.10-3.00 (m, 2H), 2.50 (d, 3H, J=4.6 Hz); ¹³C NMR (DMSO-d6) δ170.56, 167.96, 145.21, 132.03, 129.85, 118.90, 109.05, 53.00, 34.19, 25.67; MS (Cl/ammonia) m/z 206 (M⁺+1, —CO2, 100).

Resin III (200 mg, 0.15 mmol) (Scheme-X-2) was coupled with 3-(4-cyanophenyl)-2-(N-methylcarbamoyl)propionic acid (II) (0.23 mmol.) in presence of HOBt (35 mg; 0.23 mmol) and DIC (36 mg, 0.23 mmol) in DMF (1.5 mL). Coupling continued overnight. Resin was washed with DMF (3×), MeOH (3×), DCM (4×) and dried in vacuum for 4 h before cleavage. The dried resin was cleaved with 95:5 TFA:H₂O for 45 min. Evaporation of cleavage mixture and lyophilization of the product gave 31% yield and >75% purity by HPLC. MS analysis gave M.Wt. 585 (Cal. 585.26).

### Example VI (Compound 125)

### Example 125: Scheme A

### (2S)-2-amino-N-(4-{3-[N-(carbamoylmethyl)carbamoyl](2-naphthyloxy)}butyl)-3-(4-cyanophenyl)propanamide

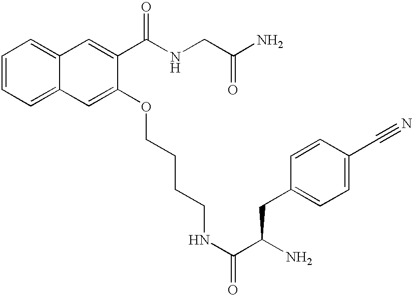

was synthesized according to the procedure used for Example 1 with substitution of Fmoc-Phe(CN)—OH for Fmoc-Tyr(O-tBu)-OH.

### Example VII (Compound 126)

### Example 126: (Scheme A)

### (2S)-2-amino-N[2-({3-[N-(carbamoylmethyl)carbamoyl](2-naphthyloxy)}methyl)phenyl]-3-(4-hydroxyphenyl)propanamide

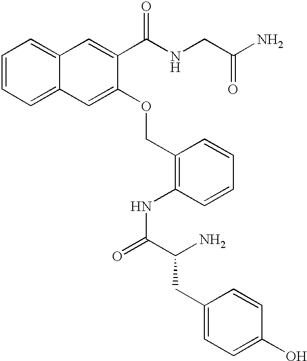

To 150 mg of 3-hydroxy-2-naphtaoyl-Gly-Rink resin was added 97 mg of 2-nitrobenzyl bromide in 1.5 mL DMF and 300 mg K₂CO₃. The resin was heated at 50 C. for 1 h and left at room temperature for 12 h. The resin was washed with DCM/DMF/DMF:H2O ((:1)/DMF/MeOH/DCM and dried in vacuum to give resin I (scheme-X-1). To resin I was added 220 mg SnCl₂.2H₂O in 1.5 mL DMF containing 200 mg acetic acid. The resin was agitated at room temperature for 3 days to give resin II. After washing and drying the resin was coupled with Fmoc-Tyr(OtBu)-OH in THF and in presence of DIC. The finished resin was deprotected, washed and cleaved with TFA:H₂O (95:5). Cleaved product was analyzed with HPLC-MS. The final product gave expected mass (M+H=513; cal. 513).

### Example VIII (Compound 129)

### Example 130 (scheme J)

### {3-[(4-(3-[(1S)-1-amino-2-(4-hydroxyphenyl)ethyl](1,2,4-oxadiazol-5-yl)}(2-naphtyl)}-N-(carbamoylmethyl)carboxamide

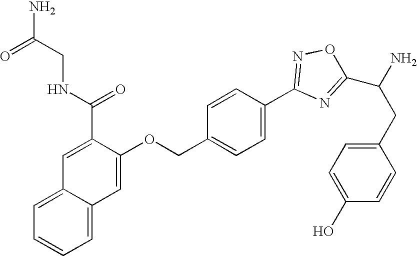

To 150 mg of 3-hydroxy-2-naphtaoyl-Gly-Rink resin was added 109 mg of 4-cyanobenzyl bromide in 1.5 mL DMF and 300 mg K₂CO₃. The resin was heated at 50 C. for 1 h and left at room temperature for 12 h. The resin was washed with DCM/DMF/DMF:H2O ((:1)/DMF/MeOH/DCM and dried in vacuum to give resin I (scheme J). To the dried resin was added a mixture of hydroxylamine hydrochloride (350 mg) in presence of 1.5 mL of 1:1 triethylamine:pyridine in DMF. The resin was agitated at room temperature for 3 days. The finished resin was washed and dried to give resin II (Scheme J). To resin II was added a mixture of Boc-Tyr(OtBu) (169 mg; 0.5 mmol), DIC (35 mg; 0.25 mmol) in 3 mL DCM. The resin reacted for 12 h at room temperature. The resin was washed and re-coupled with Boc-Tyr(OtBu) again under the same conditions. The finished resin was washed and suspended in 3 mL of DMF containing 1 mL of DBU and reacted for 7 h at room temperature. The resin was washed and cleaved as usual to give the final product which was identified by HPLC-MS. The mass of the final compound was (M+H=538.4; cal. 538).

### Example IX (Compound 130)

### Example 129: (Scheme J):

### {3-[(3-(3-[(1S)-1-amino-2-(4-hydroxyphenyl)ethyl](1,2,4-oxadiazol-5-yl)}(2-naphtyl)}-N-(carbamoylmethyl)carboxamide

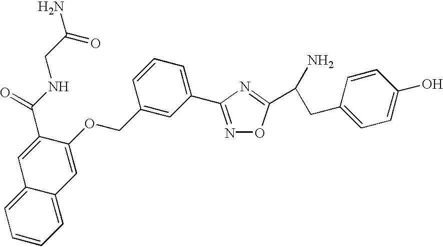

was synthesized according to the procedure used for Example VIII (Compound 129) wherein 3-cyanobenzyl bromide was substituted for 4-cyanobenzyl bromide.

### Example X (Compound 131)

### Example 131: (Scheme K)

### (2S)-2-amino-N-(5-{2-[N-(carbamoylmethyl)carbamoyl]indolyl)pentyl)-3-(4-hydroxyphenyl)propanamide

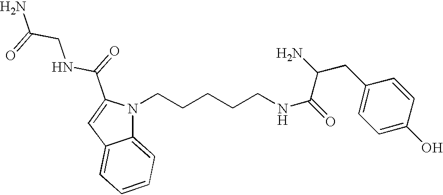

Fmoc-protecting group was removed from 221 mg of Rink resin (loading=0.78 mmol/g) using 50% piperidine in DMF. Unprotected resin was coupled with Fmoc-Gly (310 mg) using DIC/HOBt in DMF as coupling reagents. Fmoc was removed as before and unprotected Gly-Rink resin was coupled with indole-2-carboxylic acid (164 mg) using HOBt (135 mg) and DIC (146 mg) in 2 mL of DMF for 45 min. at room temperature to give resin II (scheme-X-4). To 115 mg of dried resin II was added 140 mg of 2-tert-Butylamino-2-diethylamino-1,3-dimethylperhydro-1,3,2-diazaphosphorine base in 2 mL dimethylacetamide (DMA) and 104 mg of N-(4-bromopentyl)phthalimide . The resin suspension was heated at 60 C. for 8 h. The finished resin III was washed with DMF and treated with 4 mL of DMF containing 30% hydrazine monohydrate for 7 h at room temperature. Unprotected resin was coupled with Boc-Tyr(O-tBu) as usual and the final compound was cleaved by TFA:H₂O (95:5) and analyzed by LC-MS. The product gave the expected mass (M+H=466.6; cal. 466.2)

### Example XI (Compound 134)

### Example 134: (Scheme L)

### N-(4-{3-[N-(carbamoylmethyl)carbamoyl](2-naphthyloxy)}butyl)-N′-methyl-2-{[4-(5-methyl(1,2,4-oxadiazol-3-yl))phenyl]methyl}propane-1,3-diamide

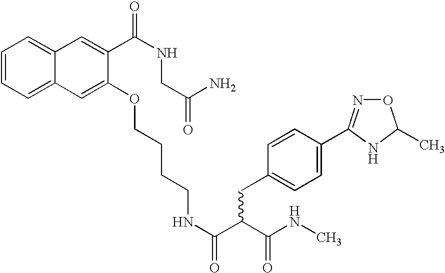

About 70 mg of resin IV (scheme-X-5) synthesized as outlined in Example V (Compound 124) was converted to amidoxime (V) using same procedure described in Example VIII (Compound 129). Resin was washed, dried and reacted with 500 mg of acetic anhydride in DCM for 12 h at room temperature. The resin was washed and reacted with 30% DBU in DMF for another 12 h. The resin was washed, dried and cleaved by TFA:H₂O (95:5) and the product analyzed by LC-MS. The title compound was prepared at 45% purity and gave a mass of 587.2 (M+H); calc. 587.

### Example XII (Compound 135)

### Example 135: (Scheme L)

### (2S)-2-amino-N-(4-{3-[N-(carbamoylmethyl)carbamoyl](2-naphtyloxy)}butyl)-3-[4-(5-methyl(1,2,4-oxadizol-3-yl))phenyl]propanamide

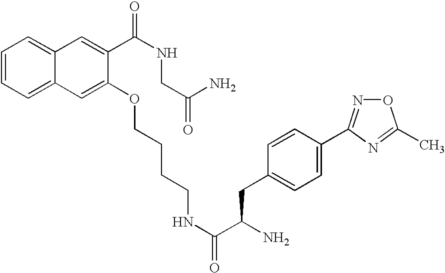

Prepared as outlined in Example XI (Compound 134) except that 3-(4-cyanophenyl)-2-(N-methylcarbamoyl)propionic acid was replaced with Fmoc-Phe(p-CN) acid.

### Example XIII (Compound 136)

### Example 136: (Scheme A):

### (2S)-2-amino-N-(4-{2-[N-(carbamoylmethyl)carbamoyl]-3,4,6-trichlorophenoxy)butyl)-3-(4-hydroxyphenyl)propanamide

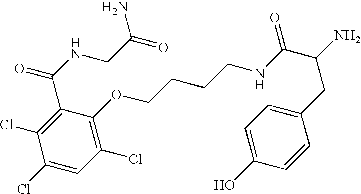

Gly-Rink resin (500 mg; 0.78 mmol/g) prepared as outlined under Example 1 (Scheme-A-1) was coupled with 3,5,6-trichlorosalicylic acid using DIC (150 mg) and HOBt (152 mg) in DMF as a coupling reagents. The resin was processed as described in Example I. The title compound was characterized by LC-MS and purified by HPLC. The purified compound gave expected mass (M+H) of 532.5 (cal. 532.8).

### Example XIV (Compound 144)

### Example 144: (Scheme IV)

### (2S)-2-amino-N-(4-{2-[N-(carbamoylmethyl)carbamoyl]-4-phenylphenoxy}butyl)-3-(4-hydroxyphenyl)propanamide

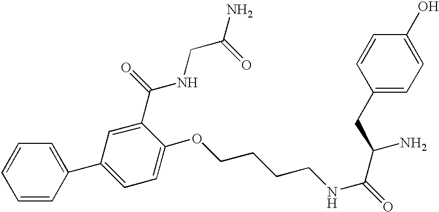

Resin I (scheme M) was prepared as outlined under Example I with substitution of 5-bromosalicylic acid for 3-hydroxy-2-naphthaoic acid. The resin was alkylated with 4-Fmoc-butanolamine under Misunobu's conditions as described before. After Fmoc-deprotection the resin was coupled with Boc-Tyr(OtBu) to give resin II. To 144 mg of resin II was added 70 mg of phenylboronic acid, 240 mg K₂CO₃, 3 mL DMF and 0.3 mL of H₂O. The resin suspension was heated at 75-80 C. for 8 h. The resin was washed with DMF, DMF:H₂O, DMF, MeOH, DCM and dried in vacuum to give resin III. The dried resin was cleaved and the product purified by HPLC. The final compound was identified by LC-MS and give expected mass (M+H) of 505.3; calc. 505.5.

### Example XV (Compound 145)

### Example 145: (Scheme M)

### (2S)-2-amino-N-(4-{2-[N-(carbamoylmethyl)carbamoyl]-4-(3-nitrophenyl)phenoxy}butyl)-3-(4-hydroxyphenyl)propanamide

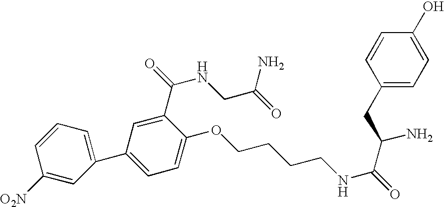

The preparation is the same as set forth in Example XIII (Compound 144) except that phenylboronic acid was replaced with 3-nitrophenylboronic acid.

### Example XVI (Compound 146)

### Example 146: (Scheme M)

### (2S)-2-amino-N-(4-{4-(3-aminophenyl)-2-[N-(carbamoylmethyl)carbamoyl]phenoxy}butyl)-3-(4-hydroxyphenyl)propanamide

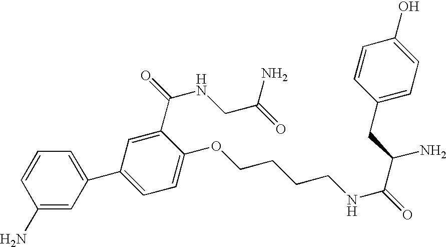

Prepared by reduction of resin IV (scheme M) with SnCl₂.H₂O in DMF as described before in Example 11, Compound 120, and the finished resin was processed as set forth in Example XIII (Compound 144).

### Example XVII (Compound 152)

### Example 152: (Scheme N)

### (2S)-2-amino-3-benzo[b]thiophen-3-yl-N-{4-[3-(2-carbamoylacetylamino)(2-naphthyloxy)]butyl}propanamide

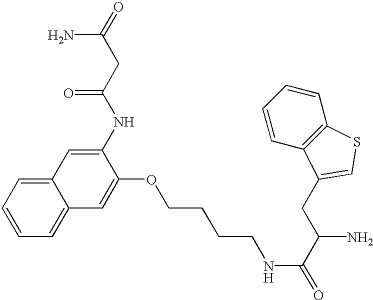

Synthesis of Compound I, Scheme N

In a round bottom flask was mixed 700 mg (4.4 mmol) of 3-amino-2-hydroxynaphthalene, 2 mL of BSA in 30 mL of DCM. The mixture was refluxed for 4 h. To the reaction mixture was added 720 mg (5 mmol) of Meldrum's acid and the mixture refluxed for additional 8 hr. Dichloromethane was rotary evaporated and the residue mixed with ethylacetae (50 mL) and 10 mL of 5% aqueous hydrochloric acid solution. Ethylacetate layer was separated and washed with water, brine and dried over anhydrous Na₂SO₄. Evaporation of organic solvent gave a dark brown solid which was characterized by HPLC and mass spectrometry. Mass spectrometry analysis gave M.Wt. 245.0 (cal. 245.0).

Synthesis of Resin II

Rink resin (1.0 g, 0.6 mmol) was deprotected with 50% Piperidine in DMF (2× for 15 min.) washed with DMF (8×) and coupled with 320 mg (1.3 mmol) of I in presence of 150 mg HOBt (1.0 mmol), 190 mg DIC (1.46 mmol) and 3 mL DMF. Coupling continued at room temperature for 3 h. Ninhydrin tests indicated complete coupling. The finished resin was treated with 5 mL of DMF containing 30% of N-benzyltrimethylammonium hydroxide for 1 h. Resin was washed with DMF (3×), MeOH (3×), 10% glacial acetic acid in DMF (1×), DMF (3×), DCM (4×) and dried in vacuum. A small sample of the finished resin was cleaved with 95:5 TFA:H₂O and analyzed by HPLC and MS indicated the presence of the expected intermediate. Cal. M.Wt. 244.25; Found 244.1.

Synthesis of Resin III

Part of finished resin II (335.6 mg) was swelled and washed with anhydrous THF. To the swelled resin was added 257 mg (1 mmol) triphenylphosphine, 306 mg (1 mmol) of N-Fmoc-n-butanolamine and 3 mL anhydrous THF. Resin suspension was cooled in freezer for 30 min. and 200 μL of Diisopropylazadicarboxylate previously dissolved in 0.5 mL anhydrous THF was added. The coupling reaction was continued for 8 h at room temperature. Resin was washed with DMF (8×), MeOH (4×), DMF (2×) and deprotected with 50% Piperidine in DMF (2×15 min.). Deprotected resin washed with DMF (8×), MeOH (4×), DMF (2×), DCM (4×) and used in the next step. One half of resin III was coupled with N-Boc-Benzthienylalanine (182.5 mg, 0.5 mmol), HOBt (76 mg, 0.5 mmol) and DIC (76 mg, 0.58 mmol) in 1.5 mL DMF. Coupling was completed within 2 h as indicated by ninhydrin test. The finished resin was washed with DMF (4×), MeOH (4×), DCM (6×) and dried in vacuum for 4 h. Dried resin was cleaved with 4 mL of 95:5 TFA:H₂O for 45 min. at room temperature. Evaporation of cleavage mixture and lyophilization of the product gave 50 mg of crude product. The product was purified with HPLC on C₁₈ semipreparative Vydac column. Purified product was >95% pure by HPLC and MS analysis. Calculated M.Wt. 518.4 and found M.Wt. was 518.0.

### Example XVIII (Compound 151)

### Example 151: (Scheme N)

### (2S)-2-amino-N-{4-[3-(2-carbamoylacetylamino)(2-naphthyloxy)]butyl}-3-(4-hydroxyphenyl)propanamide

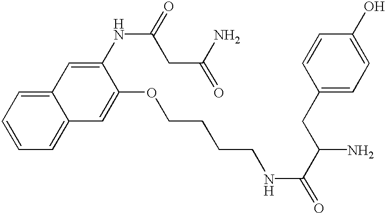

The second half of resin III was similarly treated as outlined under synthesis of Example XVII (Compound 152) except that Boc-Tyr(O-tBu)-OH replaced Boc-Bzt-OH. Final product was purified with HPLC and analyzed with ESI-MS. Purity of final product was >94% and found M.Wt. was 478.0 compared to 478.5 of calculated mass.

### Example XIX (Compound 155)

### Example 155: (Scheme O)

### (2S)-2-amino-3-(1,1-dioxobenzo[b]thiophen-3-yl)-N-(4-{3-[N-(carbamoylmethyl)carbamoyl](2-naphthyloxy)}butyl)propanamide

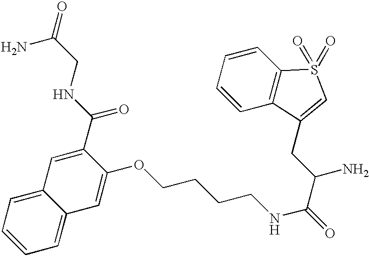

Resin I (Scheme O) was prepared as outlined under Example 1. Aliquot of resin I (160 mg) was suspended in DCM and 99 mg of 3-chlrorperbenzoic acid (mcpb) was added to the resin after cooling in freezer for 10 min. The reaction continued for 8 h at room temperature to give resin II. Resin II was washed, dried and cleaved to give the title compound which was purified by HPLC and identified by MS to give expected mass (M+H) 551.5; cal. 551.2

### Example XX (Compound 156)

### Example 156: (Scheme P)

### (2S)-2-amino-3-benzo[b]thiophen-3-yl-N-(4-{3-[5-(carbamoylmethyl)(1,2,4-oxadiazol-3-yl)](2-naphthyloxy)}butyl)propanamide

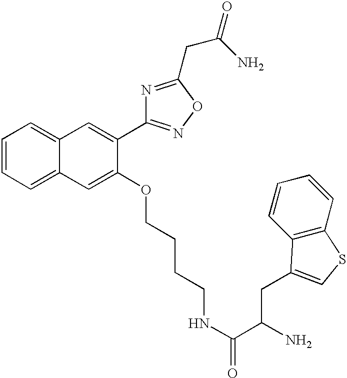

Rink resin (510 mg, loading 0.8 mmol/g) was deprotected and coupled with solution made of cyanoacetic acid (227 mg), HOBt (440 mg) in 3 mL of DCM:DMF (3:7) cooled in freezer for 10 min. before addition of DIC (400 mg) in 1 mL of DCM:DMF (1:1). The reaction continued for about 1 h. Resin was washed and re-coupled for one more time for 4 h to give resin I (scheme-X-9). Resin I was converted to amidoxime as previously described under Example 127 to give resin II. Resin II was coupled to 3-hydroxy-2-naphthaoic acid to give resin III. Resin III was treated as outlined under Example 1 (Scheme A-1) and gave resin IV. Cleavage of resin IV gave the title compound which was purified by HPLC. MS analysis gave expected mass (M+H) 544.6, cal. 544.1.

What is claimed is:

1. A compound, including enantiomers, stereoisomers, rotomers and tautomers of said compound and pharmaceutically acceptable salts, solvates or derivatives thereof, with said compound having the general structure shown in formula I: 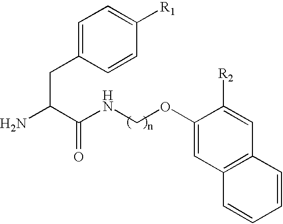
wherein R₁ is OH or hydrogen
R₂ is 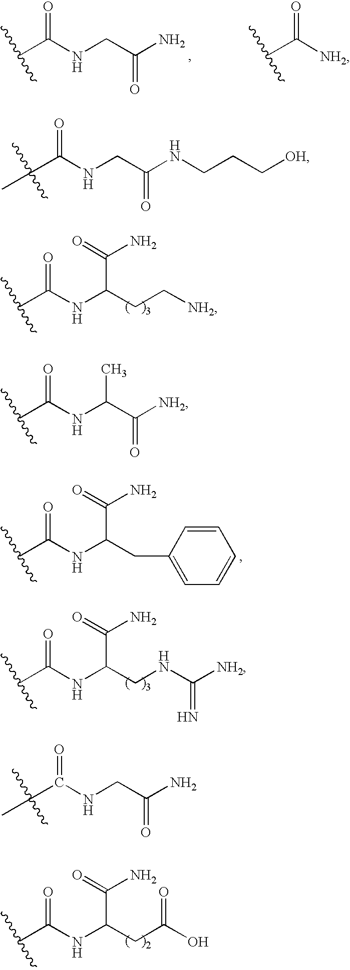
n is an integer having a value of 3 or 4.

2. A compound of claim 1 wherein R₁ is OH and n is 4.

3. A compound of claim 1 wherein R₁ is hydrogen and R₂ is 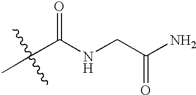
and n is 4.

4. A compound, including enantiomers, stereoisomers, rotomers, and tautomers of said compound and pharmaceutically acceptable salts, solvates or derivatives thereof with said compound having the general structure shown in formula II: 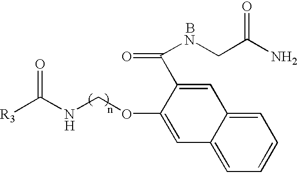
wherein R₃ is 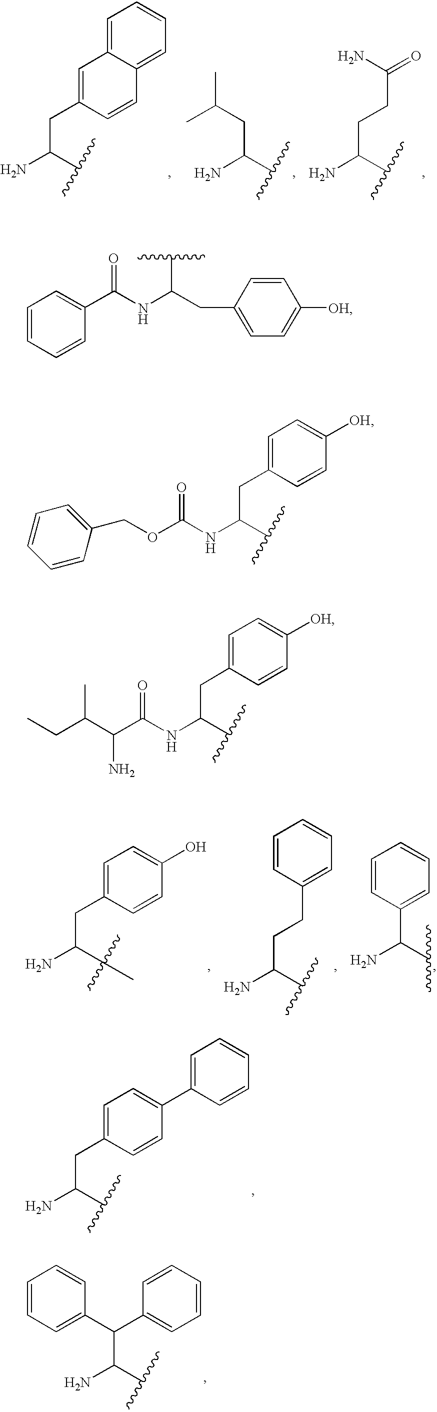 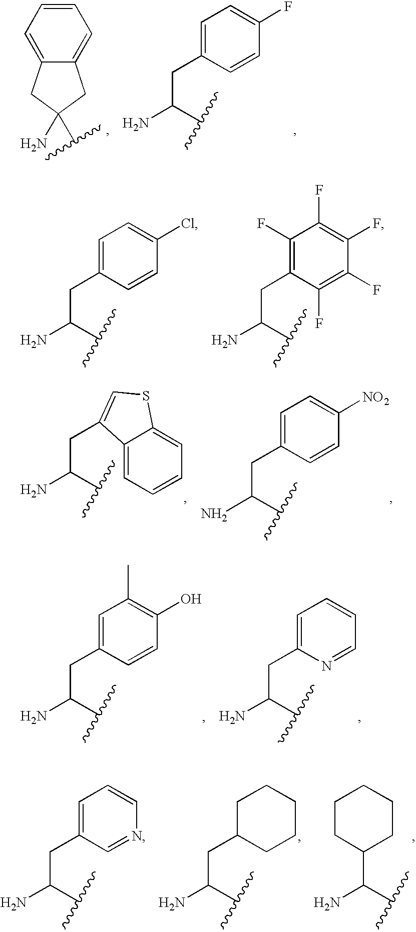
B is —CH₃ or hydrogen
n is an integer having a value of 3 or 4.

5. A compound of claim 4 wherein n is 4 and B is hydrogen.

6. A compound, including enantiomers, stereoisomers, rotomers, and tautomers of said compound and pharmaceutically acceptable salts, solvates, derivates, thereof with said compound having the general structure shown in Formula III: 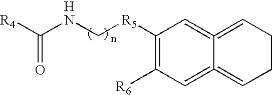
R₄ is 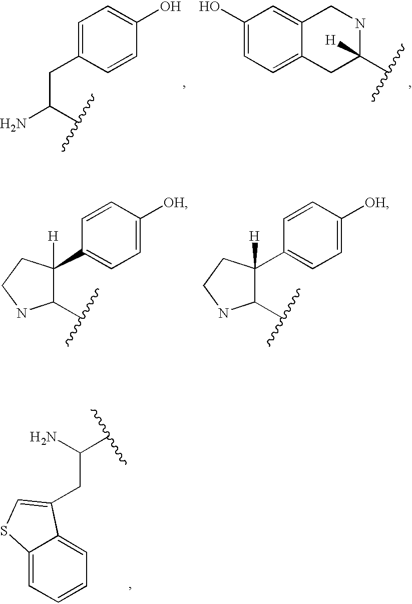
R₅ is either oxygen, nitrogen, 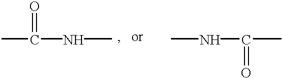
R₆ is 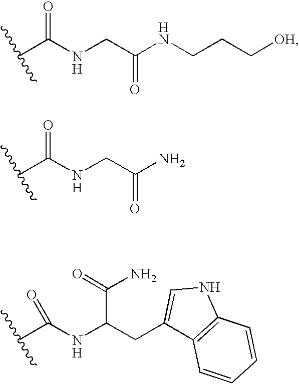
n is an integer having a value of 2 to 6.

7. A compound of claim 6 wherein R₆ is 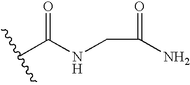
R₅ is oxygen
and n has a value of 4.

8. A compound of claim 7 whereas R₆ is

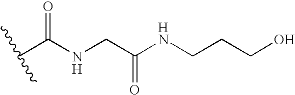

9. A compound, including enantiomers, stereoisomers, rotomers and tautomers of said compound and pharmaceutically acceptable salts, solvates, or derivatives thereof with said compound having the general structure shown in Formula IV: 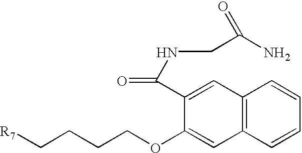
wherein R₇ is

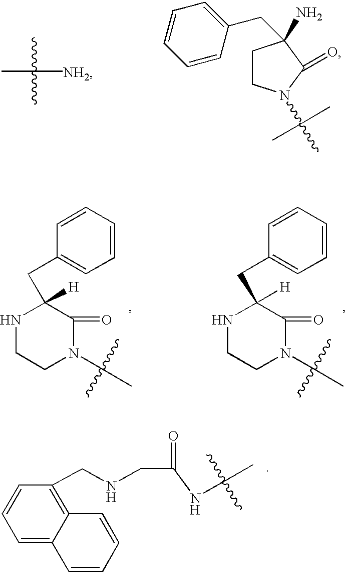

10. A compound, including enantiomers, stereoisomers, rotomers, and tautomers of said compound and pharmaceutically acceptable salts, solvates, or derivatives thereof with said compound having the general structure shown in Formula V: 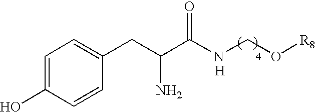
wherein R₈ is

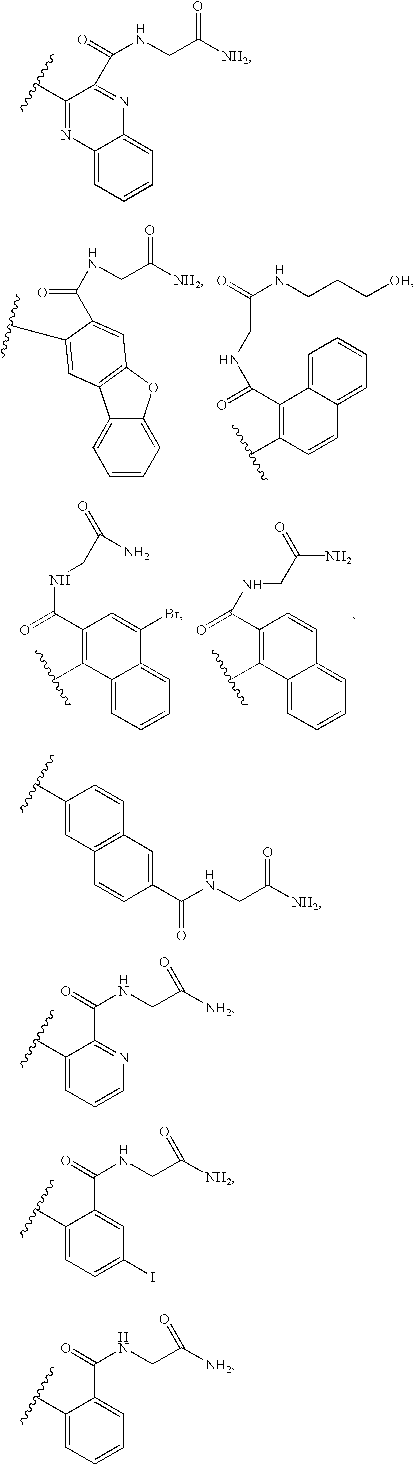

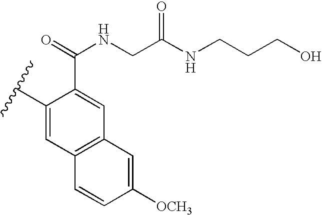

11. A compound, including enantiomers, stereoisomers, rotomers, and tautomers of said compound and pharmaceutically acceptable salts, solvates or derivatives thereof with said compound having the general structure shown in Formula VI: 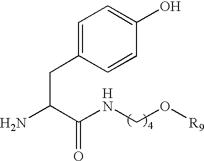
R₉ is

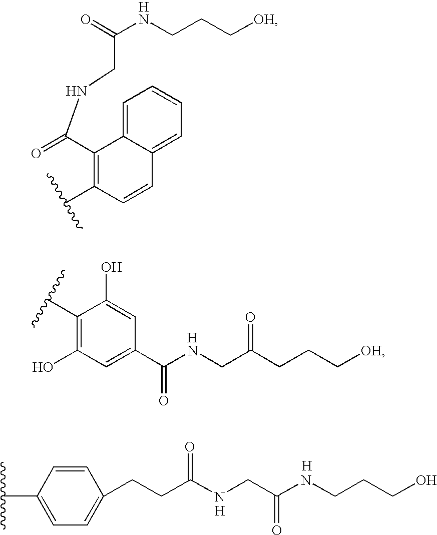

12. A compound, including enantiomers, stereoisomers, rotomers, and tautomers of said compound and pharmaceutically acceptable salts, solvates or derivatives thereof with said compound having the general structure shown in Formula VII: 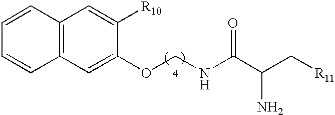
R₁₀ is 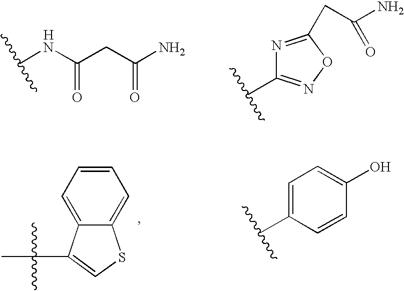
R₁₁ is

13. A compound including enantiomers, stereoisomers, rotomers, and tautomers of said compound and pharmaceutically acceptable salts, solvates or derivatives thereof with said compound having the general structure shown in Formula VIII: 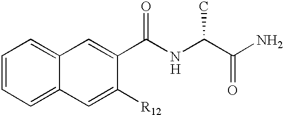
wherein C is hydrogen or methyl, and R₁₂ is

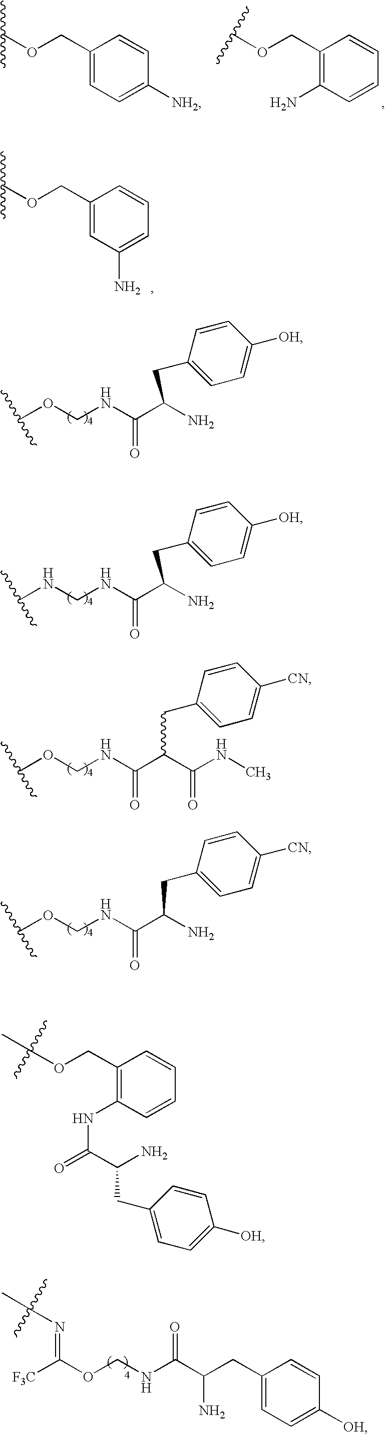

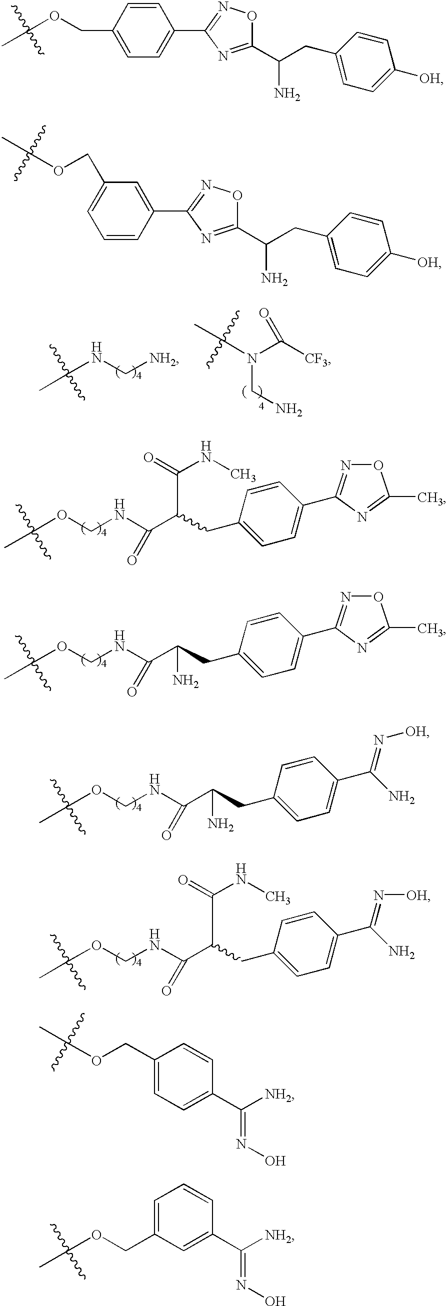

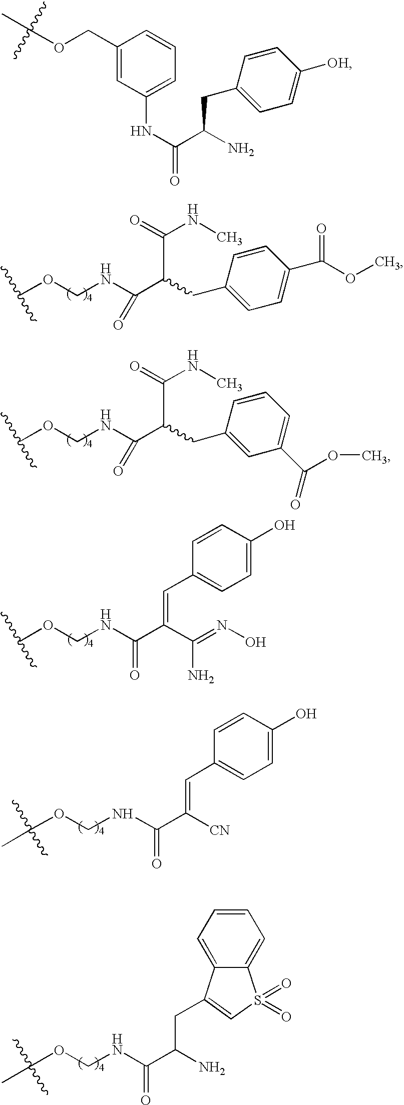

14. A compound, including enantiomers, stereoisomers, rotomers and tautomers of said compound and pharmaceutically acceptable salts, solvates or derivatives thereof, with said compound having the general structure shown in Formula IX: 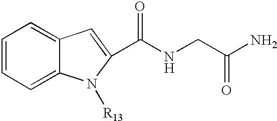
R₁₃ is

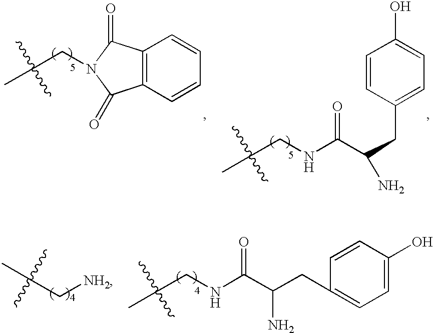

15. A compound, including enantiomers, stereoisomers, rotomers, and tautomers, of said compound and pharmaceutically acceptable salts, solvates, or derivatives thereof, with said compound having the general structure shown in Formula X: 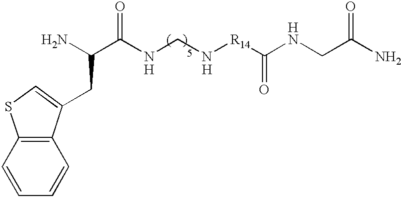
R₁₄ is

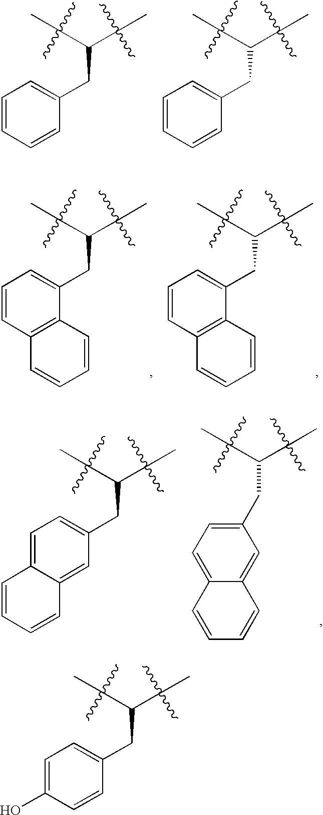

16. A compound, including enantiomers, stereoisomers, rotomers and tautomers of said compound and pharmaceutically acceptable salts, solvates, or derivative, thereof, with said compound having the general structure shown in Formula XI: 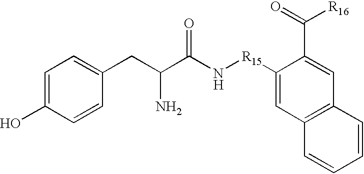
R₁₅ is 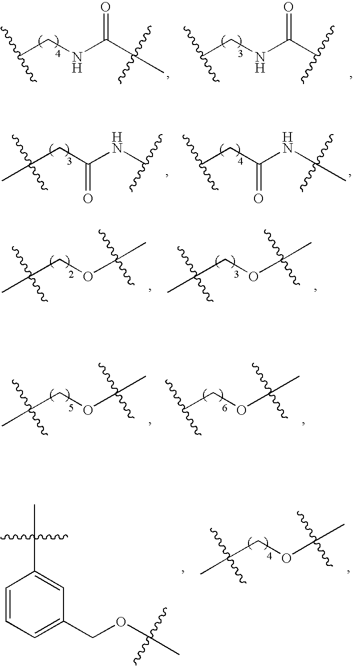
R₁₆ is

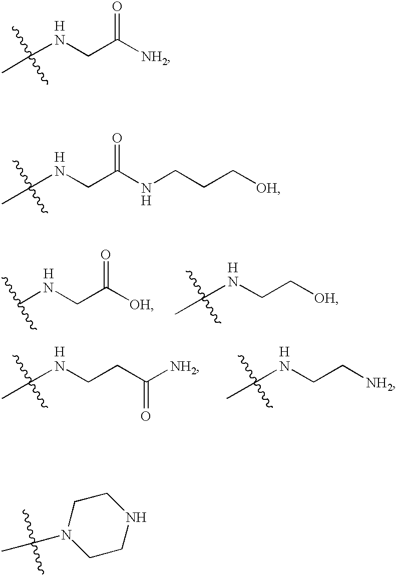

17. The compound of claim 16 wherein R₁₆ is

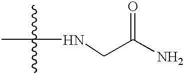

18. The compound of claim 16 wherein R₁₅ is 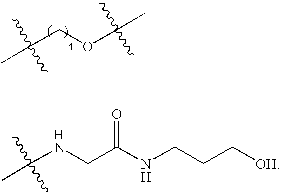
and R₁₆ is

19. A compound, including enantiomers, stereoisomers, rotomers and tautomers of said compound and pharmaceutically acceptable salts, solvates, or derivatives, thereof, with said compound having the general structure shown in Formula XII: 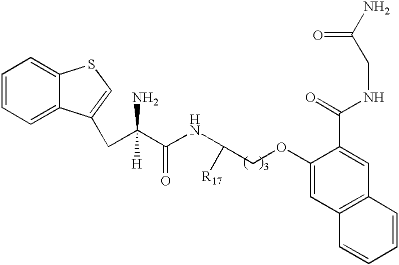
R₁₇ is:

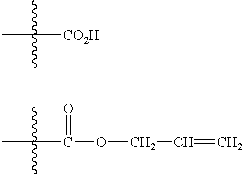

20. A compound, including enantiomers, stereoisomers, rotomers and tautomers of said compound and pharmaceutically acceptable salts solvates, or derivatives, thereof with said compound having the general structure shown in Formula XIII: 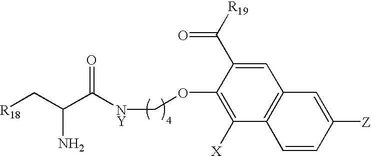
wherein R₁₈ is 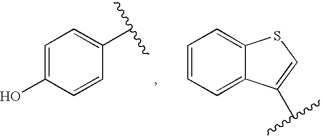
R₁₉ is 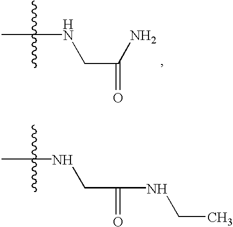
Y is —CH₃ or hydrogen
X is hydrogen or bromine
Z is bromine, —OCH₃, hydrogen.

21. The compound of claim 20 wherein R₁₈ is 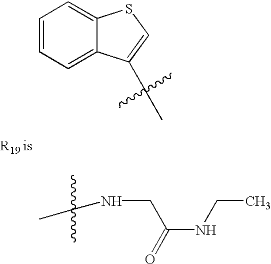
R₁₉ is
Y is —CH₃
X is hydrogen
Z is hydrogen.

22. A compound, including enantiomers, stereoisomers, rotomers and tautomers, of said compound and pharmaceutically acceptable salts, solvates, or derivatives, thereof with said compound having the general structure shown in Formula XIV: 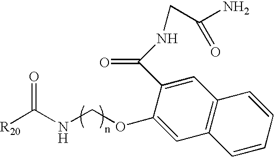
R₂₀ is 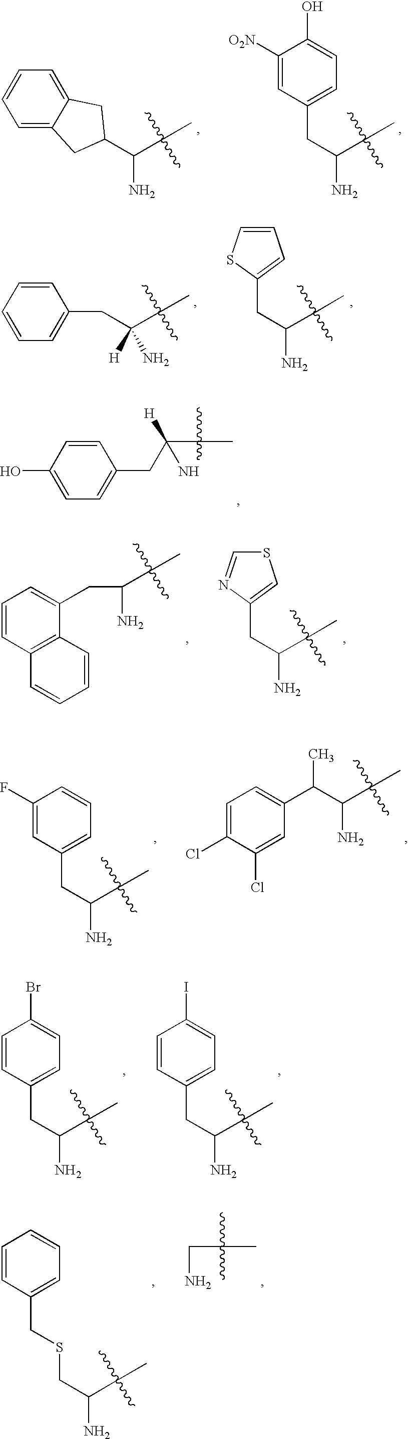 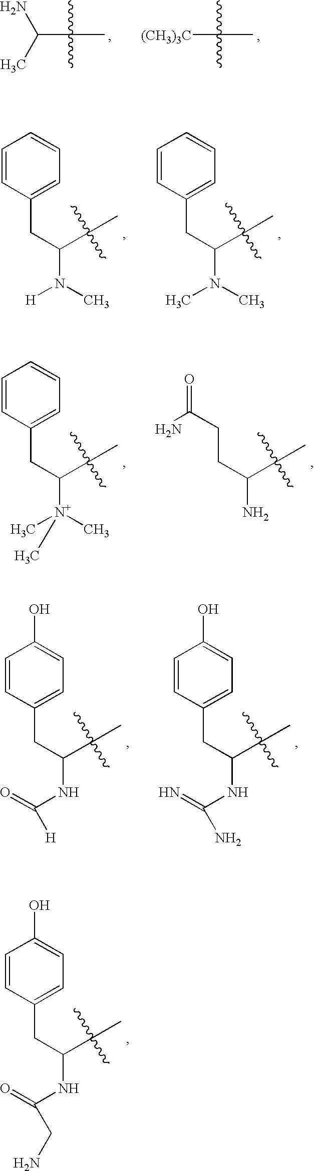
n is an integer having a value of 3 or 4.

23. A compound including enantiomers, stereoisomers, rotomers, and tautomers of said compound and pharmaceutically acceptable salts, solvates, or derivatives thereof with said compound having the general structure shown in Formula XV: 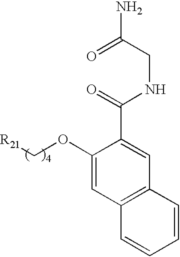
wherein R₂₁ is

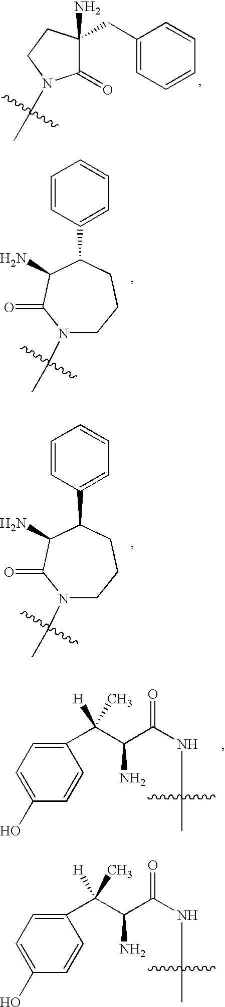

24. A compound including enantiomers, stereoisomers, rotomers, and tautomers of said compound and pharmaceutically acceptable salts, solvates or derivatives thereof with said compound having the general structure shown in Formula XVI: 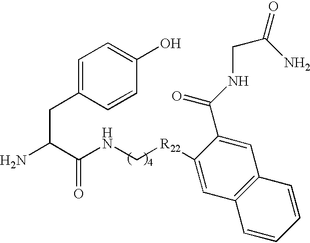
wherein R₂₂ is

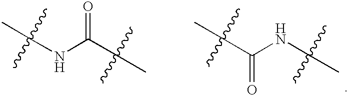

25. A compound including enantiomers, stereoisomers, rotomers and tautomers of said compound and pharmaceutically acceptable salts, solvates, or derivatives, thereof with said compound having the general structure shown in Formula XVII: 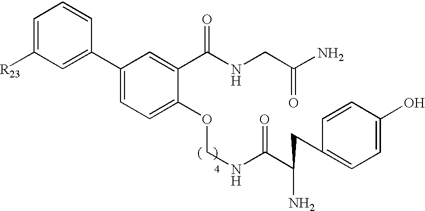
wherein R₂₃ is

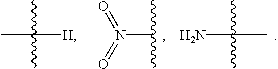

26. A compound including enantiomers, stereoisomers, rotomers, and tautomers of said compound and pharmaceutically acceptable salts, solvates or derivatives thereof with said compound having the general structure shown in Formula XVIII: 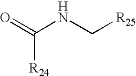
wherein R₂₄ is 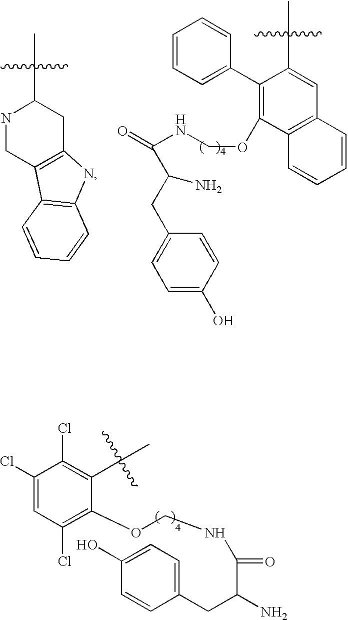
R₂₅ is

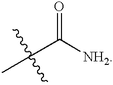

27. A pharmaceutical composition comprising a compound as set forth in claim 1 or a pharmaceutically acceptable salt thereof.

28. A pharmaceutical composition comprising a compound as set forth in claim 4 or a pharmaceutically acceptable salt thereof.

29. A pharmaceutical composition comprising a compound as set forth in claim 6 or a pharmaceutically acceptable salt thereof.

30. A pharmaceutical composition comprising a compound as set forth in claim 9 or a pharmaceutically acceptable salt thereof.

31. A pharmaceutical composition comprising a compound as set forth in claim 10 or a pharmaceutically acceptable salt thereof.

32. A pharmaceutical composition comprising a compound as set forth in claim 11 or a pharmaceutically acceptable salt thereof.

33. A pharmaceutical composition comprising a compound as set forth in claim 12 or a pharmaceutically acceptable salt thereof.

34. A pharmaceutical composition comprising a compound as set forth in claim 13 or a pharmaceutically acceptable salt thereof.

35. A pharmaceutical composition comprising a compound as set forth in claim 14 or a pharmaceutically acceptable salt thereof.

36. A pharmaceutical composition comprising a compound as set forth in claim 15 or a pharmaceutically acceptable salt thereof.

37. A pharmaceutical composition comprising a compound as set forth in claim 16 or a pharmaceutically acceptable salt thereof.

38. A pharmaceutical composition comprising a compound as set forth in claim 19 or a pharmaceutically acceptable salt thereof.

39. A pharmaceutical composition comprising a compound as set forth in claim 20 or a pharmaceutically acceptable salt thereof.

40. A pharmaceutical composition comprising a compound as set forth in claim 22 or a pharmaceutically acceptable salt thereof.

41. A pharmaceutical composition comprising a compound as set forth in claim 23 or a pharmaceutically acceptable salt thereof.

42. A pharmaceutical composition comprising a compound as set forth in claim 24 or a pharmaceutically acceptable salt thereof.

43. A pharmaceutical composition comprising a compound as set forth in claim 25 or a pharmaceutically acceptable salt thereof.

44. A pharmaceutical composition comprising a compound as set forth in claim 26 or a pharmaceutically acceptable salt thereof.

45. A method of treating osteoporosis in humans through the direct inhibition of the Src kinase comprising administering a pharmaceutically effective amount of a compound as set forth in claim 1.

46. A method of treating osteoporosis in humans through the direct inhibition of the Src kinase comprising administering a pharmaceutically effective amount of a compound as set forth in claim 4.

47. A method of treating osteoporosis in humans through the direct inhibition of the Src kinase comprising administering a pharmaceutically effective amount of a compound as set forth in claim 6.

48. A method of treating osteoporosis in humans through the direct inhibition of the Src kinase comprising administering a pharmaceutically effective amount of a compound as set forth in claim 9.

49. A method of treating osteoporosis in humans through the direct inhibition of the Src kinase comprising administering a pharmaceutically effective amount of a compound as set forth in claim 10.

50. A method of treating osteoporosis in humans through the direct inhibition of the Src kinase comprising administering a pharmaceutically effective amount of a compound as set forth in claim 11.

51. A method of treating osteoporosis in humans through the direct inhibition of the Src kinase comprising administering a pharmaceutically effective amount of a compound as set forth in claim 12.

52. A method of treating osteoporosis in humans through the direct inhibition of the Src kinase comprising administering a pharmaceutically effective amount of a compound as set forth in claim 13.

53. A method of treating osteoporosis in humans through the direct inhibition of the Src kinase comprising administering a pharmaceutically effective amount of a compound as set forth in claim 14.

54. A method of treating osteoporosis in humans through the direct inhibition of the Src kinase comprising administering a pharmaceutically effective amount of a compound as set forth in claim 15.

55. A method of treating osteoporosis in humans through the direct inhibition of the Src kinase comprising administering a pharmaceutically effective amount of a compound as set forth in claim 16.

56. A method of treating osteoporosis in humans through the direct inhibition of the Src kinase comprising administering a pharmaceutically effective amount of a compound as set forth in claim 19.

57. A method of treating osteoporosis in humans through the direct inhibition of the Src kinase comprising administering a pharmaceutically effective amount of a compound as set forth in claim 20.

58. A method of treating osteoporosis in humans through the direct inhibition of the Src kinase comprising administering a pharmaceutically effective amount of a compound as set forth in claim 22.

59. A method of treating osteoporosis in humans through the direct inhibition of the Src kinase comprising administering a pharmaceutically effective amount of a compound as set forth in claim 23.

60. A method of treating osteoporosis in humans through the direct inhibition of the Src kinase comprising administering a pharmaceutically effective amount of a compound as set forth in claim 24.

61. A method of treating osteoporosis in humans through the direct inhibition of the Src kinase comprising administering a pharmaceutically effective amount of a compound as set forth in claim 25.

62. A method of treating osteoporosis in humans through the direct inhibition of the Src kinase comprising administering a pharmaceutically effective amount of a compound as set forth in claim 26.
